(19) United States
(12) Patent Application Publication
(10) Pub. No.: US 2024/0376542 A1
(43) Pub. Date: Nov. 14, 2024

(54) LINKED TRANSCRIPT SEQUENCING

(71) Applicant: Singular Genomics Systems, Inc., San Diego, CA (US)

(72) Inventor: Eli N. Glezer, Del Mar, CA (US)

(21) Appl. No.: 18/537,540

(22) Filed: Dec. 12, 2023

Related U.S. Application Data
(63) Continuation of application No. PCT/US2022/035022, filed on Jun. 24, 2022.
(60) Provisional application No. 63/215,371, filed on Jun. 25, 2021.

(51) Int. Cl. C12Q 1/6881; C12Q 1/6844; C12Q 1/6874

(52) U.S. Cl. CPC C12Q 1/6881; C12Q 1/6874; C12Q 1/6844; C12Q 2600/158; C12Q 2600/16

(57) ABSTRACT

Disclosed herein, inter alia, are compositions, methods, and kits for sequencing nucleic acid molecules, such as immune receptor transcripts.

### CROSS-REFERENCE TO RELATED APPLICATIONS

This application is a continuation of International Application No. PCT/US2022/035022, filed Jun. 24, 2022, and which claims the benefit of U.S. Provisional Application No. 63/215,371, filed Jun. 25, 2021, each of which is incorporated herein by reference in their entirety and for all purposes.

### SEQUENCE LISTING

The instant application contains a Sequence Listing XML which has been submitted electronically and is hereby incorporated by reference in its entirety. Said Sequence Listing XML copy, created on Dec. 4, 2023, is named 551C01US Sequence Listing ST26.xml and is 8,582 bytes in size.

### BACKGROUND

The functions of immune cells such as B- and T-cells are predicated on the recognition through specialized receptors of specific targets (antigens) in pathogens. B-cell receptors (BCRs) are composed of variable heavy (VH) and variable light (VL) chains, and T-Cell receptors (TCRs) are composed of TCRα/γ and TCRβ/δ chains. Because the two chains of adaptive immune receptors are encoded by two independent transcripts, determining the BCR or TCR repertoire requires accurately determining the sequences of two independent polynucleotides from each cell. Despite many recent technical advances, determining the VH-VL or TCRβ-TCRα repertoire with high accuracy remains challenging.

### BRIEF SUMMARY

In view of the foregoing, innovative approaches to address issues with existing sequencing technologies are needed. Disclosed herein are solutions to these and other problems in the art.

In an aspect is provided a method of amplifying a tagged complement of two independent single-stranded polynucleotides, the method including: a. hybridizing a bridge oligonucleotide to a first polynucleotide and a second polynucleotide, thereby forming a bridged polynucleotide complex; b. hybridizing one or more interposing oligonucleotide probes to the first polynucleotide and second polynucleotide, wherein each of the interposing oligonucleotide probes includes from 5′ to 3′: i. a first hybridization sequence complementary to a first sequence of the first polynucleotide and second polynucleotide; ii. a loop region including a primer binding sequence and optionally a barcode; and iii. a second hybridization sequence complementary to a second sequence of first polynucleotide and second polynucleotide; c. extending the 3′ end of each second hybridization sequence of the interposing oligonucleotide probes and the 3′ end of the hybridization sequence of the bridge oligonucleotide with one or more polymerases thereby forming an extension product of each of the oligonucleotide probes; d. ligating the 3′ end of each of the extension products to the 5′ end of the adjacent extension products, thereby making an integrated strand including a complement of the template nucleic acid including a plurality of the oligonucleotide probes; and e. amplifying the integrated strand by an amplification reaction to produce a tagged complement of two independent single-stranded polynucleotides.

In an aspect is provided a method of amplifying a tagged complement of two independent single-stranded polynucleotides, the method including: a. hybridizing a first overlap oligonucleotide to the first independent polynucleotide and a second overlap oligonucleotide to the second independent polynucleotide, and extending both the first and second overlap oligonucleotides with a polymerase, thereby forming an overlapped polynucleotide complex, wherein the overlapped polynucleotide complex includes a complement of the first independent polynucleotide, the first overlap oligonucleotide, the second overlap oligonucleotide, and a complement of the second independent polynucleotide, wherein a 5′ sequence of the first overlap oligonucleotide is hybridized to a 5′ sequence of the second overlap oligonucleotide; b. linking the overlapped polynucleotide complex, thereby generating a bridged polynucleotide including a sequence of the first independent polynucleotide linked to a sequence of the second independent polynucleotide or a complement thereof, c. hybridizing to the bridged polynucleotide one or more interposing oligonucleotide barcodes, wherein each of the interposing oligonucleotide barcodes includes from 5′ to 3′: i. a first hybridization sequence complementary to a first sequence of the bridged polynucleotide; ii. a loop region including a barcode; and iii. a second hybridization sequence complementary to a second sequence of the bridged polynucleotide; d. extending the 3′ end of each second hybridization sequence of the interposing oligonucleotide barcodes with one or more polymerases thereby forming an extension product of each of the interposing oligonucleotide barcodes; e. ligating the 3′ end of each of the extension products to the 5′ end of the adjacent extension products hybridized to the bridged polynucleotide thereby making an integrated strand tagged with a plurality of interposing oligonucleotide barcodes, wherein the integrated strand includes sequences of the first and second independent polynucleotides or complements thereof; and f. amplifying the integrated strand by an amplification reaction to produce a tagged complement of two independent single-stranded polynucleotides.

In an aspect is provided a bridged polynucleotide including a complement of a first independent single-stranded polynucleotide sequence, a bridging oligonucleotide sequence, a complement of a second independent single-stranded polynucleotide sequence, and a plurality of interposing oligonucleotide barcode adapters.

In an aspect is provided a method of forming an integrated strand complement of a bridged polynucleotide including a plurality of oligonucleotide probes, wherein the bridged polynucleotide includes a complement of two independent single-stranded polynucleotides, the method including: a. hybridizing a bridge oligonucleotide to a first independent polynucleotide and a second independent polynucleotide, thereby forming a bridged polynucleotide complex; b. amplifying the bridged polynucleotide complex, thereby generating a bridged polynucleotide including a sequence of the first independent polynucleotide linked to a sequence of the second independent polynucleotide, or a complement thereof, c. hybridizing one or more interposing oligonucleotide probes to the bridged polynucleotide, hybridizing a 5′ terminal oligonucleotide probe downstream of the one or more interposing oligonucleotide probes, and hybridizing a 3′ terminal oligonucleotide probe upstream of the one or more interposing oligonucleotide probes, wherein each of the interposing oligonucleotide probes includes from 5′ to 3′: i. a first hybridization sequence complementary to a first sequence of the bridged polynucleotide; ii. a loop region including a primer binding sequence; and iii. a second hybridization sequence complementary to a second sequence of the bridged polynucleotide; wherein the 5′ terminal oligonucleotide probe includes from 5′ to 3′: i. a hybridization sequence complementary to a third sequence of the bridged polynucleotide; and ii. a primer binding sequence; and wherein the 3′ terminal oligonucleotide probe includes from 3′ to 5′: i. a hybridization sequence complementary to a fourth sequence of the bridged polynucleotide; and ii. a primer binding sequence; d. extending the 3′ end of each second hybridization sequence of the interposing oligonucleotide probes and the 3′ end of the hybridization sequence of the 3′ terminal oligonucleotide probe with one or more polymerases thereby forming an extension product of each of the oligonucleotide probes; e. ligating the 3′ end of each of the extension products to the 5′ end of the adjacent extension products, and ligating the 5′ end of the 5′ terminal oligonucleotide probe to the 3′ end of the adjacent extension product, each hybridized to the same bridged polynucleotide thereby making an integrated strand including a complement of the bridged polynucleotide including a plurality of the oligonucleotide probes; and f. amplifying the integrated strand by an amplification reaction to produce a complement of the integrated strand thereby forming an integrated strand complement of the bridged polynucleotide including oligonucleotide probes, wherein the complement of the integrated strand includes a complement of the plurality of oligonucleotide probes.

In an aspect is provided bridged polynucleotide including a complement of a first independent single-stranded polynucleotide sequence, a bridging oligonucleotide sequence, a complement of a second independent single-stranded polynucleotide sequence, and a plurality of interposing oligonucleotide probes.

In an aspect is provided a kit including: i. a plurality of interposing oligonucleotide probes capable of hybridizing to a bridged polynucleotide, the interposing oligonucleotide probes including from 5′ to 3′: i. a first hybridization sequence complementary to a first sequence of the bridged polynucleotide; ii. a loop region including a primer binding sequence; and iii. a second hybridization sequence complementary to a second sequence of the bridged polynucleotide; ii. a plurality of 5′ terminal oligonucleotide probes capable of hybridizing to a bridged polynucleotide, the 5′ terminal oligonucleotide probes including from 5′ to 3′: i. a hybridization sequence complementary to a 5′ terminal sequence of the bridged polynucleotide, wherein the 5′ terminal sequence is upstream of the bridged polynucleotide sequence complementary to the interposing oligonucleotide probes; and ii. a primer binding sequence; iii. a plurality of 3′ terminal oligonucleotide probes capable of hybridizing to a bridged polynucleotide, the 3′ terminal oligonucleotide probes including from 3′ to 5′: i. a hybridization sequence complementary to a 3′ terminal sequence of the bridged polynucleotide, wherein the 3′ terminal sequence is downstream of the bridged polynucleotide sequence complementary to the interposing oligonucleotide probes; and ii. a primer binding sequence; and iv. a bridging oligonucleotide including from 5′ to 3′: i. a hybridization sequence complementary to a 3′ terminal sequence of a first independent polynucleotide; ii. a linker sequence; and iii. a hybridization sequence complementary to a 5′ terminal sequence of a second independent polynucleotide.

### BRIEF DESCRIPTION OF THE DRAWINGS

FIGS. 1A-1B illustrate interposing barcodes (IBC) as described herein. FIG. 1A is an overview of a non-limiting example of an interposing barcode showing Type 1 and Type 2 IBCs, wherein Type 2 includes an additional identifying region (e.g., sample barcode, such as a 4 to 5 nucleotide section used to identify the sample, also referred to as a “sample index sequence”). Depending on the experiment, both Type 1 and Type 2 may be used. FIG. 1B shows an interposing barcode subjected to denaturing conditions (i.e. the stem regions are no longer hybridized together).

FIGS. 2A-2C illustrates a sequencing process, in accordance with an embodiment described herein. FIG. 2A depicts a single strand genomic DNA, to which a plurality of interposing barcodes are hybridized. A polymerase extends (depicted as the gray, cloud-like, structure) from the 3′ end of an interposing barcode and halts extension at or around the next interposing barcode. Dashed lines represent yet-to-be extension sites. A ligase (not shown) then ligates the extension strands and interposing barcodes together to produce a long, continuous DNA strand which contains integrated barcodes, as shown in FIG. 2B. When the hairpins stems are not hybridized together, the resultant single strand is shown in FIG. 2C. Note, the shading used in the figures is not indicative of an identical sequence. For example, despite the loops depicted in FIG. 2A are rendered in the same color/shading, this does not imply the sequences of the loops are identical. As described herein, the only sequences that are common are the stems of the interposing barcodes.

FIG. 3 depicts sequenced strands assembled into contiguous long reads by aligning the IBCs. Shown in the dashed box are instances where two IBCs are present on a single read, thus allowing greater information on the location and origin of the genomic input. The last read shows a complete IBC and a partial IBC on the lower right, conceptually depicting how utilizing embodiments of compositions and methods described herein provide a scaffold for the underlying genomic input.

FIG. 4 illustrates an alternative IBC wherein the hybridization sequences are asymmetric. As described further within the application, the 5′ hybridization sequence is elongated relative to the 3′ hybridization sequence possessing a 5′ flap (the raised portion of the hybridization sequence) for use with FEN1. This IBC may be Type 1 or Type 2, though the additional barcode is not shown in this depiction.

FIGS. 5A-5C demonstrate potential RNA workflow options as further described in Example 3.

FIGS. 6A-6C illustrate a single-cell B-cell receptor heavy-chain (HC) and light-chain (LC) mRNA isolation protocol.

FIGS. 7A-7B provide workflow examples for an embodiment of HC and LC library preparation using a bridge oligo and IBCs: IgG HC and LC mRNA is hybridized to IBCs and a barcoded bridge oligo that captures both the HC and LC mRNA (FIG. 7A). Hybridization produces a bridged polynucleotide, composed of the two mRNA molecules linked by hybridization to the bridge oligo. Following IBC and bridge oligo hybridization, RT-PCR is performed, followed by ligation to generate a contiguous molecule (FIG. 7B; the black bar denotes the cDNA product). Following amplicon production, droplets are broken and full-length paired HC-LC cDNA is isolated and prepared for sequencing. For example, the isolated ligation product (i.e., the integrated strand) is fragmented. Following fragmentation, the fragments are end repaired or end polished. Additional sequences such as adapters or primers may then be added to permit platform specific sequences or to provide a binding site for sequencing primers. Following adapter ligation, the nucleic acid templates may be purified, amplified, or sequenced using methods known to those skilled in the art.

FIGS. 8A-8B illustrate embodiments of bridge oligonucleotides as described herein. The bridge oligo may include two hybridization sequences consisting of targeted priming regions flanking a linking region (type 1) (FIG. 8A). Alternatively, the bridge oligo may include two hybridization sequences consisting of targeted priming regions flanking a fixed barcode region that is 5′ to a random barcode region (type 2) (FIG. 8B).

FIGS. 9A-9C provide workflow examples for another embodiment of HC and LC library preparation using hybridized overlap oligonucleotides (e.g., a first overlap oligonucleotide and a second overlap oligonucleotide, wherein a sequence at the 5′ end of the first overlap oligonucleotide is hybridized to a sequence at the 5′ end of the second overlap oligonucleotide) and IBCs. IgG HC and LC mRNA are annealed to two hybridized overlap oligonucleotides followed by reverse transcription (e.g., reverse transcription in overlap extension RT-PCR), wherein each overlap oligonucleotide is specific for the variable region of the IgG HC or IgG LC mRNA (FIG. 9A). The cDNA strands are illustrated as black lines to distinguish from the mRNA strands. Following reverse transcription, second strand cDNA synthesis is performed (e.g., RNAse H nicking followed by DNA Polymerase I extension and ligation of the products to form a contiguous cDNA strand). The reverse transcription and second strand cDNA synthesis steps produce a double-stranded bridged polynucleotide (e.g., a double-stranded cDNA bridged polynucleotide) having cDNA sequences of the two mRNA molecules, covalently linked by sequences of the two overlap oligonucleotides. PCR enrichment of the cDNA product may optionally be performed using forward and reverse primers (FIG. 9B). Following overlap-extension RT-PCR, full-length, single-stranded paired HC-LC cDNA is isolated. To this isolated cDNA sample, IBCs (as described herein) are added at an appropriate concentration such that there are approximately 50-200 bases between each IBC. A non strand-displacing polymerase (e.g., Klentaq, T4, T7, Bst, Phusion, Tfl, Pfu, or Stoffel fragment) extends the complement strand to generate an extension segment, as shown in FIG. 2A, and a ligase ligates the ends of the extension segment together with the next interposing barcode to produce a single integrated strand (FIG. 9C).

FIG. 10 describes a non-limiting example of the methods described herein. As described herein, a plurality of interposing barcodes (IBCs), are hybridized to a sample polynucleotide, extended, and ligated together to form a tagged complement of the sample polynucleotide. The IBCs are represented as A, B, C, D, E, and F in FIG. 10. The tagged complement is then amplified (step 2 of FIG. 10) and fragmented. The fragments may be prepared according to standard library prep methods (e.g., polishing, A-tailing, etc.) and have platform specific primers/adapters ligated to the ends to make them compatible with particular sequencing modalities. The fragments are then sequenced and the barcodes are identified for each sequencing read. The sequencing reads are grouped according the co-occurrence of IBCs, and within each group all the sequencing reads containing a group member are identified and assembled.

FIG. 11 illustrates an embodiment wherein IBCs are hybridized to a template polynucleotide in combination with terminal adapters. In embodiments, the terminal adapters include one or two hybridization sequences as described herein, a barcode (e.g., a UMI), and a primer binding sequence.

FIGS. 12A-12D illustrate interposing probes (IPP) as described herein. FIG. 12A is an overview of a non-limiting example of an interposing probe. In embodiments, the interposing probe consists of the hybridization sequences and loop (e.g., an IPP without the stem regions). FIG. 12B shows an interposing probe subjected to denaturing conditions (i.e., the stem regions are no longer hybridized together). FIG. 12C shows a 3′ flanking adapter, alternatively referred to herein as a 3′ terminal oligonucleotide probe, including a P1 primer binding sequence on the 5′ end and a hybridization sequence on the 3′ end. FIG. 12D shows a 5′ flanking adapter, alternatively referred to herein as a 5′ terminal oligonucleotide probe, including a P2 primer binding sequence on the 3′ end and a hybridization sequence on the 5′ end.

FIG. 13 illustrates an amplification process integrating the interposing probes (IPPs) as described herein to form an integrated strand. Depicted in the top panel is a single-stranded template DNA molecule (e.g., a bridged polynucleotide including two independent polynucleotides linked together as described in FIGS. 7A-7B or FIGS. 9A-9C), to which a plurality of IPPs and two flanking adapters are hybridized (e.g., IPPs and flanking adapters as described in FIGS. 12A-12D). Next, a polymerase (depicted as a cloud-like object) extends from the 3′ end of each hybridized IPP and 3′ terminal adapter and halts extension at or around the next IPP or flanking adapter. A ligase (not shown) then ligates the extended strands, IPPs, and flanking adapters together to produce a long, continuous DNA strand which contains integrated probes and complements of the original template DNA molecule. When the hairpin stems are not hybridized together, the resultant single-stranded polynucleotide is shown in the bottom panel. Note, the shading/coloring used in the figures is not indicative of an identical sequence. For example, although the loops depicted in the top panel of FIG. 13 are rendered in the same color/shading, this does not imply the sequences of the loops are identical. In embodiments, the only sequences that are common are the stems of the interposing probes.

FIGS. 14A-14C illustrate an embodiment of a sequencing process of an interposing probe-containing single-stranded polynucleotide immobilized on a substrate. FIG. 14A shows hybridization of a first sequencing primer (e.g., SP1) to the flanking adapter on the 3′ end of the DNA strand and extension in the presence of a polymerase and detectable nucleotides (shown as a star) the first region of the polynucleotide. Next, sequencing is terminated through the incorporation of a blocking element, for example, a ddNTP (shown here as an octagon). A second sequencing primer (e.g., SP2) is then hybridized to a sequence of the first interposing probe (e.g., the loop portion), and a second region of the polynucleotide is sequenced in the presence of a polymerase and detectable nucleotides, followed by termination of extension through incorporation of another blocking element (e.g., a ddNTP). As shown in FIG. 14B, a third sequencing primer (e.g., SP3) is then hybridized to a sequence of the second interposing probe (e.g., the loop portion), and a third region of the polynucleotide is sequenced in the presence of a polymerase and detectable nucleotides. Sequencing of the third region is then terminated through incorporation of a third blocking element (e.g., a ddNTP). A fourth sequencing primer is then hybridized to a sequence of the third interposing probe (e.g., the loop portion), and a fourth region (e.g., the 5′ end) of the polynucleotide is sequenced in the presence of a polymerase and detectable nucleotides. In embodiments, the sequencing primer hybridizes to the loop region, the stem region, or the hybridization sequence region. In embodiments, the sequencing primer hybridizes to the loop region. In embodiments, the sequencing primer hybridizes to the stem region (e.g., one of the two complementary stem regions of the IPP). In embodiments, the sequencing primer hybridizes to the hybridization sequence (e.g., one of the two hybridization sequence regions of the IPP). Sequencing of the fourth region is then terminated through incorporation of a fourth blocking element (e.g., ddNTP). While each sequencing primer and corresponding sequenced region of the template polynucleotide are illustrated as being spaced in regular intervals, it is understood that each sequenced region may be of varying lengths, and sequencing primers may be targeted to non-adjacent portions of the template polynucleotide. FIG. 14C illustrates the process of bioinformatically reconstructing and aligning variable-length sequencing reads based on known features, e.g., sequencing primer binding sequences. In this example, reads with matching features are surrounded by a dashed box.

### DETAILED DESCRIPTION

The aspects and embodiments described herein relate to compositions and methods for sequencing the immune receptor repertoires at significant depth with a high level of precision, enabling, for example, the study of tumor-infiltrating lymphocytes for adaptive TCR therapy and further development in vaccine responses.

### I. Definitions

The practice of the technology described herein will employ, unless indicated specifically to the contrary, conventional methods of chemistry, biochemistry, organic chemistry, molecular biology, bioinformatics, microbiology, recombinant DNA techniques, genetics, immunology, and cell biology that are within the skill of the art, many of which are described below for the purpose of illustration. Examples of such techniques are available in the literature. See, e.g., Singleton et al., DICTIONARY OF MICROBIOLOGY AND MOLECULAR BIOLOGY 2nd ed., J. Wiley & Sons (New York, NY 1994); and Sambrook and Green, Molecular Cloning: A Laboratory Manual, 4th Edition (2012). Methods, devices and materials similar or equivalent to those described herein can be used in the practice of this invention. All patents, patent applications, articles and publications mentioned herein, both supra and infra, are hereby expressly incorporated herein by reference in their entireties.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present.

As used herein, the term “control” or “control experiment” is used in accordance with its plain and ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

As used herein, the term “associated” or “associated with” can mean that two or more species are identifiable as being co-located at a point in time. An association can mean that two or more species are or were within a similar container. An association can be an informatics association, where for example digital information regarding two or more species is stored and can be used to determine that one or more of the species were co-located at a point in time. An association can also be a physical association.

As used herein, the term “contacting” is used in accordance with its plain ordinary meaning and refers to the process of allowing at least two distinct species (e.g. chemical compounds including biomolecules or cells) to become sufficiently proximal to react, interact or physically touch. However, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a compound, nucleic acid, a protein, or enzyme (e.g., a DNA polymerase).

As used herein, the term “nucleic acid” is used in accordance with its plain and ordinary meaning and refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides include single and double stranded DNA, single and double stranded RNA, and hybrid molecules having mixtures of single and double stranded DNA and RNA with linear or circular framework. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may include natural nucleic acid sequences and variants thereof, artificial nucleic acid sequences, or a combination of such sequences. A “nucleoside” is structurally similar to a nucleotide, but is missing the phosphate moieties. An example of a nucleoside analogue would be one in which the label is linked to the base and there is no phosphate group attached to the sugar molecule. As may be used herein, the terms “nucleic acid oligomer” and “oligonucleotide” are used interchangeably and are intended to include, but are not limited to, nucleic acids having a length of 200 nucleotides or less. In some embodiments, an oligonucleotide is a nucleic acid having a length of 2 to 200 nucleotides, 2 to 150 nucleotides, 5 to 150 nucleotides or 5 to 100 nucleotides. The terms “polynucleotide,” “oligonucleotide,” “oligo” or the like refer, in the usual and customary sense, to a linear sequence of nucleotides. Oligonucleotides are typically from about 5, 6, 7, 8, 9, 10, 12, 15, 25, 30, 40, 50 or more nucleotides in length, up to about 100 nucleotides in length. In some embodiments, an oligonucleotide is a primer configured for extension by a polymerase when the primer is annealed completely or partially to a complementary nucleic acid template. A primer is often a single stranded nucleic acid. In certain embodiments, a primer, or portion thereof, is substantially complementary to a portion of an adapter. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In some embodiments, an oligonucleotide may be immobilized to a solid support.

As used herein, the term “independent polynucleotide” refers to a first polynucleotide (e.g., a first independent polynucleotide) that is different in sequence from a second polynucleotide (e.g., a second independent polynucleotide). In embodiments, the one or more independent polynucleotides are present in the same sample. In embodiments, the one or more independent polynucleotides are present in the same cell. The first and second independent polynucleotides may be linked together, for example, with a bridging oligonucleotide or at least two overlap oligonucleotides as described herein. In embodiments, the first independent polynucleotide and the second independent polynucleotide are functionally related. For example, the first independent polynucleotide is a mRNA molecule encoding an IgG heavy chain transcript, and the second independent polynucleotide in a mRNA molecule encoding an IgG light chain transcript. Following linking with a bridging oligonucleotide or overlap oligonucleotides, for example, the bridged polynucleotide would include both the IgG heavy chain and IgG light chain sequences.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As used herein, the term “template nucleic acid” refers to any polynucleotide molecule that may be bound by a polymerase and utilized as a template for nucleic acid synthesis. A template nucleic acid may also be referred to herein as a “template polynucleotide”. A template nucleic acid may be a target nucleic acid. A template polynucleotide may be a target polynucleotide. In general, the term “target nucleic acid” refers to a nucleic acid molecule or polynucleotide in a starting population of nucleic acid molecules having a target sequence whose presence, amount, and/or nucleotide sequence, or changes in one or more of these, are desired to be determined. In general, the term “target sequence” refers to a nucleic acid sequence on a single strand of nucleic acid. The terms “single strand” and “ssDNA” are used in accordance with its plain and ordinary meaning and refer to a single-stranded polynucleotide. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction. A target nucleic acid is not necessarily any single molecule or sequence. For example, a target nucleic acid may be any one of a plurality of target nucleic acids in a reaction, or all nucleic acids in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. As yet another example, all or a subset of polynucleotides in a sample may be modified by the addition of a primer-binding sequence (such as by the ligation of adapters containing the primer binding sequence), rendering each modified polynucleotide a target nucleic acid in a reaction with the corresponding primer polynucleotide(s). In the context of selective sequencing, “target nucleic acid(s)” refers to the subset of nucleic acid(s) to be sequenced from within a starting population of nucleic acids.

In embodiments, a target polynucleotide is a cell-free polynucleotide. In general, the terms “cell-free,” “circulating,” and “extracellular” as applied to polynucleotides (e.g. “cell-free DNA” (cfDNA) and “cell-free RNA” (cfRNA)) are used interchangeably to refer to polynucleotides present in a sample from a subject or portion thereof that can be isolated or otherwise manipulated without applying a lysis step to the sample as originally collected (e.g., as in extraction from cells or viruses). Cell-free polynucleotides are thus unencapsulated or “free” from the cells or viruses from which they originate, even before a sample of the subject is collected. Cell-free polynucleotides may be produced as a byproduct of cell death (e.g. apoptosis or necrosis) or cell shedding, releasing polynucleotides into surrounding body fluids or into circulation. Accordingly, cell-free polynucleotides may be isolated from a non-cellular fraction of blood (e.g. serum or plasma), from other bodily fluids (e.g. urine), or from non-cellular fractions of other types of samples.

As used herein, a “native” nucleotide is used in accordance with its plain and ordinary meaning and refers to a naturally occurring nucleotide that does not include an exogenous label (e.g., a fluorescent dye, or other label) or chemical modification such as may characterize a nucleotide analog (e.g., a reversible terminating moiety). Examples of native nucleotides useful for carrying out procedures described herein include: dATP (2′-deoxyadenosine-5′-triphosphate); dGTP (2′-deoxyguanosine-5′-triphosphate); dCTP (2′-deoxycytidine-5′-triphosphate); dTTP (2′-deoxythymidine-5′-triphosphate); and dUTP (2′-deoxyuridine-5′-triphosphate). A “canonical” nucleotide is an unmodified nucleotide.

A nucleic acid can be amplified by a suitable method. The term “amplified” as used herein refers to subjecting a target nucleic acid in a sample to a process that linearly or exponentially generates amplicon nucleic acids having the same or substantially the same (e.g., substantially identical) nucleotide sequence as the target nucleic acid, or segment thereof, and/or a complement thereof. In some embodiments an amplification reaction includes a suitable thermal stable polymerase. Thermal stable polymerases are known in the art and are stable for prolonged periods of time, at temperature greater than 80° C. when compared to common polymerases found in most mammals. In certain embodiments the term “amplified” refers to a method that includes a polymerase chain reaction (PCR). Conditions conducive to amplification (i.e., amplification conditions) are known and often include at least a suitable polymerase, a suitable template, a suitable primer or set of primers, suitable nucleotides (e.g., dNTPs), a suitable buffer, and application of suitable annealing, hybridization and/or extension times and temperatures. In certain embodiments an amplified product (e.g., an amplicon) can contain one or more additional and/or different nucleotides than the template sequence, or portion thereof, from which the amplicon was generated (e.g., a primer can contain “extra” nucleotides (such as a 5′ portion that does not hybridize to the template), or one or more mismatched bases within a hybridizing portion of the primer).

A nucleic acid can be amplified by a thermocycling method or by an isothermal amplification method. In some embodiments, a rolling circle amplification method is used. In some embodiments, amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support.

In some embodiments solid phase amplification includes a nucleic acid amplification reaction including only one species of oligonucleotide primer immobilized to a surface or substrate. In certain embodiments solid phase amplification includes a plurality of different immobilized oligonucleotide primer species. In some embodiments solid phase amplification may include a nucleic acid amplification reaction including one species of oligonucleotide primer immobilized on a solid surface and a second different oligonucleotide primer species in solution. Multiple different species of immobilized or solution-based primers can be used. Non-limiting examples of solid phase nucleic acid amplification reactions include interfacial amplification, bridge amplification, emulsion PCR, WildFire amplification (e.g., US patent publication US20130012399), the like or combinations thereof.

In embodiments, a target nucleic acid is a cell-free nucleic acid. In general, the terms “cell-free,” “circulating,” and “extracellular” as applied to nucleic acids (e.g. “cell-free DNA” (cfDNA) and “cell-free RNA” (cfRNA)) are used interchangeably to refer to nucleic acids present in a sample from a subject or portion thereof that can be isolated or otherwise manipulated without applying a lysis step to the sample as originally collected (e.g., as in extraction from cells or viruses). Cell-free nucleic acids are thus unencapsulated or “free” from the cells or viruses from which they originate, even before a sample of the subject is collected. Cell-free nucleic acids may be produced as a byproduct of cell death (e.g. apoptosis or necrosis) or cell shedding, releasing nucleic acids into surrounding body fluids or into circulation. Accordingly, cell-free nucleic acids may be isolated from a non-cellular fraction of blood (e.g. serum or plasma), from other bodily fluids (e.g. urine), or from non-cellular fractions of other types of samples.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site blast.ncbi.nlm.nih.gov/Blast.cgi or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The term “messenger RNA” or “mRNA” refers to an RNA that is without introns and is capable of being translated into a polypeptide. The term “RNA” refers to any ribonucleic acid, including but not limited to mRNA, tRNA (transfer RNA), rRNA (ribosomal RNA), and/or noncoding RNA (such as lncRNA (long noncoding RNA)). The term “cDNA” refers to a DNA that is complementary or identical to an RNA, in either single stranded or double stranded form.

As used herein, the terms “analogue” and “analog”, in reference to a chemical compound, refers to compound having a structure similar to that of another one, but differing from it in respect of one or more different atoms, functional groups, or substructures that are replaced with one or more other atoms, functional groups, or substructures. In the context of a nucleotide, a nucleotide analog refers to a compound that, like the nucleotide of which it is an analog, can be incorporated into a nucleic acid molecule (e.g., an extension product) by a suitable polymerase, for example, a DNA polymerase in the context of a nucleotide analogue. The terms also encompass nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, e.g., Eckstein, OLIGONUCLEOTIDES AND ANALOGUES: A PRACTICAL APPROACH, Oxford University Press) as well as modifications to the nucleotide bases such as in 5-methyl cytidine or pseudouridine.; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds.) Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

As used herein, the term “modified nucleotide” refers to nucleotide modified in some manner. Typically, a nucleotide contains a single 5-carbon sugar moiety, a single nitrogenous base moiety and 1 to three phosphate moieties. In embodiments, a nucleotide can include a blocking moiety (alternatively referred to herein as a reversible terminator moiety) and/or a label moiety. A blocking moiety on a nucleotide prevents formation of a covalent bond between the 3′ hydroxyl moiety of the nucleotide and the 5′ phosphate of another nucleotide. A blocking moiety on a nucleotide can be reversible, whereby the blocking moiety can be removed or modified to allow the 3′ hydroxyl to form a covalent bond with the 5′ phosphate of another nucleotide. A blocking moiety can be effectively irreversible under particular conditions used in a method set forth herein. In embodiments, the blocking moiety is attached to the 3′ oxygen of the nucleotide and is independently —NH₂, —CN, —CH₃, C₂-C₆ allyl (e.g., —CH₂—CH═CH₂), methoxyalkyl (e.g., —CH₂—O—CH₃), or —CH₂N₃. In embodiments, the blocking moiety is attached to the 3′ oxygen of the nucleotide and is independently

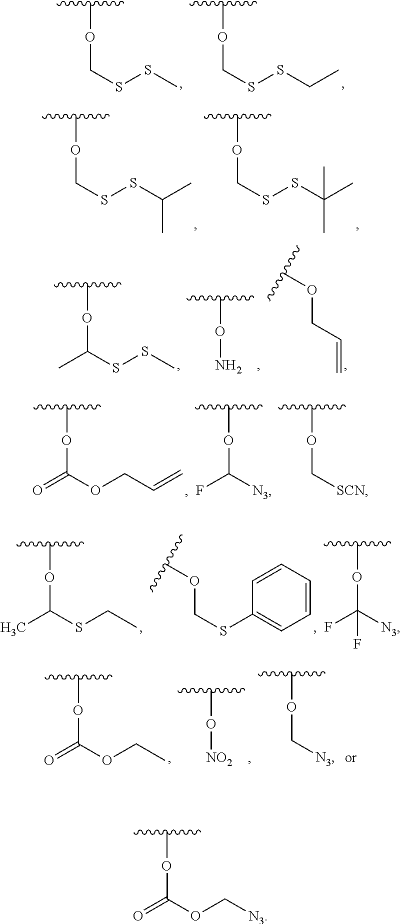

A label moiety of a nucleotide can be any moiety that allows the nucleotide to be detected, for example, using a spectroscopic method. Exemplary label moieties are fluorescent labels, mass labels, chemiluminescent labels, electrochemical labels, detectable labels and the like. One or more of the above moieties can be absent from a nucleotide used in the methods and compositions set forth herein. For example, a nucleotide can lack a label moiety or a blocking moiety or both. Examples of nucleotide analogues include, without limitation, 7-deaza-adenine, 7-deaza-guanine, the analogues of deoxynucleotides shown herein, analogues in which a label is attached through a cleavable linker to the 5-position of cytosine or thymine or to the 7-position of deaza-adenine or deaza-guanine, and analogues in which a small chemical moiety is used to cap the —OH group at the 3′-position of deoxyribose. Nucleotide analogues and DNA polymerase-based DNA sequencing are also described in U.S. Pat. No. 6,664,079, which is incorporated herein by reference in its entirety for all purposes.

The term “cleavable linker” or “cleavable moiety” as used herein refers to a divalent or monovalent, respectively, moiety which is capable of being separated (e.g., detached, split, disconnected, hydrolyzed, a stable bond within the moiety is broken) into distinct entities. A cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). A chemically cleavable linker refers to a linker which is capable of being split in response to the presence of a chemical (e.g., acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), or hydrazine (N₂H₄)). A chemically cleavable linker is non-enzymatically cleavable. In embodiments, the cleavable linker is cleaved by contacting the cleavable linker with a cleaving agent. In embodiments, the cleaving agent is a phosphine containing reagent (e.g., TCEP or THPP), sodium dithionite (Na₂S₂O₄), weak acid, hydrazine (N₂H₄), Pd(0), or light-irradiation (e.g., ultraviolet radiation). In embodiments, cleaving includes removing. A “cleavable site” or “scissile linkage” in the context of a polynucleotide is a site which allows controlled cleavage of the polynucleotide strand (e.g., the linker, the primer, or the polynucleotide) by chemical, enzymatic, or photochemical means known in the art and described herein. A scissile site may refer to the linkage of a nucleotide between two other nucleotides in a nucleotide strand (i.e., an internucleosidic linkage). In embodiments, the scissile linkage can be located at any position within the one or more nucleic acid molecules, including at or near a terminal end (e.g., the 3′ end of an oligonucleotide) or in an interior portion of the one or more nucleic acid molecules. In embodiments, conditions suitable for separating a scissile linkage include a modulating the pH and/or the temperature. In embodiments, a scissile site can include at least one acid-labile linkage. For example, an acid-labile linkage may include a phosphoramidate linkage. In embodiments, a phosphoramidate linkage can be hydrolysable under acidic conditions, including mild acidic conditions such as trifluoroacetic acid and a suitable temperature (e.g., 30° C.), or other conditions known in the art, for example Matthias Mag, et al Tetrahedron Letters, Volume 33, Issue 48, 1992, 7319-7322. In embodiments, the scissile site can include at least one photolabile internucleosidic linkage (e.g., o-nitrobenzyl linkages, as described in Walker et al, J. Am. Chem. Soc. 1988, 110, 21, 7170-7177), such as o-nitrobenzyloxymethyl or p-nitrobenzyloxymethyl group(s). In embodiments, the scissile site includes at least one uracil nucleobase. In embodiments, a uracil nucleobase can be cleaved with a uracil DNA glycosylase (UDG) or Formamidopyrimidine DNA Glycosylase Fpg. In embodiments, the scissile linkage site includes a sequence-specific nicking site having a nucleotide sequence that is recognized and nicked by a nicking endonuclease enzyme or a uracil DNA glycosylase.

As used herein, the term “complement” is used in accordance with its plain and ordinary meaning and refers to a nucleotide (e.g., RNA nucleotide or DNA nucleotide) or a sequence of nucleotides capable of base pairing with a complementary nucleotide or sequence of nucleotides. As described herein and commonly known in the art the complementary (matching) nucleotide of adenosine is thymidine in DNA, or alternatively in RNA the complementary (matching) nucleotide of adenosine is uracil, and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. The pairing of purine containing nucleotide (e.g., A or G) with a pyrimidine containing nucleotide (e.g., T or C) are considered complements. The A-T and C-G pairings function to form double or triple hydrogen bonds between the amine and carbonyl groups on the complementary bases. “Duplex” means at least two oligonucleotides and/or polynucleotides that are fully or partially complementary undergo Watson-Crick type base pairing among all or most of their nucleotides so that a stable complex is formed. Complementary single stranded nucleic acids and/or substantially complementary single stranded nucleic acids can hybridize to each other under hybridization conditions, thereby forming a nucleic acid that is partially or fully double stranded. When referring to a double-stranded polynucleotide including a first strand hybridized to a second strand, it is understood that each of the first strand and the second strand are independently single-stranded polynucleotides. All or a portion of a nucleic acid sequence may be substantially complementary to another nucleic acid sequence, in some embodiments.

As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often include nucleic acid sequences that are substantially complementary to each other.

As used herein, the term “removable” group, e.g., a label or a blocking group or protecting group, is used in accordance with its plain and ordinary meaning and refers to a chemical group that can be removed from a nucleotide analogue such that a DNA polymerase can extend the nucleic acid (e.g., a primer or extension product) by the incorporation of at least one additional nucleotide. Removal may be by any suitable method, including enzymatic, chemical, or photolytic cleavage. Removal of a removable group, e.g., a blocking group, does not require that the entire removable group be removed, only that a sufficient portion of it be removed such that a DNA polymerase can extend a nucleic acid by incorporation of at least one additional nucleotide using a nucleotide or nucleotide analogue. In general, the conditions under which a removable group is removed are compatible with a process employing the removable group (e.g., an amplification process or sequencing process).

As used herein, the terms “reversible blocking groups” and “reversible terminators” are used in accordance with their plain and ordinary meanings and refer to a blocking moiety located, for example, at the 3′ position of the nucleotide and may be a chemically cleavable moiety such as an allyl group, an azidomethyl group or a methoxymethyl group, or may be an enzymatically cleavable group such as a phosphate ester. Non-limiting examples of nucleotide blocking moieties are described in applications WO 2004/018497, U.S. Pat. Nos. 7,057,026, 7,541,444, WO 96/07669, U.S. Pat. Nos. 5,763,594, 5,808,045, 5,872,244 and 6,232,465 the contents of which are incorporated herein by reference in their entirety. The nucleotides may be labelled or unlabeled. They may be modified with reversible terminators useful in methods provided herein and may be 3′-O-blocked reversible or 3′-unblocked reversible terminators. In nucleotides with 3′-O-blocked reversible terminators, the blocking group —OR [reversible terminating (capping) group] is linked to the oxygen atom of the 3-OH of the pentose, while the label is linked to the base, which acts as a reporter and can be cleaved. The 3-O-blocked reversible terminators are known in the art, and may be, for instance, a 3′-ONH₂ reversible terminator, a 3-O-allyl reversible terminator, or a 3-O-azidomethyl reversible terminator. In embodiments, the reversible terminator moiety is

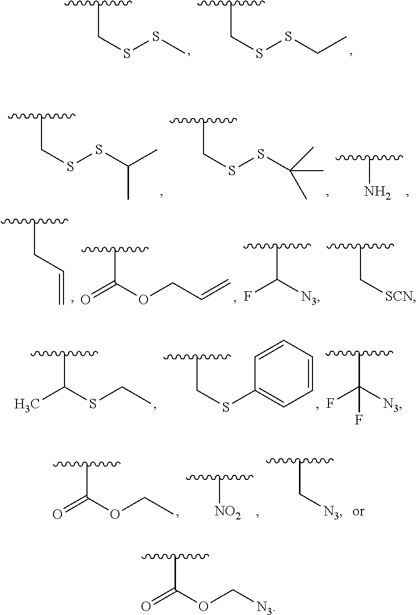

The term “allyl” as described herein refers to an unsubstituted methylene attached to a vinyl group (i.e., —CH═CH₂), having the formula

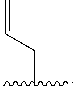 [SMILES: *CC=C]

In embodiments, the reversible terminator moiety is

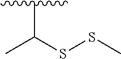 [SMILES: *C(C)SSC]

as described in U.S. Pat. No. 10,738,072, which is incorporated herein by reference for all purposes. For example, a nucleotide including a reversible terminator moiety may be represented by the formula:

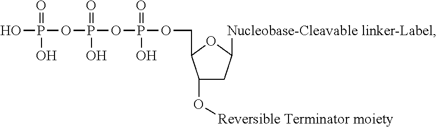

where the nucleobase is adenine or adenine analogue, thymine or thymine analogue, guanine or guanine analogue, or cytosine or cytosine analogue. In embodiments, the reversible terminator includes a hydrocarbyl. In embodiments, the reversible terminator includes an ester (O—C(O)R₁′ wherein R₁′ is any alkyl or aryl group which can include a formate, benzoyl formate, acetate, substituted acetate, propionate, and other esters as described in Green, T. W. (Protective Groups in Organic Chemistry, Wiley & Sons, New York, 1981)). In embodiments, the reversible terminator includes an ether (O—R₂′ wherein R₂′ can be substituted or unsubstituted alkyl such as methyl, substituted methyl, ethyl, substituted ethyl, allyl, substituted benzyl, silyl, or any other ether used to transiently protect hydroxyls and similar groups). In embodiments, the reversible terminator includes an O—CH₂(OC₂H₅)_{N′}CH₃ wherein N′ is an integer from 1-10. In embodiments, the reversible terminator includes a phosphate, phosphoramidate, phosphoramide, toluic acid ester, benzoic ester, acetic acid ester, or ethoxyethyl ether.

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity. In embodiments, sequences in a pair of complementary sequences form portions of a single polynucleotide with non-base-pairing nucleotides (e.g., as in a hairpin structure, with or without an overhang) or portions of separate polynucleotides. In embodiments, one or both sequences in a pair of complementary sequences form portions of longer polynucleotides, which may or may not include additional regions of complementarity.

The terms “bioconjugate group,” “bioconjugate reactive moiety,” and “bioconjugate reactive group” refer to a chemical moiety which participates in a reaction to form a bioconjugate linker (e.g., covalent linker). Non-limiting examples of bioconjugate reactive groups and the resulting bioconjugate reactive linkers may be found in the Bioconjugate Table below:

| Bioconjugate reactive group 1 | Bioconjugate reactive group 2 | |
|---|---|---|
| (e.g., electrophilic bioconjugate | (e.g., nucleophilic bioconjugate | Resulting Bioconjugate |
| reactive moiety) | reactive moiety) | reactive linker |
| activated esters | amines/anilines | carboxamides |
| acrylamides | thiols | thioethers |
| acyl azides | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carbodiimides | carboxylic acids | N-acylureas or anhydrides |
| diazoalkanes | carboxylic acids | esters |
| epoxides | thiols | thioethers |
| haloacetamides | thiols | thioethers |
| haloplatinate | amino | platinum complex |
| haloplatinate | heterocycle | platinum complex |
| haloplatinate | thiol | platinum complex |
| halotriazines | amines/anilines | aminotriazines |
| halotriazines | alcohols/phenols | triazinyl ethers |
| halotriazines | thiols | triazinyl thioethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | phenols/alcohols | sulfonate esters |

As used herein, the term “bioconjugate” or “bioconjugate linker” refers to the resulting association between atoms or molecules of bioconjugate reactive groups. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine). In embodiments, the first bioconjugate reactive group (e.g., —COOH) is covalently attached to the second bioconjugate reactive group (e.g.,

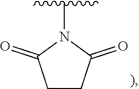 [SMILES: *N1C(=O)CCC1=O]

thereby forming a bioconjugate (e.g.,

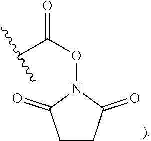 [SMILES: *C(=O)ON1C(=O)CCC1=O]

In embodiments, the first bioconjugate reactive group (e.g., —NH₂) is covalently attached to the second bioconjugate reactive group (e.g.,

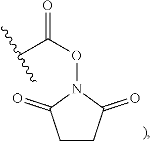 [SMILES: *C(=O)ON1C(=O)CCC1=O]

thereby forming a bioconjugate (e.g.,

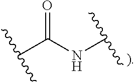 [SMILES: *NC(*)=O]

In embodiments, the first bioconjugate reactive group (e.g., a coupling reagent) is covalently attached to the second bioconjugate reactive group (e.g.,

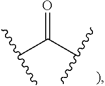 [SMILES: *C(*)=O]

thereby forming a bioconjugate (e.g.,

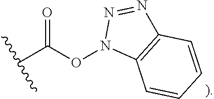 [SMILES: *C(=O)On1nnc2ccccc21]

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate includes a molecular entity derived from the reaction of an unsaturated bond, such as a maleimide, and a sulfhydryl group.

Useful bioconjugate reactive groups used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; (o) biotin conjugate can react with avidin or streptavidin to form a avidin-biotin complex or streptavidin-biotin complex.

The term “covalent linker” is used in accordance with its ordinary meaning and refers to a divalent moiety which connects at least two moieties to form a molecule. The term “non-covalent linker” is used in accordance with its ordinary meaning and refers to a divalent moiety which includes at least two molecules that are not covalently linked to each other but are capable of interacting with each other via a non-covalent bond (e.g., electrostatic interactions (e.g., ionic bond, hydrogen bond, halogen bond) or van der Waals interactions (e.g., dipole-dipole, dipole-induced dipole, London dispersion). In embodiments, the non-covalent linker is the result of two molecules that are not covalently linked to each other that interact with each other via a non-covalent bond.

The term “adapter” as used herein refers to any linear oligonucleotide that can be ligated to a nucleic acid molecule, thereby generating nucleic acid products that can be sequenced on a sequencing platform (e.g., an Illumina or Singular Genomics G4™ sequencing platform). In embodiments, adapters include two reverse complementary oligonucleotides forming a double-stranded structure. In embodiments, an adapter includes two oligonucleotides that are complementary at one portion and mismatched at another portion, forming a Y-shaped or fork-shaped adapter that is double stranded at the complementary portion and has two overhangs at the mismatched portion. Since Y-shaped adapters have a complementary, double-stranded region, they can be considered a special form of double-stranded adapters. When this disclosure contrasts Y-shaped adapters and double stranded adapters, the term “double-stranded adapter” or “blunt-ended” is used to refer to an adapter having two strands that are fully complementary, substantially (e.g., more than 90% or 95%) complementary, or partially complementary. In embodiments, adapters include sequences that bind to sequencing primers. In embodiments, adapters include sequences that bind to immobilized oligonucleotides (e.g., P7 and P5 sequences) or reverse complements thereof. In embodiments, the adapter is substantially non-complementary to the 3′ end or the 5′ end of any target polynucleotide present in the sample. In embodiments, the adapter can include a sequence that is substantially identical, or substantially complementary, to at least a portion of a primer, for example a universal primer. In embodiments, the adapter can include an index sequence (also referred to as barcode or tag) to assist with downstream error correction, identification or sequencing.

As used herein, the terms “hybridization” and “hybridizing” refer to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner according to base complementarity. The complex may include two strands forming a duplex structure, three or more strands forming a multi-stranded complex, a single self-hybridizing strand, or any combination of these. A hybridization reaction may constitute a step in a more extensive process, such as the initiation of PCR, or the enzymatic cleavage of a polynucleotide by an endonuclease. A second sequence that is perfectly complementary to a first sequence, or is polymerized by a polymerase using the first sequence as template, is referred to as “the complement” of the first sequence. The term “hybridizable” as applied to a polynucleotide refers to the ability of the polynucleotide to form a complex that is stabilized via hydrogen bonding between the bases of the nucleotide residues in a hybridization reaction. In some embodiments, a hybridizable sequence of nucleotides is at least about 50%, 60%, 70%, 75%, 80%, 85%, 90%, 95%, or 100% complementary to the sequence to which it hybridizes. In some embodiments, a hybridizable sequence is one that hybridizes to one or more target sequences as part of, and under the conditions of, a step in a multi-step process (e.g., a ligation reaction, or an amplification reaction). The propensity for hybridization between nucleic acids depends on the temperature and ionic strength of their milieu, the length of the nucleic acids and the degree of complementarity. The effect of these parameters on hybridization is described in, for example, Sambrook J., Fritsch E. F., Maniatis T., Molecular cloning: a laboratory manual, Cold Spring Harbor Laboratory Press, New York (1989). As used herein, hybridization of a primer, or of a DNA extension product, respectively, is extendable by creation of a phosphodiester bond with an available nucleotide or nucleotide analogue capable of forming a phosphodiester bond, therewith. For example, hybridization can be performed at a temperature ranging from 15° C. to 95° C. In some embodiments, the hybridization is performed at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C. In other embodiments, the stringency of the hybridization can further altered by the addition or removal of components of the buffered solution. A specific hybridization discriminates over non-specific hybridization interactions (e.g., two nucleic acids that a not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands that are hybridized to each other can form a duplex which includes a double-stranded portion of nucleic acid. The terms “hybridize” and “anneal”, and grammatical variations thereof, are used interchangeably herein. In some embodiments nucleic acids, or portions thereof, that are configured to specifically hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence.

In some embodiments, a nucleic acid includes a label. As used herein, the term “label” or “labels” are used in accordance with their plain and ordinary meanings and refer to molecules that can directly or indirectly produce or result in a detectable signal either by themselves or upon interaction with another molecule. Non-limiting examples of detectable labels include fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the label is a dye. In embodiments, the dye is a fluorescent dye. Non-limiting examples of dyes, some of which are commercially available, include CF dyes (Biotium, Inc.), Alexa Fluor dyes (Thermo Fisher), DyLight dyes (Thermo Fisher), Cy dyes (GE Healthscience), IRDyes (Li-Cor Biosciences, Inc.), and HiLyte dyes (Anaspec, Inc.). In embodiments, a particular nucleotide type is associated with a particular label, such that identifying the label identifies the nucleotide with which it is associated. In embodiments, the label is luciferin that reacts with luciferase to produce a detectable signal in response to one or more bases being incorporated into an elongated complementary strand, such as in pyrosequencing. In embodiment, a nucleotide includes a label (such as a dye). In embodiments, the label is not associated with any particular nucleotide, but detection of the label identifies whether one or more nucleotides having a known identity were added during an extension step (such as in the case of pyrosequencing).

In embodiments, the detectable label is a fluorescent dye. In embodiments, the detectable label is a fluorescent dye capable of exchanging energy with another fluorescent dye (e.g., fluorescence resonance energy transfer (FRET) chromophores). Examples of detectable agents include imaging agents, including fluorescent and luminescent substances, including, but not limited to, a variety of organic or inorganic small molecules commonly referred to as “dyes,” “labels,” or “indicators.” Examples include fluorescein, rhodamine, acridine dyes, Alexa dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). In embodiments, the detectable moiety is a moiety of a derivative of one of the detectable moieties described immediately above, wherein the derivative differs from one of the detectable moieties immediately above by a modification resulting from the conjugation of the detectable moiety to a compound described herein.

As used herein, the term “polymerase” and “nucleic acid polymerase” are used in accordance with their plain ordinary meanings and refer to enzymes capable of synthesizing nucleic acid molecules from nucleotides (e.g., deoxyribonucleotides). Exemplary types of polymerases that may be used in the compositions and methods of the present disclosure include the nucleic acid polymerases such as DNA polymerase, DNA- or RNA-dependent RNA polymerase, and reverse transcriptase. In some cases, the DNA polymerase is 9° N polymerase or a variant thereof, E. Coli DNA polymerase I, Bacteriophage T4 DNA polymerase, Sequenase, Taq DNA polymerase, DNA polymerase from Bacillus stearothermophilus, Bst 2.0 DNA polymerase, 9° N polymerase (exo-)A485L/Y409V, Phi29 DNA Polymerase (φ29 DNA Polymerase), T7 DNA polymerase, DNA polymerase II, DNA polymerase III holoenzyme, DNA polymerase IV, DNA polymerase V, VentR DNA polymerase, Therminator™ II DNA Polymerase, Therminator™ III DNA Polymerase, or or Therminator™ IX DNA Polymerase. In embodiments, the polymerase is a protein polymerase. Typically, a DNA polymerase adds nucleotides to the 3′-end of a DNA strand, one nucleotide at a time. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol υ DNA polymerase, or a thermophilic nucleic acid polymerase (e.g. Therminator γ, 9° N polymerase (exo-), Therminator™ II, Therminator™ III, or Therminator™ IX). In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044). In embodiments, the polymerase is a reverse transcriptase such as HIV type M or O reverse transcriptase, avian myeloblastosis virus reverse transcriptase, or Moloney Murine Leukemia Virus (MMLV) reverse transcriptase, or telomerase. In embodiments, the polymerase is an enzyme described in US 2021/0139884. For example, a polymerase catalyzes the addition of a next correct nucleotide to the 3′-OH group of the primer via a phosphodiester bond, thereby chemically incorporating the nucleotide into the primer. Optionally, the polymerase used in the provided methods is a processive polymerase. Optionally, the polymerase used in the provided methods is a distributive polymerase.

As used herein, the term “thermophilic nucleic acid polymerase” refers to a family of DNA polymerases (e.g., 9° N™) and mutants thereof derived from the DNA polymerase originally isolated from the hyperthermophilic archaea, Thermococcus sp. 9 degrees N-7, found in hydrothermal vents at that latitude (East Pacific Rise) (Southworth M W, et al. PNAS. 1996; 93(11):5281-5285). A thermophilic nucleic acid polymerase is a member of the family B DNA polymerases. Site-directed mutagenesis of the 3′-5′ exo motif I (Asp-Ile-Glu or DIE) to AIA, AIE, EIE, EID or DIA yielded polymerase with no detectable 3′ exonuclease activity. Mutation to Asp-Ile-Asp (DID) resulted in reduction of 3′-5′ exonuclease specific activity to <1% of wild type, while maintaining other properties of the polymerase including its high strand displacement activity. The sequence AIA (D141A, E143A) was chosen for reducing exonuclease. Subsequent mutagenesis of key amino acids results in an increased ability of the enzyme to incorporate dideoxynucleotides, ribonucleotides and acyclonucleotides (e.g., Therminator II enzyme from New England Biolabs with D141A/E143A/Y409V/A485L mutations); 3′-amino-dNTPs, 3′-azido-dNTPs and other 3′-modified nucleotides (e.g., NEB Therminator III DNA Polymerase with D141A/E143A/L408S/Y409A/P410V mutations, NEB Therminator IX DNA polymerase), or 7-phosphate labeled nucleotides (e.g., Therminator γ: D141A/E143A/W355A/L408W/R460A/Q461S/K464E/D480V/R484W/A485L). Typically, these enzymes do not have 5′-3′ exonuclease activity. Additional information about thermophilic nucleic acid polymerases may be found in (Southworth M W, et al. PNAS. 1996; 93(11):5281-5285; Bergen K, et al. ChemBioChem. 2013; 14(9):1058-1062; Kumar S, et al. Scientific Reports. 2012; 2:684; Fuller C W, et al. 2016; 113(19):5233-5238; Guo J, et al. Proceedings of the National Academy of Sciences of the United States of America. 2008; 105(27):9145-9150), which are incorporated herein in their entirety for all purposes.

As used herein, the term “exonuclease activity” is used in accordance with its ordinary meaning in the art, and refers to the removal of a nucleotide from a nucleic acid by a DNA polymerase. For example, during polymerization, nucleotides are added to the 3′ end of the primer strand. Occasionally a DNA polymerase incorporates an incorrect nucleotide to the 3′-OH terminus of the primer strand, wherein the incorrect nucleotide cannot form a hydrogen bond to the corresponding base in the template strand. Such a nucleotide, added in error, is removed from the primer as a result of the 3′ to 5′ exonuclease activity of the DNA polymerase. In embodiments, exonuclease activity may be referred to as “proofreading.” When referring to 3′-5′ exonuclease activity, it is understood that the DNA polymerase facilitates a hydrolyzing reaction that breaks phosphodiester bonds at the 3′ end of a polynucleotide chain to excise the nucleotide. In embodiments, 3′-5′ exonuclease activity refers to the successive removal of nucleotides in single-stranded DNA in a 3′→5′ direction, releasing deoxyribonucleoside 5′-monophosphates one after another. Methods for quantifying exonuclease activity are known in the art, see for example Southworth et al, PNAS Vol 93, 8281-8285 (1996). In embodiments, 5′-3′ exonuclease activity refers to the successive removal of nucleotides in double-stranded DNA in a 5′→3′ direction. In embodiments, the 5′-3′ exonuclease is lambda exonuclease. For example, lambda exonuclease catalyzes the removal of 5′ mononucleotides from duplex DNA, with a preference for 5′ phosphorylated double-stranded DNA. In other embodiments, the 5′-3′ exonuclease is E. coli DNA Polymerase I.

The terms “DNA ligase” and “ligase” are used in accordance with their ordinary meaning in the art and refer to an enzyme capable catalyzing the formation of a phosphodiester bond between two nucleic acids. In embodiments, the DNA ligase covalently joins the phosphate backbone of a nucleic acid with a compatible nucleotide residue (e.g., a second blunt ended strand). In embodiments, the ligase is a ligation enzyme (e.g., CircLigase™ enzyme, Taq DNA Ligase, HiFi Taq DNA Ligase, T4 ligase, PBCV-1 DNA Ligase (also known as SplintR ligase) or Ampligase DNA Ligase). Non-limiting examples of ligases include DNA ligases such as DNA Ligase I, DNA Ligase II, DNA Ligase III, DNA Ligase IV, T4 DNA ligase, T7 DNA ligase, T3 DNA Ligase, E. coli DNA Ligase, PBCV-1 DNA Ligase (also known as SplintR ligase) or a Taq DNA Ligase. In embodiments, a ligase is provided in a buffer containing ATP and a divalent ion (e.g., Mn²⁺ or Mg²⁺). In embodiments, the ligase is provided in a buffer containing PEG, which is known to increase the ligation efficiency of nucleic acid molecules. As used herein, the term “exonuclease activity” is used in accordance with its ordinary meaning in the art, and refers to the removal of a nucleotide from a nucleic acid by a DNA polymerase. For example, during polymerization, nucleotides are added to the 3′ end of a primer or extension strand. Occasionally, a DNA polymerase incorporates an incorrect nucleotide to the 3′-OH terminus of the primer strand, wherein the incorrect nucleotide cannot form a hydrogen bond to the corresponding base in the template strand. Such a nucleotide, added in error, is removed from the primer or extension product as a result of the 3′ to 5′ exonuclease activity of the DNA polymerase. In embodiments, exonuclease activity may be referred to as “proofreading.” When referring to 3′-5′ exonuclease activity, it is understood that the DNA polymerase facilitates a hydrolyzing reaction that breaks phosphodiester bonds at either the 3′ end of a polynucleotide chain to excise the nucleotide. In embodiments, 3 3′-5′ exonuclease activity refers to the successive removal of nucleotides in single-stranded DNA in a 3′→5′ direction, releasing deoxyribonucleoside 5′-monophosphates one after another. Methods for quantifying exonuclease activity are known in the art, for example Southworth et al. PNAS Vol 93, 8281-8285 (1996).

As used herein, the term “incorporating” or “chemically incorporating,” when used in reference to a primer and cognate nucleotide, refers to the process of joining the cognate nucleotide to the primer or extension product thereof by formation of a phosphodiester bond.

As used herein, the term “selective” or “selectivity” or the like of a compound refers to the compound's ability to discriminate between molecular targets. When used in the context of sequencing, such as in “selectively sequencing,” this term refers to sequencing one or more target polynucleotides from an original starting population of polynucleotides, and not sequencing non-target polynucleotides from the starting population. Typically, selectively sequencing one or more target polynucleotides involves differentially manipulating the target polynucleotides based on known sequence. For example, target polynucleotides may be hybridized to a probe oligonucleotide that may be labeled (such as with a member of a binding pair) or bound to a surface. In embodiments, hybridizing a target polynucleotide to a probe oligonucleotide includes the step of displacing one strand of a double-stranded nucleic acid. Probe-hybridized target polynucleotides may then be separated from non-hybridized polynucleotides, such as by removing probe-bound polynucleotides from the starting population or by washing away polynucleotides that are not bound to a probe. The result is a selected subset of the starting population of polynucleotides, which is then subjected to sequencing, thereby selectively sequencing the one or more target polynucleotides.

As used herein, the terms “specific”, “specifically”, and “specificity”, are used in accordance with their ordinary meaning in the art, and in the context of a compound refer to the compound's ability to cause a particular action, such as binding, to a particular molecular target with minimal or no action to other proteins in the cell.

As used herein, the terms “bind” and “bound” are used in accordance with their plain and ordinary meanings and refer to an association between atoms or molecules. The association can be direct or indirect. For example, bound atoms or molecules may be directly bound to one another, e.g., by a covalent bond or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). As a further example, two molecules may be bound indirectly to one another by way of direct binding to one or more intermediate molecules, thereby forming a complex.

As used herein, the term “rolling circle amplification (RCA)” refers to a nucleic acid amplification reaction that amplifies a circular nucleic acid template (e.g., single-stranded DNA circles) via a rolling circle mechanism. Rolling circle amplification reaction is initiated by the hybridization of a primer to a circular, often single-stranded, nucleic acid template. The nucleic acid polymerase then extends the primer that is hybridized to the circular nucleic acid template by continuously progressing around the circular nucleic acid template to replicate the sequence of the nucleic acid template over and over again (rolling circle mechanism). The rolling circle amplification typically produces concatemers including tandem repeat units of the circular nucleic acid template sequence. The rolling circle amplification may be a linear RCA (LRCA), exhibiting linear amplification kinetics (e.g., RCA using a single specific primer), or may be an exponential RCA (ERCA) exhibiting exponential amplification kinetics. Rolling circle amplification may also be performed using multiple primers (multiply primed rolling circle amplification or MPRCA) leading to hyper-branched concatemers. For example, in a double-primed RCA, one primer may be complementary, as in the linear RCA, to the circular nucleic acid template, whereas the other may be complementary to the tandem repeat unit nucleic acid sequences of the RCA product. Consequently, the double-primed RCA may proceed as a chain reaction with exponential (geometric) amplification kinetics featuring a ramifying cascade of multiple-hybridization, primer-extension, and strand-displacement events involving both the primers. This often generates a discrete set of concatemeric, double-stranded nucleic acid amplification products. The rolling circle amplification may be performed in-vitro under isothermal conditions using a suitable nucleic acid polymerase such as Phi29 DNA polymerase. RCA may be performed by using any of the DNA polymerases that are known in the art (e.g., a Phi29 DNA polymerase, a Bst DNA polymerase, or SD polymerase).

As used herein, the term “extension” or “elongation” is used in accordance with its plain and ordinary meanings and refer to synthesis by a polymerase of a new polynucleotide strand complementary to a template strand by adding free nucleotides (e.g., dNTPs) from a reaction mixture that are complementary to the template in the 5′-to-3′ direction. Extension includes condensing the 5′-phosphate group of the dNTPs with the 3′-hydroxy group at the end of the nascent (elongating) DNA strand.

As used herein, the term “hybridization pad” or “hybridization sequence” refers to one or both of two regions on either end of an interposing oligonucleotide barcode that are capable of hybridizing to single-stranded template nucleic acids. In embodiments, hybridization sequences are a complement to the original target nucleic acid. In embodiments, each hybridization sequence is composed of about 3 to about 40 random nucleotides (e.g. NNNNN, wherein N represents A, T, C, G nucleotides). In embodiments, each hybridization sequence is composed of about 3 to about 5 random nucleotides. In embodiments, the first hybridization sequence includes about 3 to about 5 nucleotides (e.g., random nucleotides) and the second hybridization sequence includes about 3 to 25 nucleotides (e.g., random nucleotides). In embodiments, the first hybridization sequence includes about 5 to about 15 nucleotides (e.g., random nucleotides) and the second hybridization sequence includes about 5 to 15 nucleotides (e.g., random nucleotides). In embodiments, the first hybridization sequence includes about 10 to about 15 nucleotides (e.g., random nucleotides) and the second hybridization sequence includes about 10 to 15 nucleotides (e.g., random nucleotides). In embodiments, the hybridization sequence includes a targeted primer sequence, or a portion thereof. A “targeted primer sequence” refers to a nucleic acid sequence that is complementary to a known nucleic acid region (e.g., complementary to a universally conserved region, or complementary sequences to target specific genes or mutations that have relevancy to a particular cancer phenotype). The hybridization sequences may include sequences designed through computational software, e.g., Primer BLAST, LaserGene (DNAStar), Oligo (National Biosciences, Inc.), MacVector (Kodak/IBI) or the GCG suite of programs to optimize desired properties. In embodiments, the hybridization sequence includes a limited-diversity sequence. A “limited-diversity sequence” refers to a nucleic acid sequence that includes random nucleotide regions and fixed nucleotide regions (e.g., NNANN, ANNTN, TNCNA, etc., wherein N represents random nucleotides and A, T, C, G represent fixed nucleotides). In embodiments, each hybridization sequence is composed of 3 random nucleotides and 1 to 2 non-random nucleotides. In embodiments, each hybridization sequence is composed of 4 random nucleotides and 1 to 2 non-random nucleotides.

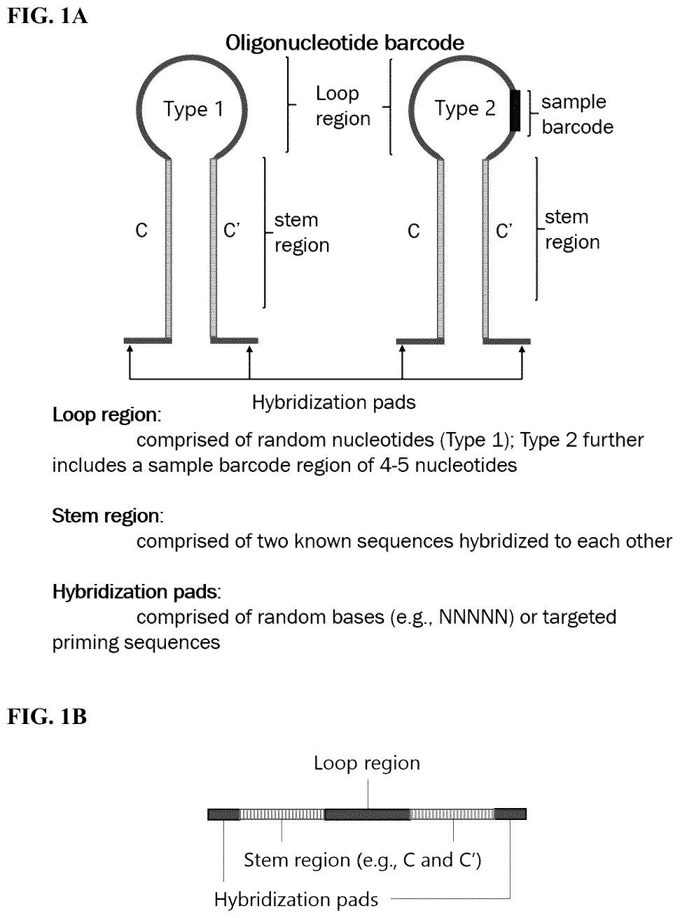

As used herein, the term “stem region” or “stem” refers to a region of an interposing oligonucleotide barcode that includes two known sequences capable of hybridizing to each other. In embodiments, the stem includes about 5 to about 10 nucleotides, and is stable (i.e., capable to remaining hybridized together) at approximately 37° C., and unhybridizes (i.e., denatures) at temperatures greater than 50° C. As the stem is of known or pre-determined sequence (i.e., non-random sequence), the stem sequences allow for location identification of interposing oligonucleotide barcodes. In embodiments, the stem region includes two regions of the same strand that are complementary separated by a loop region; see for example FIG. 1A.

As used herein, the term “loop region” or “loop” refers to a region of an interposing oligonucleotide barcode that is between sequences of the stem region, and remains single-stranded when sequences of the stem region are hybridized to one another. In embodiments, the loop includes about 10 to about 20 random nucleotides. In embodiments, the loop includes a modified nucleotide (e.g., a nucleotide linked to an affinity tag). In embodiments, the loop includes a biotinylated nucleotide (e.g., biotin-11-cytidine-5′-triphosphate). In embodiments, the loop region includes a barcode sequence. See, for example, FIG. 1A. In embodiments, the loop includes a limited-diversity sequence. For example, in embodiments, the loop includes a TT-[UMI]-TT sequence, such as TT-[NNNNNNNNNNNN]-TT (SEQ ID NO:1) sequence, wherein N represents random nucleotides and A, T, C, G represent fixed nucleotides).

As used herein, the term “barcode sequence” (which may be referred to as a “tag,” a “molecular barcode,” a “molecular identifier,” an “identifier sequence,” or a “unique molecular identifier”) refers to any material (e.g., a nucleotide sequence, a nucleic acid molecule feature) that is capable of distinguishing an individual molecule in a large heterogeneous population of molecules. Generally, a barcode sequence is unique in a pool of barcode sequences that differ from one another in sequence, or is uniquely associated with a particular sample polynucleotide in a pool of sample polynucleotides. In embodiments, the barcode sequence is a nucleotide sequence that forms a portion of a larger polynucleotide, such as an “interposing oligonucleotide barcode” (also referred to herein as an “interposing barcode” or an “oligonucleotide barcode”). In embodiments, every barcode sequence in a pool of interposing oligonucleotide barcodes is unique, such that sequencing reads including the barcode sequence can be identified as originating from a single sample polynucleotide molecule on the basis of the barcode sequence alone. In other embodiments, individual barcode sequences may be used more than once, but interposing oligonucleotide barcodes including the duplicate barcode sequences hybridize to different sample polynucleotides and/or in different arrangements of neighboring interposing oligonucleotide barcodes, such that sequence reads may still be uniquely distinguished as originating from a single sample polynucleotide molecule on the basis of a barcode sequence and adjacent sequence information (e.g., sample polynucleotide sequence, and/or one or more adjacent barcode sequences). In embodiments, barcode sequences are about or at least about 5, 6, 7, 8, 9, 10, 15, 20, 25, 30, 40, 50, 75 or more nucleotides in length. In embodiments, barcode sequences are shorter than 20, 15, 10, 9, 8, 7, 6, or 5 nucleotides in length. In embodiments, barcode sequences are about 10 to about 50 nucleotides in length, such as about 15 to about 40 or about 20 to about 30 nucleotides in length. In a pool of different barcode sequences, barcode sequences may have the same or different lengths. In general, barcode sequences are of sufficient length and include sequences that are sufficiently different to allow the identification of sequencing reads that originate from the same sample polynucleotide molecule. In embodiments, each barcode sequence in a plurality of barcode sequences differs from every other barcode sequence in the plurality by at least three nucleotide positions, such as at least 3, 4, 5, 6, 7, 8, 9, 10, or more nucleotide positions. In some embodiments, substantially degenerate barcode sequences may be known as random. In some embodiments, a barcode sequence may include a nucleic acid sequence from within a pool of known sequences. In some embodiments, the barcode sequences may be pre-defined.

As used herein, the term “random” in the context of a nucleic acid sequence or barcode sequence refers to a sequence where one or more nucleotides has an equal probability of being present. In embodiments, one or more nucleotides is selected at random from a set of two or more different nucleotides at one or more positions, with each of the different nucleotides selected at one or more positions represented in a pool of oligonucleotides including the random sequence. For example, a random sequence may be represented by a sequence composed of N's, where N can be any nucleotide (e.g., A, T, C, or G). For example, a four base random sequence may have the sequence NNNN, where the Ns can independently be any nucleotide (e.g., AATC). IBCs that contain a random sequence, collectively, have sequences composed of Ns within the hybridization sequences, stem region, or loop region. Further, the IBCs have barcode sequences that may contain random sequence. In embodiments, a pool of IBCs may be represented by a fully random sequence, with the caveat that certain sequences have been excluded (e.g., runs of three or more nucleotides of the same type, such as “AAA” or “GGG”). In embodiments, nucleotide positions that are allowed to vary (e.g., by two, three, or four nucleotides) may be separated by one or more fixed positions (e.g., as in “NGN”).

As used herein, the terms “denaturant” or plural “denaturants” are used in accordance with their plain and ordinary meanings and refer to an additive or condition that disrupts the base pairing between nucleotides within opposing strands of a double-stranded polynucleotide molecule. The term “denature” and its variants, when used in reference to any double-stranded polynucleotide molecule, or double-stranded polynucleotide sequence, includes any process whereby the base pairing between nucleotides within opposing strands of the double-stranded molecule, or double-stranded sequence, is disrupted. Typically, denaturation includes rendering at least some portion or region of two strands of the double-stranded polynucleotide molecule or sequence single-stranded or partially single-stranded. In some embodiments, denaturation includes separation of at least some portion or region of two strands of the double-stranded polynucleotide molecule or sequence from each other. Typically, the denatured region or portion is then capable of hybridizing to another polynucleotide molecule or sequence. Optionally, there can be “complete” or “total” denaturation of a double-stranded polynucleotide molecule or sequence. Complete denaturation conditions are, for example, conditions that would result in complete separation of a significant fraction (e.g., more than 10%, 20%, 30%, 40% or 50%) of a large plurality of strands from their extended and/or full-length complements. Typically, complete or total denaturation disrupts all of the base pairing between the nucleotides of the two strands with each other. Similarly, a nucleic acid sample is optionally considered fully denatured when more than 80% or 90% of individual molecules of the sample lack any double-strandedness (or lack any hybridization to a complementary strand).

Optionally, a nucleic acid sample can be considered to be partially denatured when a substantial fraction of individual nucleic acid molecules of the sample (e.g., above 20%, 30%, 50%, or 70%) are in a partially denatured state. Optionally less than a substantial amount of individual nucleic acid molecules in the sample are fully denatured, e.g., not more than 5%, 10%, 20%, 30% or 50% of the nucleic acid molecules in the sample. Under exemplary conditions at least 50% of the nucleic acid molecules of the sample are partly denatured, but less than 20% or 10% are fully denatured. In other situations, at least 30% of the nucleic acid molecules of the sample are partly denatured, but less than 10% or 5% are fully denatured. Similarly, a nucleic acid sample can be non-denatured when a minority of individual nucleic acid molecules in the sample are partially or completely denatured.

In an embodiment, partially denaturing conditions are achieved by maintaining the duplexes as a suitable temperature range. For example, the nucleic acid is maintained at temperature sufficiently elevated to achieve some heat-denaturation (e.g., above 45° C., 50° C., 55° C., 60° C., 65° C., or 70° C.) but not high enough to achieve complete heat-denaturation (e.g., below 95° C. or 90° C. or 85° C. or 80° C. or 75° C.). In an embodiment the nucleic acid is partially denatured using substantially isothermal conditions. Alternatively, chemical denaturation can be accomplished by contacting the double-stranded polynucleotide to be denatured with appropriate chemical denaturants, such as strong alkalis, strong acids, chaotropic agents, and the like and can include, for example, NaOH, urea, or guanidine-containing compounds. In some embodiments, partial or complete denaturation is achieved by exposure to chemical denaturants such as urea or formamide, with concentrations suitably adjusted, or using high or low pH (e.g., pH between 4-6 or 8-9). In embodiments, the denaturant is a buffered solution including betaine, dimethyl sulfoxide (DMSO), ethylene glycol, formamide, glycerol, guanidine thiocyanate, 4-methylmorpholine 4-oxide (NMO), or a mixture thereof. In embodiments, the first denaturant is a buffered solution including about 0% to about 50% dimethyl sulfoxide (DMSO); about 0% to about 50% ethylene glycol; about 0% to about 20% formamide; or about 0 to about 3M betaine, or a mixture thereof. In an embodiment herein, partial denaturation and/or amplification, including any one or more steps or methods described herein, can be achieved using a recombinase and/or single-stranded binding protein.

In some embodiments, complete or partial denaturation of a double-stranded polynucleotide sequence is accomplished by contacting the double-stranded polynucleotide sequence using appropriate denaturing agents. For example, the double-stranded polynucleotide can be subjected to heat-denaturation (also referred to interchangeably as thermal denaturation) by raising the temperature to a point where the desired level of denaturation is accomplished. In some embodiments, thermal denaturation of a double-stranded polynucleotide, includes adjusting the temperature to achieve complete separation of the two strands of the polynucleotide, such that 90% or greater of the strands are in single-stranded form across their entire length. A completely denatured double-stranded polynucleotide results in a separated first strand and a second strand, each of which is a single-stranded polynucleotide. In some embodiments, complete thermal denaturation of a polynucleotide molecule (or polynucleotide sequence) is accomplished by exposing the polynucleotide molecule (or sequence) to a temperature that is at least 5° C., 10° C., 15° C., 20° C., 25° C., 30° C., 50° C., or 100° C., above the calculated or predict melting temperature (Tm) of the polynucleotide molecule or sequence.

In some embodiments, complete or partial denaturation is accomplished by treating the double-stranded polynucleotide sequence to be denatured using a denaturant mixture including an SSB protein (e.g., T4 gp32 protein, T7 gene 2.5 SSB protein, or phi29 SSB protein, Thermococcus kodakarensis (KOD) SSB, Thermus thermophilus (TTH) SSB, Sulfolobus solfataricus (SSO) SSB, or Extreme Thermostable Single-Stranded DNA Binding Protein (ET-SSB)), a strand-displacing polymerase (e.g., Bst large fragment (Bst LF) polymerase, Bst 3.0 polymerase, Bst 2.0 polymerase, Bsu polymerase, SD polymerase, Vent exo-polymerase, Phi29 polymerase, or a mutant thereof), and one or more crowding agents (poly(ethylene glycol) (PEG), polyvinylpyrrolidone (PVP), bovine serum albumin (BSA), dextran, Ficoll (e.g., Ficoll 70 or Ficoll 400), glycerol, or a combination thereof). In embodiments, the crowding agent is poly(ethylene glycol) (e.g., PEG 200, PEG 600, PEG 800, PEG 2,050, PEG 4,600, PEG 6,000, PEG 8,000, PEG 10,000, PEG 20,000, or PEG 35,000), dextran sulfate, bovine pancreatic trypsin inhibitor (BPTI), ribonuclease A, lysozyme, β-lactoglobulin, hemoglobin, bovine serum albumin (BSA), or poly(sodium 4-styrene sulfonate) (PSS). In embodiments, the denaturant mixture including an SSB, a strand-displacing polymerase, and one or more crowding agents does not include a chemical denaturant (e.g., betaine, DMSO, ethylene glycol, formamide, guanidine thiocyanate, NMO, TMAC, or a mixture thereof).

As used herein, the terms “solid support” and “substrate” and “solid surface” refer to discrete solid or semi-solid surfaces to which a plurality of primers may be attached. A solid support may encompass any type of solid, porous, or hollow sphere, ball, cylinder, or other similar configuration composed of plastic, ceramic, metal, or polymeric material (e.g., hydrogel) onto which a nucleic acid may be immobilized (e.g., covalently or non-covalently). A solid support may include a discrete particle that may be spherical (e.g., microspheres) or have a non-spherical or irregular shape, such as cubic, cuboid, pyramidal, cylindrical, conical, oblong, or disc-shaped, and the like. Solid supports may be in the form of discrete particles, which alone does not imply or require any particular shape. The term “particle” means a small body made of a rigid or semi-rigid material. The body can have a shape characterized, for example, as a sphere, oval, microsphere, or other recognized particle shape whether having regular or irregular dimensions. As used herein, the term “discrete particles” refers to physically distinct particles having discernible boundaries. The term “particle” does not indicate any particular shape. The shapes and sizes of a collection of particles may be different or about the same (e.g., within a desired range of dimensions, or having a desired average or minimum dimension). A particle may be substantially spherical (e.g., microspheres) or have a non-spherical or irregular shape, such as cubic, cuboid, pyramidal, cylindrical, conical, oblong, or disc-shaped, and the like. In embodiments, the particle has the shape of a sphere, cylinder, spherocylinder, or ellipsoid. Discrete particles collected in a container and contacting one another will define a bulk volume containing the particles, and will typically leave some internal fraction of that bulk volume unoccupied by the particles, even when packed closely together. In embodiments, cores and/or core-shell particles are approximately spherical. As used herein the term “spherical” refers to structures which appear substantially or generally of spherical shape to the human eye, and does not require a sphere to a mathematical standard. In other words, “spherical” cores or particles are generally spheroidal in the sense of resembling or approximating to a sphere. In embodiments, the diameter of a spherical core or particle is substantially uniform, e.g., about the same at any point, but may contain imperfections, such as deviations of up to 1, 2, 3, 4, 5 or up to 10%. Because cores or particles may deviate from a perfect sphere, the term “diameter” refers to the longest dimension of a given core or particle. Likewise, polymer shells are not necessarily of perfect uniform thickness all around a given core. Thus, the term “thickness” in relation to a polymer structure (e.g., a shell polymer of a core-shell particle) refers to the average thickness of the polymer layer.

A solid support may further include a polymer or hydrogel on the surface to which the primers are attached (e.g., the splint primers are covalently attached to the polymer, wherein the polymer is in direct contact with the solid support). Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zeonor, silica or silica-based materials including silicon and modified silicon, carbon, metals, inorganic glasses, optical fiber bundles, photopatternable dry film resists, UV-cured adhesives and polymers. The solid supports for some embodiments have at least one surface located within a flow cell. The solid support, or regions thereof, can be substantially flat. The solid support can have surface features such as wells, pits, channels, ridges, raised regions, pegs, posts or the like. The term solid support is encompassing of a substrate (e.g., a flow cell) having a surface including a polymer coating covalently attached thereto. In embodiments, the solid support is a flow cell. The term “flow cell” as used herein refers to a chamber including a solid surface across which one or more fluid reagents can be flowed. Examples of flow cells and related fluidic systems and detection platforms that can be readily used in the methods of the present disclosure are described, for example, in Bentley et al., Nature 456:53-59 (2008). In certain embodiments a substrate includes a surface (e.g., a surface of a flow cell, a surface of a tube, a surface of a chip, surface of a particle), for example a metal surface (e.g., steel, gold, silver, aluminum, silicon and copper). In some embodiments a substrate (e.g., a substrate surface) is coated and/or includes functional groups and/or inert materials. In certain embodiments a substrate includes a bead, a chip, a capillary, a plate, a membrane, a wafer (e.g., silicon wafers), a comb, or a pin for example. In some embodiments a substrate includes a bead and/or a nanoparticle. A substrate can be made of a suitable material, non-limiting examples of which include a plastic or a suitable polymer (e.g., polycarbonate, poly(vinyl alcohol), poly(divinylbenzene), polystyrene, polyamide, polyester, polyvinylidene difluoride (PVDF), polyethylene, polyurethane, polypropylene, and the like), borosilicate, silica, nylon, Wang resin, Merrifield resin, metal (e.g., iron, a metal alloy, sepharose, agarose, polyacrylamide, dextran, cellulose and the like or combinations thereof. In some embodiments a substrate includes a magnetic material (e.g., iron, nickel, cobalt, platinum, aluminum, and the like). In certain embodiments a substrate includes a magnetic bead (e.g., DYNABEADS®, hematite, AMPure XP). Magnets can be used to purify and/or capture nucleic acids bound to certain substrates (e.g., substrates including a metal or magnetic material).

As used herein, the term “polymer” refers to macromolecules having one or more structurally unique repeating units. The repeating units are referred to as “monomers,” which are polymerized for the polymer. Typically, a polymer is formed by monomers linked in a chain-like structure. A polymer formed entirely from a single type of monomer is referred to as a “homopolymer.” A polymer formed from two or more unique repeating structural units may be referred to as a “copolymer.” A polymer may be linear or branched, and may be random, block, polymer brush, hyperbranched polymer, bottlebrush polymer, dendritic polymer, or polymer micelles. The term “polymer” includes homopolymers, copolymers, tripolymers, tetra polymers and other polymeric molecules made from monomeric subunits. Copolymers include alternating copolymers, periodic copolymers, statistical copolymers, random copolymers, block copolymers, linear copolymers and branched copolymers. The term “polymerizable monomer” is used in accordance with its meaning in the art of polymer chemistry and refers to a compound that may covalently bind chemically to other monomer molecules (such as other polymerizable monomers that are the same or different) to form a polymer. Polymers can be hydrophilic, hydrophobic, or amphiphilic, as known in the art. Thus, “hydrophilic polymers” are substantially miscible with water and include, but are not limited to, polyethylene glycol and the like. “Hydrophobic polymers” are substantially immiscible with water and include, but are not limited to, polyethylene, polypropylene, polybutadiene, polystyrene, polymers disclosed herein, and the like. “Amphiphilic polymers” have both hydrophilic and hydrophobic properties and are typically copolymers having hydrophilic segment(s) and hydrophobic segment(s). Polymers include homopolymers, random copolymers, and block copolymers, as known in the art. The term “homopolymer” refers, in the usual and customary sense, to a polymer having a single monomeric unit. The term “copolymer” refers to a polymer derived from two or more monomeric species. The term “random copolymer” refers to a polymer derived from two or more monomeric species with no preferred ordering of the monomeric species. The term “block copolymer” refers to polymers having two or homopolymer subunits linked by covalent bond. Thus, the term “hydrophobic homopolymer” refers to a homopolymer which is hydrophobic. The term “hydrophobic block copolymer” refers to two or more homopolymer subunits linked by covalent bonds and which is hydrophobic.

As used herein, the term “hydrogel” refers to a three-dimensional polymeric structure that is substantially insoluble in water, but which is capable of absorbing and retaining large quantities of water to form a substantially stable, often soft and pliable, structure. In embodiments, water can penetrate in between polymer chains of a polymer network, subsequently causing swelling and the formation of a hydrogel. In embodiments, hydrogels are super-absorbent (e.g., containing more than about 90% water) and can be included of natural or synthetic polymers.

The term “surface” is intended to mean an external part or external layer of a substrate. The surface can be in contact with another material such as a gas, liquid, gel, polymer, organic polymer, second surface of a similar or different material, metal, or coating. The surface, or regions thereof, can be substantially flat. The substrate and/or the surface can have surface features such as wells, pits, channels, ridges, raised regions, pegs, posts or the like.

As used herein, the terms “sequencing”, “sequence determination”, “determining a nucleotide sequence”, and the like include determination of a partial or complete sequence information, including the identification, ordering, or locations of the nucleotides that include the polynucleotide being sequenced, and inclusive of the physical processes for generating such sequence information. That is, the term includes sequence comparisons, consensus sequence determination, contig assembly, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide. In some embodiments, a sequencing process described herein includes contacting a template and an annealed primer with a suitable polymerase under conditions suitable for polymerase extension and/or sequencing. The sequencing methods are preferably carried out with the target polynucleotide arrayed on a solid substrate. Multiple target polynucleotides can be immobilized on the solid support through linker molecules, or can be attached to particles, e.g., microspheres, which can also be attached to a solid substrate. In embodiments, the solid substrate is in the form of a chip, a bead, a well, a capillary tube, a slide, a wafer, a filter, a fiber, a porous media, or a column. In embodiments, the solid substrate is gold, quartz, silica, plastic, glass, diamond, silver, metal, or polypropylene. In embodiments, the solid substrate is porous.

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

Provided herein are methods and compositions for analyzing a sample (e.g., sequencing nucleic acids within a sample). A sample (e.g., a sample including nucleic acid) can be obtained from a suitable subject. A sample can be isolated or obtained directly from a subject or part thereof. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. A fluid or tissue sample from which nucleic acid is extracted may be acellular (e.g., cell-free). Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may include cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid).

In some embodiments, a sample includes nucleic acid, or fragments thereof. A sample can include nucleic acids obtained from one or more subjects. In some embodiments a sample includes nucleic acid obtained from a single subject. In some embodiments, a sample includes a mixture of nucleic acids. A mixture of nucleic acids can include two or more nucleic acid species having different nucleotide sequences, different fragment lengths, different origins (e.g., genomic origins, cell or tissue origins, subject origins, the like or combinations thereof), or combinations thereof. A sample may include synthetic nucleic acid.

A subject can be any living or non-living organism, including but not limited to a human, non-human animal, plant, bacterium, fungus, virus or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a human subject. A subject can be a patient (e.g., a human patient). In some embodiments a subject is suspected of having a genetic variation or a disease or condition associated with a genetic variation.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., packaging, buffers, written instructions for performing a method, etc.) from one location to another. For example, kits include one or more enclosures (e.g., boxes) containing the relevant reaction reagents and/or supporting materials. As used herein, the term “fragmented kit” refers to a delivery system including two or more separate containers that each contain a subportion of the total kit components. The containers may be delivered to the intended recipient together or separately. For example, a first container may contain an enzyme for use in an assay, while a second container contains oligonucleotides. In contrast, a “combined kit” refers to a delivery system containing all of the components of a reaction assay in a single container (e.g., in a single box housing each of the desired components). The term “kit” includes both fragmented and combined kits. In embodiments, the kit includes vessels containing one or more enzymes, primers, adaptors, or other reagents as described herein. Vessels may include any structure capable of supporting or containing a liquid or solid material and may include, tubes, vials, jars, containers, tips, etc. In embodiments, a wall of a vessel may permit the transmission of light through the wall. In embodiments, the vessel may be optically clear. The kit may include the enzyme and/or nucleotides in a buffer. In embodiments, the buffer includes an acetate buffer, 3-(N-morpholino)propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(Cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer.

The term “primer” as used herein, is defined to be one or more nucleic acid fragments that specifically hybridize to a nucleic acid template. A primer can be of any length depending on the particular technique it will be used for. For example, PCR primers are generally between 10 and 40 nucleotides in length. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. The length and complexity of the nucleic acid fixed onto the nucleic acid template is not critical to the invention. One of skill can adjust these factors to provide optimum hybridization and signal production for a given hybridization procedure, and to provide the required resolution among different genes or genomic locations. The primer permits the addition of a nucleotide residue thereto, or oligonucleotide or polynucleotide synthesis therefrom, under suitable conditions well-known in the art. In an embodiment the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. A primer (a primer sequence) is a short, usually chemically synthesized oligonucleotide, of appropriate length, for example about 18-24 bases, sufficient to hybridize to a target nucleic acid (e.g. a single stranded nucleic acid) and permit the addition of a nucleotide residue thereto, or oligonucleotide or polynucleotide synthesis therefrom, under suitable conditions well-known in the art. In an embodiment the primer is a DNA primer, i.e. a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In embodiments the primer is an RNA primer. In embodiments, the primer is an amplification primer (e.g., a primer optimized for PCR amplification which can anneal with the ssDNA and serve as a binding site for a DNA polymerase). The melting temperature (Tm) of a primer can be modified (e.g., increased) to a desired Tm using a suitable method, for example by changing (e.g., increasing) GC content, changing (e.g., increasing) length and/or by the inclusion of modified nucleotides, nucleotide analogues and/or modified nucleotides bonds, non-limiting examples of which include locked nucleic acids (LNAs, e.g., bicyclic nucleic acids), bridged nucleic acids (BNAs, e.g., constrained nucleic acids), C5-modified pyrimidine bases (for example, 5-methyl-dC, propynyl pyrimidines, among others) and alternate backbone chemistries, for example peptide nucleic acids (PNAs), morpholinos, the like or combinations thereof. In embodiments, the primers include nucleotide analogues to increase binding stability (e.g., Locked Nucleic Acid bases (LNAs), 2′ fluoronucleotides, or PNAs). For example, a primer that includes synthetic analogue bases such as LNAs (e.g., LNAs as described in US 2003/0092905; U.S. Pat. No. 7,084,125, which are incorporated herein by reference for all purposes) may increase the Tm. The Tm can be increased by using intercalators or additives such as Ethidium bromide or SYBR Green I. In embodiments, the primer includes a plurality of LNAs (e.g., 2, 3, 4, 5, 6, 7, 8, 9, or 10 LNAs). In embodiments, the primer includes 2-6 LNAs. The ribose moiety of an LNA nucleotide is modified from a typical ribose ring structure by a methylene bridge that connects the 2′ oxygen atom and the 4′ carbon atom, and which locks the ribose in the 3′endo conformation. Such LNAs can include any natural purine or pyrimidine base or non-natural bases (e.g., inosine, chemically modified bases, etc.).

A “blocking element” refers to an agent (e.g., polynucleotide, protein, nucleotide) that reduces and/or inhibits nucleotide incorporation (i.e., extension of a primer) relative to the absence of the blocking element. In embodiments, the blocking element is a non-extendable oligomer (e.g., a 3′-blocked oligo). A blocking element on a nucleotide can be reversible, whereby the blocking moiety can be removed or modified to allow the 3′ hydroxyl to form a covalent bond with the 5′ phosphate of another nucleotide. For example, a reversible terminator may refer to a blocking moiety located, for example, at the 3′ position of the nucleotide and may be a chemically cleavable moiety such as an allyl group, an azidomethyl group or a methoxymethyl group. In embodiments the blocking moiety is not reversible (e.g., the blocking element including a blocking moiety irreversibly prevents extension). In embodiments, the blocking element includes an oligo having a 3′ dideoxynucleotide or similar modification to prevent extension by a polymerase and is used in conjunction with a non-strand displacing polymerase. In another example implementation, the blocking element includes one or more modified nucleotides including a cleavable linker (e.g., linked to the 5′, 3′, or the nucleobase) containing PEG, thereby blocking the extension. In another example implementation, the blocking element includes one or more modified nucleotides linked to biotin, to which a protein (e.g., streptavidin) can be bound, thereby blocking polymerase extension. In another example implementation, the blocking element includes a modified nucleotide, such as iso dGTP or iso dCTP, which are complementary to each other. In a reaction of polymerization lacking the appropriate complementary modified nucleotides, the extension of a primer is halted. In another example implementation, the blocking element includes one or more sequences which is recognized and bound by one or more single-stranded DNA-binding proteins, thereby blocking polymerase extension at the bound site. In another example implementation, the blocking element includes one or more sequences which are recognized and bound by one or more short RNA or PNA oligos, thereby blocking the extension by a DNA polymerase that cannot strand displace RNA or PNA. In embodiments, the blocking element includes locked nucleic acids (LNAs), Bis-locked nucleic acids (bisLNAs), twisted intercalating nucleic acids (TINAs), bridged nucleic acids (BNAs), 2′-O-methyl RNA:DNA chimeric nucleic acids, minor groove binder (MGB) nucleic acids, morpholino nucleic acids, C5-modified pyrimidine nucleic acids, peptide nucleic acids (PNAs), phosphorothioate nucleic acids, or combinations thereof. In embodiments, the blocking element includes phosphorothioate nucleic acids. In embodiments, the blocking element includes one or more locked nucleic acids (LNAs), 2-amino-deoxyadenosine (2-amino-dA), trimethoxystilbene-functionalized oligonucleotides (TFOs), Pyrene-functionalized oligonucleotides (PFOs), peptide nucleic acids (PNAs), or aminoethyl-phenoxazine-dC (AP-dC) nucleic acids. In embodiments, the blocking element includes 10 to 15 locked nucleic acids (LNAs). In embodiments, the blocking element includes one or more phosphorothioates at the 5′ end. In embodiments, the blocking element includes one or more LNAs at the 5′ end. In embodiments, the blocking element includes two or more consecutive LNAs at the 3′ end. In embodiments, the blocking element includes two or more consecutive LNAs at the 5′ end. In embodiments, the blocking element includes a plurality (e.g., 2 to 10) of synthetic nucleotides (e.g., LNAs) and a plurality (e.g., 2 to 10) canonical or native nucleotides (e.g., dNTPs). In embodiments, the blocking element includes one or more (e.g., 2 to 5) deoxyuracil nucleobases (dU). In embodiments, the one or more dU nucleobases are at or near the 3′ end of the blocking element (e.g., within 5 nucleotides of the 3′ end). In embodiments, the one or more dU nucleobases are distributed through the blocking element. In embodiments, the blocking element includes from 5′ to 3′ a plurality (e.g., 2 to 5) of phosphorothioate nucleic acids, followed by a plurality of synthetic nucleotides (e.g., LNAs), and a plurality (e.g., 2 to 10) of canonical bases. In embodiments, the blocking element is about 10 to 100 nucleotides in length. In embodiments, the blocking element is about 15 to about 40 nucleotides in length. In embodiments, the calculated or predicted melting temperature (Tm) of the blocking element is about 70° C. to about 95° C. In embodiments, the calculated or predicted melting temperature (Tm) of the blocking element is about 75° C. to about 85° C. In embodiments, the calculated or predicted melting temperature (Tm) of the blocking element is 75° C. to 85° C.

As used herein, the terms “hybridization sequence” or “hybridization pad” refers to one or both of two regions (e.g., “first hybridization sequence” and “second hybridization sequence”) on either end of an interposing oligonucleotide probe that are capable of hybridizing to single-stranded template nucleic acids. In embodiments, hybridization sequences are a complement to the original target nucleic acid. In embodiments, each hybridization sequence is composed of about 3 to about 40 nucleotides. In embodiments, each hybridization sequence is composed of about 3 to about 5 nucleotides. In embodiments, the first hybridization sequence includes about 3 to about 5 nucleotides and the second hybridization sequence includes about 3 to 25 nucleotides. In embodiments, the first hybridization sequence includes about 5 to about 15 nucleotides and the second hybridization sequence includes about 5 to 15 nucleotides. In embodiments, the first hybridization sequence includes about 10 to about 15 nucleotides and the second hybridization sequence includes about 10 to 15 nucleotides. In embodiments, the hybridization sequence includes a targeted primer sequence, or a portion thereof. A “targeted primer sequence” refers to a nucleic acid sequence that is complementary to a known nucleic acid region (e.g., complementary to a universally conserved region, or complementary sequences to target specific genes or mutations that have relevancy to a particular cancer phenotype). The hybridization sequences may include sequences designed through computational software, e.g., Primer BLAST, LaserGene (DNAStar), Oligo (National Biosciences, Inc.), MacVector (Kodak/IBI) or the GCG suite of programs to optimize desired properties. In embodiments, the hybridization sequence includes a limited-diversity sequence. A “limited-diversity sequence” refers to a nucleic acid sequence that includes random nucleotide regions and fixed nucleotide regions (e.g., NNANN, ANNTN, TNCNA, etc., wherein N represents random nucleotides and A, T, C, G represent fixed nucleotides). In embodiments, each hybridization sequence is composed of 3 random nucleotides and 1 to 2 non-random nucleotides. In embodiments, each hybridization sequence is composed of 4 random nucleotides and 1 to 2 non-random nucleotides.

As used herein, the term “sequencing reaction mixture” is used in accordance with its plain and ordinary meaning and refers to an aqueous mixture that contains the reagents necessary to allow a nucleotide or nucleotide analogue to be added to a DNA strand by a DNA polymerase. In embodiments, the sequencing reaction mixture includes a buffer. In embodiments, the buffer includes an acetate buffer, 3-(N-morpholino) propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(Cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), or salts (e.g., ammonium sulfate, magnesium chloride, sodium chloride, or potassium chloride).

As used herein, the term “sequencing read” is used in accordance with its plain and ordinary meaning and refers to an inferred sequence of base pairs (or base pair probabilities) corresponding to all or part of a single DNA fragment. Sequencing technologies vary in the length of reads produced. Reads of length 20-40 base pairs (bp) are referred to as ultra-short. Typical sequencers produce read lengths in the range of 100-500 bp. A sequencing read may include 10, 20, 30, 40, 50, 60, 70, 80, 90, 100, 150, 200, 250, or more nucleotide bases. Read length is a factor which can affect the results of biological studies. For example, longer read lengths improve the resolution of de novo genome assembly and detection of structural variants. In embodiments, a sequencing read includes reading a barcode and a template nucleotide sequence. In embodiments, a sequencing read includes reading a template nucleotide sequence. In embodiments, a sequencing read includes reading a barcode and not a template nucleotide sequence. In embodiments, a sequencing read includes a computationally derived string corresponding to the detected label. The sequence reads are optionally stored in an appropriate data structure for further evaluation. In embodiments, a first sequencing reaction can generate a first sequencing read. The first sequencing read can provide the sequence of a first region of the polynucleotide fragment. In embodiments, a second sequencing primer can initiate sequencing at a second location on the nucleic acid template. The second location can be distinct from the first location. In some cases, a 3′ terminal nucleotide of the second primer can hybridize to a location that is more than 5 nucleotides away from a binding site of a 3′ terminal nucleotide of the first primer. The second sequencing reaction can generate a second sequencing read. The second sequencing read can provide the sequence of a second region of the nucleic acid template which is distinct from the first region of the nucleic acid template. In some embodiments, the nucleic acid template is optionally subjected to one or more additional rounds of sequencing using additional sequencing primers, thereby generating additional sequencing reads. In embodiments, a sequencing read is about 25 nucleotide bases. In embodiments, a sequencing read is about 35 nucleotide bases. In embodiments, a sequencing read is about 45 nucleotide bases. In embodiments, a sequencing read is about 55 nucleotide bases. In embodiments, a sequencing read is about 65 nucleotide bases. In embodiments, a sequencing read is about 75 nucleotide bases. In embodiments, a sequencing read is about 85 nucleotide bases. In embodiments, a sequencing read is a string of characters representing the sequence of nucleotides. In embodiments, the length of a sequencing read corresponds to the length of the target sequence. In embodiments, the length of a sequencing read corresponds to the number of sequencing cycles. A sequencing read may be subjected to initial processing (often termed “pre-processing”) prior to annotation. Pre-processing includes filtering out low-quality sequences, sequence trimming to remove continuous low-quality nucleotides, merging paired-end sequences, or identifying and filtering out PCR repeats using known techniques in the art. The sequenced reads may then be assembled and aligned using bioinformatic algorithms known in the art. A sequencing read may be aligned to a reference sequence. In embodiments, a sequencing read includes a sequence corresponding to an interposing oligonucleotide probe sequence (e.g., a sequencing primer binding sequence of the interposing oligonucleotide probe). In embodiments, a sequencing read includes a computationally derived string corresponding to the detected complementary nucleotide (e.g., a labeled nucleotide). The sequence reads are optionally stored in an appropriate data structure for further evaluation. In embodiments, a first sequencing reaction can generate a first sequencing read. The first sequencing read can provide the sequence of a first region of the polynucleotide fragment. In embodiments, a second sequencing primer can initiate sequencing at a second location on the nucleic acid template. The second location can be distinct from the first location. In some cases, a 3′ terminal nucleotide of the second primer can hybridize to a location that is more than 5 nucleotides away from a binding site of a 3′ terminal nucleotide of the first primer. The second sequencing reaction can generate a second sequencing read. The second sequencing read can provide the sequence of a second region of the nucleic acid template which is distinct from the first region of the nucleic acid template. In some embodiments, the nucleic acid template is optionally subjected to one or more additional rounds of sequencing using additional sequencing primers, thereby generating additional sequencing reads.

The term “multiplexing” as used herein refers to an analytical method in which the presence and/or amount of multiple targets, e.g., multiple nucleic acid target sequences, can be assayed simultaneously by using the methods and devices as described herein, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

As used herein, the terms “cluster” and “colony” are used interchangeably to refer to a discrete site on a solid support that includes a plurality of immobilized polynucleotides and a plurality of immobilized complementary polynucleotides. The term “clustered array” refers to an array formed from such clusters or colonies. In this context the term “array” is not to be understood as requiring an ordered arrangement of clusters. The term “array” is used in accordance with its ordinary meaning in the art, and refers to a population of different molecules that are attached to one or more solid-phase substrates such that the different molecules can be differentiated from each other according to their relative location. An array can include different molecules that are each located at different addressable features on a solid-phase substrate. The molecules of the array can be nucleic acid primers, nucleic acid probes, nucleic acid templates or nucleic acid enzymes such as polymerases or ligases. Arrays useful in the invention can have densities that ranges from about 2 different features to many millions, billions or higher. The density of an array can be from 2 to as many as a billion or more different features per square cm. For example an array can have at least about 100 features/cm², at least about 1,000 features/cm², at least about 10,000 features/cm², at least about 100,000 features/cm², at least about 10,000,000 features/cm², at least about 100,000,000 features/cm², at least about 1,000,000,000 features/cm², at least about 2,000,000,000 features/cm² or higher. In embodiments, the arrays have features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

As used herein, the term “sequencing cycle” is used in accordance with its plain and ordinary meaning and refers to incorporating one or more nucleotides (e.g., nucleotide analogues) to the 3′ end of a polynucleotide with a polymerase, and detecting one or more labels that identify the one or more nucleotides incorporated. The sequencing may be accomplished by, for example, sequencing by synthesis, pyrosequencing, and the like. In embodiments, a sequencing cycle includes extending a complementary polynucleotide by incorporating a first nucleotide using a polymerase, wherein the polynucleotide is hybridized to a template nucleic acid, detecting the first nucleotide, and identifying the first nucleotide. An “extension strand” is formed as the one or more nucleotides are incorporated into a complementary polynucleotide hybridized to a template nucleic acid. The extension strand is complementary to the template nucleic acid. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove labels from each incorporated base. Reagents, enzymes and other substances can be removed between steps by washing. Cycles may include repeating these steps, and the sequence of each cluster is read over the multiple repetitions.

As used herein the term “determine” can be used to refer to the act of ascertaining, establishing or estimating. A determination can be probabilistic. For example, a determination can have an apparent likelihood of at least 50%, 75%, 90%, 95%, 98%, 99%, 99.9% or higher. In some cases, a determination can have an apparent likelihood of 100%. An exemplary determination is a maximum likelihood analysis or report. As used herein, the term “identify,” when used in reference to a thing, can be used to refer to recognition of the thing, distinction of the thing from at least one other thing or categorization of the thing with at least one other thing. The recognition, distinction or categorization can be probabilistic. For example, a thing can be identified with an apparent likelihood of at least 50%, 75%, 90%, 95%, 98%, 99%, 99.9% or higher. A thing can be identified based on a result of a maximum likelihood analysis. In some cases, a thing can be identified with an apparent likelihood of 100%.

A “gene” refers to a polynucleotide that is capable of conferring biological function after being transcribed and/or translated.

The methods and kits of the present disclosure may be applied, mutatis mutandis, to the sequencing of RNA, or to determining the identity of a ribonucleotide.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., packaging, buffers, written instructions for performing a method, etc.) from one location to another. For example, kits include one or more enclosures (e.g., boxes) containing the relevant reaction reagents and/or supporting materials. As used herein, the term “fragmented kit” refers to a delivery system including two or more separate containers that each contain a subportion of the total kit components. The containers may be delivered to the intended recipient together or separately. For example, a first container may contain an enzyme for use in an assay, while a second container contains oligonucleotides. In contrast, a “combined kit” refers to a delivery system containing all of the components of a reaction assay in a single container (e.g., in a single box housing each of the desired components). The term “kit” includes both fragmented and combined kits.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### II. Compositions & Kits

In an aspect is provided bridged polynucleotide including a complement of a first independent single-stranded polynucleotide sequence, a bridging oligonucleotide sequence, a complement of a second independent single-stranded polynucleotide sequence, and a plurality of interposing oligonucleotide probes.

In an aspect is provided a bridged polynucleotide including a complement of a first independent single-stranded polynucleotide, a bridging oligonucleotide, a complement of a second independent single-stranded polynucleotide, and a plurality of interposing oligonucleotide probes.

In an aspect is provided a bridged polynucleotide including a complement of a first independent single-stranded polynucleotide sequence, a bridging oligonucleotide sequence, a complement of a second independent single-stranded polynucleotide sequence, and a plurality of interposing oligonucleotide barcode adapters.

In embodiments, the bridging oligonucleotide (e.g., the bridging oligonucleotide sequence) is present between the first and second complement sequences. In embodiments, the bridging oligonucleotide sequence includes a linker sequence. In embodiments, the bridging oligonucleotide sequence includes a barcode sequence. In embodiments, the bridging oligonucleotide sequence includes more than one barcode sequence. In embodiments, the bridging oligonucleotide sequence includes two barcode sequences. In embodiments, one or more barcode sequences are known sequences. In embodiments, one or more barcode sequences are random sequences. In embodiments, the one or more barcode sequences include a mixture of known and random sequences.

As used herein, the terms “bridge oligonucleotide,” or “bridging oligonucleotide” refer to an oligonucleotide with a first region of complementarity to a first independent polynucleotide at the 5′ end (e.g., the upstream end) of the bridge oligonucleotide, and a second region of complementarity to a second independent polynucleotide at the 3′ end (e.g., the downstream end) of the bridge oligonucleotide. In embodiments, the first independent polynucleotide and the second independent polynucleotide are different or substantially different. In embodiments, the first independent polynucleotide and the second independent polynucleotide are the same or substantially the same. In embodiments, the bridge oligonucleotide includes, from 5′ to 3′: i. a first hybridization sequence complementary to a 3′ terminal sequence of the first independent polynucleotide; ii. a linker sequence; and iii. a second hybridization sequence complementary to a 5′ terminal sequence of the second independent polynucleotide.

As used herein, the terms “overlap oligonucleotides” or “overlapping oligonucleotides” refer to at least two oligonucleotides, wherein the 3′ end (e.g., the downstream end) of the first overlap oligonucleotide includes a first region of complementarity to a 3′ sequence of a first independent polynucleotide, and wherein the 3′ end of the second overlap oligonucleotide includes a second region of complementarity to a 3′ sequence of a second independent polynucleotide, wherein the 5′ ends (e.g., the upstream ends) of the first and second overlap oligonucleotides are complementary to each other. In embodiments, a first overlap oligonucleotide includes, from 3′ to 5′, a first hybridization sequence complementary to a first independent polynucleotide and a sequence complementary to a second overlap oligonucleotide. In embodiments, a second overlap oligonucleotide includes, from 3′ to 5′, a second hybridization sequence complementary to a second independent polynucleotide and a sequence complementary to the first overlap oligonucleotide. In embodiments, the first hybridization sequence is complementary to a 3′ sequence of the first independent polynucleotide. In embodiments, the second hybridization sequence is complementary to a 3′ sequence of the second independent polynucleotide.

In embodiments, the 5′ end of the bridge oligonucleotide includes about 5 to about 50 nucleotides complementary to the 3′ end of the first independent polynucleotide. In embodiments, the 5′ end of the bridge oligonucleotide includes about 15 to about 40 nucleotides complementary to the 3′ end of the first independent polynucleotide. In embodiments, the 5′ end of the bridge oligonucleotide includes about 5, 10, 15, 20, 25, 30, 35, 40, 45, 50, or more nucleotides complementary to the 3′ end of the first independent polynucleotide.

In embodiments, the 3′ end of the bridge oligonucleotide includes about 5 to about 50 nucleotides complementary to the 5′ end of the first independent polynucleotide. In embodiments, the 3′ end of the bridge oligonucleotide includes about 15 to about 40 nucleotides complementary to the 5′ end of the first independent polynucleotide. In embodiments, the 3′ end of the bridge oligonucleotide includes about 5, 10, 15, 20, 25, 30, 35, 40, 45, 50, or more nucleotides complementary to the 5′ end of the first independent polynucleotide.

In embodiments, the 5′ end of the first overlap oligonucleotide includes about 5 to about 50 nucleotides complementary to the 5′ end of the second overlap oligonucleotide. In embodiments, the 5′ end of the first overlap oligonucleotide includes about 15 to about 40 nucleotides complementary to the 5′ end of the second overlap oligonucleotide. In embodiments, the 5′ end of the first overlap oligonucleotide includes about 5, 10, 15, 20, 25, 30, 35, 40, 45, 50, or more nucleotides complementary to the 5′ end of the second overlap oligonucleotide.

In embodiments, the 3′ end of the first overlap oligonucleotide includes about 5 to about 50 nucleotides complementary to the 3′ end of the first independent polynucleotide. In embodiments, the 3′ end of the first overlap oligonucleotide includes about 15 to about 40 nucleotides complementary to the 3′ end of the first independent polynucleotide. In embodiments, the 3′ end of the first overlap oligonucleotide includes about 5, 10, 15, 20, 25, 30, 35, 40, 45, 50, or more nucleotides complementary to the 3′ end of the first independent polynucleotide.

In embodiments, the 3′ end of the second overlap oligonucleotide includes about 5 to about 50 nucleotides complementary to the 3′ end of the second independent polynucleotide. In embodiments, the 3′ end of the second overlap oligonucleotide includes about 15 to about 40 nucleotides complementary to the 3′ end of the second independent polynucleotide. In embodiments, the 3′ end of the second overlap oligonucleotide includes about 5, 10, 15, 20, 25, 30, 35, 40, 45, 50, or more nucleotides complementary to the 3′ end of the second independent polynucleotide.

In embodiments, the bridge oligonucleotide is about 20 to about 250 nucleotides in length. In embodiments, the bridge oligonucleotide is about 20 to 200 nucleotides, 30 to 175 nucleotides, 40 to 150 nucleotides, 50 to 125 nucleotides, or 75 to 100 nucleotides in length. In embodiments, the bridge oligonucleotide is about 20, 30, 40, 50, 75, 100, 125, 150, 175, 200, 225, 250, or more nucleotides in length.

In embodiments, the overlap oligonucleotide is about 20 to about 250 nucleotides in length. In embodiments, the overlap oligonucleotide is about 20 to 200 nucleotides, 30 to 175 nucleotides, 40 to 150 nucleotides, 50 to 125 nucleotides, or 75 to 100 nucleotides in length. In embodiments, the overlap oligonucleotide is about 20, 30, 40, 50, 75, 100, 125, 150, 175, 200, 225, 250, or more nucleotides in length.

In embodiments, the linker sequence includes about 5 to about 50 nucleotides. In embodiments, the linker sequence includes about 10 to about 50 nucleotides. In embodiments, the complement of the linker sequence includes about 15 to about 50 nucleotides. In embodiments, the linker sequence includes about 20 to about 50 nucleotides. In embodiments, the linker sequence includes about 10 to about 100 nucleotides. In embodiments, the linker sequence includes about 20 to about 100 nucleotides. In embodiments, the linker sequence includes about 30 to about 100 nucleotides. In embodiments, the linker sequence includes about 40 to about 100 nucleotides.

In embodiments, the complement of the first independent single-stranded polynucleotide includes about 5 to about 50 nucleotides. In embodiments, the complement of the first independent single-stranded polynucleotide includes about 10 to about 50 nucleotides. In embodiments, the complement of the first independent single-stranded polynucleotide includes about 15 to about 50 nucleotides. In embodiments, the complement of the first independent single-stranded polynucleotide includes about 20 to about 50 nucleotides. In embodiments, the complement of the first independent single-stranded polynucleotide includes about 10 to about 100 nucleotides. In embodiments, the complement of the first independent single-stranded polynucleotide includes about 20 to about 100 nucleotides. In embodiments, the complement of the first independent single-stranded polynucleotide includes about 30 to about 100 nucleotides. In embodiments, the complement of the first independent single-stranded polynucleotide includes about 40 to about 100 nucleotides.

In embodiments, the complement of the second independent single-stranded polynucleotide includes about 5 to about 50 nucleotides. In embodiments, the complement of the second independent single-stranded polynucleotide includes about 10 to about 50 nucleotides. In embodiments, the complement of the second independent single-stranded polynucleotide includes about 15 to about 50 nucleotides. In embodiments, the complement of the second independent single-stranded polynucleotide includes about 20 to about 50 nucleotides. In embodiments, the complement of the second independent single-stranded polynucleotide includes about 10 to about 100 nucleotides. In embodiments, the complement of the second independent single-stranded polynucleotide includes about 20 to about 100 nucleotides. In embodiments, the complement of the second independent single-stranded polynucleotide includes about 30 to about 100 nucleotides. In embodiments, the complement of the second independent single-stranded polynucleotide includes about 40 to about 100 nucleotides.

In embodiments, the bridged polynucleotide has an extendable 3′ end. In embodiments, the bridged polynucleotide is extended by one or more polymerases. In embodiments, the bridged polynucleotide is extended by a DNA polymerase, or mutant thereof. In embodiments, the bridged polynucleotide is extended by an RNA polymerase, or mutant thereof. In embodiments, the bridged polynucleotide is extended by a reverse transcriptase, or mutant thereof.

In embodiments, the bridge oligonucleotide has an extendable 3′ end. In embodiments, the bridge oligonucleotide is extended by one or more polymerases. In embodiments, the bridge oligonucleotide is extended by a DNA polymerase, or mutant thereof. In embodiments, the bridge oligonucleotide is extended by an RNA polymerase, or mutant thereof. In embodiments, the bridge oligonucleotide is extended by a reverse transcriptase, or mutant thereof.

In embodiments, the overlap oligonucleotide has an extendable 3′ end. In embodiments, the overlap oligonucleotide is extended by one or more polymerases. In embodiments, the overlap oligonucleotide is extended by a DNA polymerase, or mutant thereof. In embodiments, the overlap oligonucleotide is extended by an RNA polymerase, or mutant thereof. In embodiments, the overlap oligonucleotide is extended by a reverse transcriptase, or mutant thereof.

In embodiments, the interposing oligonucleotide barcodes (alternatively referred to herein as interposing barcodes (IBCs)) provided herein include a first and second hybridization sequence that are complementary to a first and second sequence of a sample polynucleotide, respectively. In embodiments, each hybridization sequence includes about 10 to about 25 nucleotides (e.g., random nucleotides). In embodiments, each hybridization sequence includes about 3 to about 5 nucleotides (e.g., random nucleotides). In embodiments, each hybridization sequence has 3 to 5 nucleotides (e.g., random nucleotides). In embodiments, the first hybridization sequence includes more nucleotides than the second hybridization sequence. See for example FIG. 4 illustrating an interposing oligonucleotide barcode with asymmetric hybridization sequences. In embodiments, the first hybridization sequence includes about 3 to about 5 nucleotides (e.g., random nucleotides) and the second hybridization sequence includes about 3 to 25 nucleotides (e.g., random nucleotides). In embodiments, the first hybridization sequence includes about 3 to about 25 nucleotides and the second hybridization sequence includes about 3 to 5 nucleotides. In embodiments, the first hybridization sequence includes about 3 to about 25 nucleotides and the second hybridization sequence includes about 3 to 25 nucleotides. In embodiments, the first hybridization sequence includes about 10 to about 25 nucleotides and the second hybridization sequence includes about 10 to 5 nucleotides. In embodiments, the first hybridization sequence includes about 10 to about 15 nucleotides and the second hybridization sequence includes about 10 to 15 nucleotides. In embodiments, the interposing oligonucleotide barcodes provided herein include a hybridization sequence that includes about 1 to about 20 nucleotides, about 5 to about 15 nucleotides, or about 8 to about 12 nucleotides. In embodiments, the interposing oligonucleotide barcodes include a hybridization sequence that includes about 9 to about 18 nucleotides. In embodiments, the interposing oligonucleotide barcodes include a hybridization sequence that includes a targeted primer sequence, i.e. a nucleic acid sequence that is complementary to a known nucleic acid region. For example, the targeted primer sequence may be complementary to a universally conserved region, or complementary sequences to target specific genes or mutations that have relevancy to a particular cancer phenotype. In embodiments, the total combined length of the first hybridization sequence and the second hybridization sequence includes about 18 to about 25 nucleotides.

In embodiments, the interposing oligonucleotide barcodes provided herein include a hybridization sequence that includes about 1 to about 10 nucleotides, about 2 to about 9 nucleotides, about 3 to about 8 nucleotides, about 4 to about 7 nucleotides, or about 5 to about 6 nucleotides. In embodiments, the interposing oligonucleotide barcodes provided herein include a hybridization sequence that includes 1, 2, 3, 4, 5, 6, 7, 8, 9, or 10 nucleotides. In embodiments, the interposing oligonucleotide barcodes provided herein include a hybridization sequence that includes 3 nucleotides. In embodiments, the interposing oligonucleotide barcodes provided herein include a hybridization sequence that includes 4 nucleotides. In embodiments, the interposing oligonucleotide barcodes provided herein include a hybridization sequence that includes 5 nucleotides. In embodiments, the interposing oligonucleotide barcodes provided herein include a hybridization sequence that includes 6 nucleotides. In embodiments, the interposing oligonucleotide barcodes provided herein include a hybridization sequence that includes 7 nucleotides. In embodiments, the interposing oligonucleotide barcodes provided herein include a hybridization sequence that includes 8 nucleotides. In embodiments, the interposing oligonucleotide barcodes provided herein include two hybridization sequences, and each hybridization sequence consists of 4 nucleotides. In embodiments, the interposing oligonucleotide barcodes provided herein include two hybridization sequences, and each hybridization sequence consists of 5 nucleotides. In embodiments, the interposing oligonucleotide barcodes provided herein include two hybridization sequences, and each hybridization sequence consists of 6 nucleotides. In embodiments, the interposing oligonucleotide barcodes provided herein include two hybridization sequences, and each hybridization sequence consists of 7 nucleotides. In embodiments, the interposing oligonucleotide barcodes provided herein include two hybridization sequences, and each hybridization sequence consists of 8 nucleotides. In embodiments, the interposing oligonucleotide barcodes provided herein include two hybridization sequences, and each hybridization sequence consists of 9 nucleotides. In embodiments, the interposing oligonucleotide barcodes provided herein include two hybridization sequences, and each hybridization sequence consists of 10 nucleotides. In embodiments, the interposing oligonucleotide barcodes provided herein include two hybridization sequences, and each hybridization sequence consists of 11 nucleotides. In embodiments, the interposing oligonucleotide barcodes provided herein include two hybridization sequences, and each hybridization sequence consists of 12 nucleotides. In embodiments, the interposing oligonucleotide barcodes include a hybridization sequence having a first sequence (e.g., ATTG) and a second sequence (e.g., CCTA) that are independently different from each other. In embodiments, the interposing oligonucleotide barcodes include a hybridization sequence having a first sequence (e.g., TACG) and a second sequence (e.g., TACG) that are identical. In embodiments, the interposing oligonucleotide barcodes include a hybridization sequence having a first sequence (e.g., ATTG) and a second sequence (e.g., CCTATTACGATAACA (SEQ ID NO:2)) that are independently different from each other. In embodiments, the first hybridization sequence includes a targeted primer sequence, or a portion thereof. In embodiments, the second hybridization sequence includes a targeted priming sequence, or a portion thereof.

In embodiments, the hybridization sequence includes at least one target-specific region (also referred to herein as a target priming sequence). A target-specific region is a single stranded polynucleotide that is at least 50% complementary, at least 75% complementary, at least 85% complementary, at least 90% complementary, at least 95% complementary, at least 98%, at least 99% complementary, or 100% complementary to a portion of a nucleic acid molecule that includes a known target sequence (e.g., a gene or gene fragment of interest). In embodiments, the target-specific region is capable of hybridizing to at least a portion of the target sequence. In embodiments, the target-specific region is substantially non-complementary to other target sequences present in the sample.

The melting temperature (Tm) of an interposing barcode can be changed (e.g., increased) to a desired Tm using a suitable method, for example by changing (e.g., increasing) GC content, changing (e.g., increasing) length and/or by the inclusion of modified nucleotides, nucleotide analogues and/or modified nucleotides bonds, non-limiting examples of which include locked nucleic acids (LNAs, e.g., bicyclic nucleic acids), bridged nucleic acids (BNAs, e.g., constrained nucleic acids), C5-modified pyrimidine bases (for example, 5-methyl-dC, propynyl pyrimidines, among others) and alternate backbone chemistries, for example peptide nucleic acids (PNAs), morpholinos, the like or combinations thereof. In embodiments, the interposing barcodes include nucleotide analogues to increase binding stability (e.g., Locked Nucleic Acid bases (LNAs)). For example, an interposing barcode that includes synthetic analogue bases such as LNAs (e.g., LNAs as described in US 2003/0092905; U.S. Pat. No. 7,084,125, which are incorporated herein by reference for all purposes) may increase the Tm. In embodiments, the interposing barcode includes a plurality of LNAs (e.g., 2, 3, 4, 5, 6, 7, 8, 9, or 10 LNAs). In embodiments, the interposing barcode includes 2-6 LNAs. In embodiments, the hybridization sequence includes one or more modified nucleotides, such as LNAs. In embodiments, each hybridization sequence includes one or more LNAs. In embodiments, the interposing barcode has the general formula 5′-[hybridization sequence 1 domain]-[stem 1 domain]-[loop domain]-[stem 2 domain]-[hybridization sequence 2 domain]-3′. In embodiments, the interposing barcode has the formula: 5′Phos-[hybridization sequence 1 domain]-[stem 1 domain]-[loop domain]-[stem 2 domain]-[hybridization sequence 2 domain]-3′, wherein the hybridization sequence 1 domain has the sequence: ACCACG+GTCAC (SEQ ID NO:3); stem 1 domain has the sequence: CTCCAC (SEQ ID NO:4); loop domain has the sequence TTNNNNNNNNNNNNTT (SEQ ID NO: 5), wherein ‘N’ is a random nucleotide; stem 2 domain has the sequence: GTGGAG (SEQ ID NO: 6); and the hybridization sequence 2 domain has the sequence CGT+CTCCTCAG (SEQ ID NO:7), wherein +G and +C represent the LNA bases. In embodiments, the Tm of hybridization sequence is greater than 40° C. In embodiments, the Tm of hybridization sequence is greater than 45° C.

In embodiments, the interposing oligonucleotide barcodes provided herein include a first and second hybridization sequence that include randomly generated sequences. In embodiments, the interposing oligonucleotide barcodes provided herein include a first and second hybridization sequence that include targeting priming sequences, or a portion thereof. In embodiments, the interposing oligonucleotide barcodes provided herein do not include a first and second hybridization sequence that include randomly generated sequences.

In embodiments, the interposing oligonucleotide barcodes provided herein include a first and second stem region. The first and second stem regions are composed of complementary nucleotide sequences. In embodiments, the first stem region includes a sequence common to a plurality of the interposing oligonucleotide barcodes. In embodiments, the second stem region includes a sequence complementary to the first stem region, where the second stem region is capable of hybridizing to the first stem region under hybridization conditions.

In embodiments, the interposing oligonucleotide barcodes include a loop region that is comprised of random nucleotides, which may function as a molecular identifier. In embodiments, the loop region alone (e.g., Type 1 as observed in FIG. 1A) may be considered a molecular identifier. In embodiments, the loop region further includes a sample index sequence (e.g., Type 2 as observed in FIG. 1A).

In embodiments, the first and second stem regions of the interposing oligonucleotide barcodes provided herein include a known sequence of about 5 to about 10 nucleotides. In embodiments, the first and second stem regions of the interposing oligonucleotide barcodes provided herein include a known sequence of about 1 to about 20 nucleotides, about 2 to about 19, about 3 to about 18 nucleotides, about 4 to about 17 nucleotides, about 5 to about 16 nucleotides, about 6 to about 15 nucleotides, about 7 to about 14 nucleotides, about 8 to about 13 nucleotides, about 9 to about 12 nucleotides, or about 10 to about 11 nucleotides. In embodiments, the first and second stem regions of the interposing oligonucleotide barcodes provided herein include a known sequence of about 1, 2, 3, 4, 5, 6, 7, 8, 9, 10, 11, 12, 13, 14, 15, 16, 17, 18, 19, or about 20 nucleotides. In embodiments of the interposing oligonucleotide barcodes provided herein, the first stem region includes about 5 nucleotides. In embodiments of the interposing oligonucleotide barcodes provided herein, the first stem region includes about 6 nucleotides. In embodiments of the interposing oligonucleotide barcodes provided herein, the first stem region includes about 7 nucleotides. In embodiments of the interposing oligonucleotide barcodes provided herein, the first stem region includes about 8 nucleotides. In embodiments of the interposing oligonucleotide barcodes provided herein, the first stem region includes about 9 nucleotides. In embodiments of the interposing oligonucleotide barcodes provided herein, the first stem region includes about 10 nucleotides. In embodiments of the interposing oligonucleotide barcodes provided herein, the second stem region includes about 5 nucleotides. In embodiments of the interposing oligonucleotide barcodes provided herein, the second stem region includes about 6 nucleotides. In embodiments of the interposing oligonucleotide barcodes provided herein, the second stem region includes about 7 nucleotides. In embodiments of the interposing oligonucleotide barcodes provided herein, the second stem region includes about 8 nucleotides. In embodiments of the interposing oligonucleotide barcodes provided herein, the second stem region includes about 9 nucleotides. In embodiments of the interposing oligonucleotide barcodes provided herein, the second stem region includes about 10 nucleotides. In embodiments, the first and second stem regions are substantially complementary to each other.

In embodiments, the interposing oligonucleotide barcodes provided herein include a loop region that further includes a sample index sequence. In general, a sample index sequence is the same for all polynucleotides from the same sample source (e.g., the same subject, the same aliquot, or the same container), and differs from the sample index sequence of polynucleotides from a different sample source. Polynucleotides from different samples can therefore be mixed, and the sequences subsequently grouped by sample source by virtue of the sample index sequence. In embodiments, the sample index sequence is a randomly generated sequence that is sufficiently different from other sample index sequences to allow the identification of the sample source based on index sequence(s) with which they are associated. In embodiments, each sample index sequence in a plurality of index sequences differs from every other index sequence in the plurality by at least three nucleotide positions, such as at least 3, 4, 5, 6, 7, 8, 9, 10, or more nucleotide positions. In some embodiments, substantially degenerate index sequences may be known as random. In some embodiments a sample index sequence may include a nucleic acid sequence from within a pool of known sequences. In some embodiments, the sample index sequences may be pre-defined. In embodiments, the sample index sequence includes about 1 to about 10 nucleotides. In embodiments, the sample index sequence includes about 3, 4, 5, 6, 7, 8, 9, or about 10 nucleotides. In embodiments, the sample index sequence includes about 3 nucleotides. In embodiments, the sample index sequence includes about 5 nucleotides. In embodiments, the sample index sequence includes about 7 nucleotides. In embodiments, the sample index sequence includes about 10 nucleotides. In embodiments, the sample index sequence includes about 11 nucleotides. In embodiments, the sample index sequence includes about 12 nucleotides. In embodiments, the sample index sequence includes about 8 to 15 nucleotides. In embodiments, the sample index sequence includes 12 nucleotides.

In embodiments, the interposing oligonucleotide barcodes provided herein include a loop region. In embodiments, the loop region, alone or in combination with a sequence of one or both of (a) the sample polynucleotide, or (b) one or more barcode sequences, uniquely distinguishes the sample polynucleotide from other sample polynucleotides in a plurality of sample polynucleotides. In embodiments of the interposing oligonucleotide barcodes provided herein, the loop region includes about 5 to about 20 nucleotides or about 10 to about 20 nucleotides. In embodiments of the interposing oligonucleotide barcodes provided herein, the loop region includes about 1 to about 25, about 2 to about 24, about 3 to about 23, about 4 to about 22, about 5 to about 21, about 6 to about 20, about 7 to about 19, about 8 to about 18, about 9 to about 17, about 10 to about 16, about 11 to about 15, or about 12 to about 14 nucleotides. In embodiments of the interposing oligonucleotide barcodes provided herein, the loop region includes about 1, 2, 3, 4, 5, 6, 7, 8, 9, 10, 11, 12, 13, 14, 15, 16, 17, 18, 19, 20, 21, 22, 23, 24, or about 25 nucleotides. In embodiments of the interposing oligonucleotide barcodes provided herein, the loop region includes about 5 nucleotides. In embodiments of the interposing oligonucleotide barcodes provided herein, the loop region includes about 10 nucleotides. In embodiments of the interposing oligonucleotide barcodes provided herein, the loop region includes about 15 nucleotides. In embodiments of the interposing oligonucleotide barcodes provided herein, the loop region includes about 20 nucleotides. In embodiments, the loop region does not include a sample index sequence. In embodiments, the loop includes a TT-[UMI sequence]-TT sequence, such as TT-[NNNNNNNNNNNN]-TT (SEQ ID NO:1) sequence, wherein N represents random nucleotides and A, T, C, G represent fixed nucleotides).

In embodiments, the interposing oligonucleotide barcodes provided herein include a loop region that includes a barcode sequence. In embodiments, the loop includes only one barcode (e.g., one UMI sequence). In embodiments, the barcode sequence, alone or in combination with a sequence of one or both of (a) the sample polynucleotide, or (b) one or more additional barcode sequences, uniquely distinguishes the sample polynucleotide from other sample polynucleotides in a plurality of sample polynucleotides. In embodiments of the interposing oligonucleotide barcodes provided herein, the barcode sequence includes about 5 to about 20 nucleotides or about 10 to about 20 nucleotides. In embodiments of the interposing oligonucleotide barcodes provided herein, the barcode sequence includes about 1 to about 25, about 2 to about 24, about 3 to about 23, about 4 to about 22, about 5 to about 21, about 6 to about 20, about 7 to about 19, about 8 to about 18, about 9 to about 17, about 10 to about 16, about 11 to about 15, or about 12 to about 14 nucleotides. In embodiments of the interposing oligonucleotide barcodes provided herein, the barcode sequence includes about 1, 2, 3, 4, 5, 6, 7, 8, 9, 10, 11, 12, 13, 14, 15, 16, 17, 18, 19, 20, 21, 22, 23, 24, or about 25 nucleotides. In embodiments of the interposing oligonucleotide barcodes provided herein, the barcode sequence includes about 5 nucleotides. In embodiments of the interposing oligonucleotide barcodes provided herein, the barcode sequence includes about 10 nucleotides. In embodiments of the interposing oligonucleotide barcodes provided herein, the barcode sequence includes about 15 nucleotides. In embodiments of the interposing oligonucleotide barcodes provided herein, the barcode sequence includes about 20 nucleotides. In embodiments, the loop region does not include a barcode sequence.

In embodiments, the interposing oligonucleotide barcodes provided herein include a loop region that includes a barcode sequence, wherein the barcode sequence is selected from a set of barcode sequences represented by a random or partially random sequence. In embodiments, the interposing oligonucleotide barcodes provided herein include a loop region that includes a barcode sequence, where the barcode sequence is selected from a set of barcode sequences represented by a random sequence. In embodiments, the interposing oligonucleotide barcodes provided herein include a loop region that includes a barcode sequence, where each barcode sequence is selected from a set of barcode sequences represented by a partially random sequence.

In embodiments, the interposing oligonucleotide barcodes provided herein includes a random sequence. In embodiments, the interposing oligonucleotide barcodes provided herein include a barcode sequence that includes a random sequence. In embodiments, the random sequence excludes a subset of sequences, where the excluded subset includes sequences with three or more identical consecutive nucleotides. In embodiments, the excluded subset includes sequences with three identical consecutive nucleotides. In embodiments, the excluded subset includes sequences with four identical consecutive nucleotides. In embodiments, the excluded subset includes sequences with five identical consecutive nucleotides.

In embodiments, the interposing oligonucleotide barcodes provided herein include a barcode sequence, where each barcode sequence differs from every other barcode sequence by at least two nucleotide positions. In embodiments, the interposing oligonucleotide barcodes provided herein include barcode sequences, where each barcode sequence differs from every other barcode sequence by at least three nucleotide positions. In embodiments, the interposing oligonucleotide barcodes provided herein include barcode sequences, where each barcode sequence differs from every other barcode sequence by at least four nucleotide positions. In embodiments, the interposing oligonucleotide barcodes provided herein include barcode sequences, where each barcode sequence differs from every other barcode sequence by at least five nucleotide positions.

In embodiments, the interposing oligonucleotide barcodes provided herein include a loop region that includes a barcode sequence that alone or in combination with a sequence of one or both of (a) the sample polynucleotide, or (b) one or more additional barcode sequences, uniquely distinguishes the sample polynucleotide from other sample polynucleotides in a plurality of sample polynucleotides. In embodiments, the interposing oligonucleotide barcodes provided herein include a loop region that includes a barcode sequence that alone uniquely distinguishes the sample polynucleotide from other sample polynucleotides in a plurality of sample polynucleotides. In embodiments, the interposing oligonucleotide barcodes provided herein include a loop region that includes a barcode sequence that in combination with a sequence of the sample polynucleotide uniquely distinguishes the sample polynucleotide from other sample polynucleotides in a plurality of sample polynucleotides. In embodiments, the interposing oligonucleotide barcodes provided herein include a loop region that includes a barcode sequence that in combination with a sequence of one or more additional barcode sequences, uniquely distinguishes the sample polynucleotide from other sample polynucleotides in a plurality of sample polynucleotides. In embodiments, the interposing oligonucleotide barcodes provided herein include a loop region that includes a barcode sequence that in combination with a sequence of the sample polynucleotide, and one or more additional barcode sequences, uniquely distinguishes the sample polynucleotide from other sample polynucleotides in a plurality of sample polynucleotides.

In embodiments, the interposing oligonucleotide barcodes provided herein include a 5′ phosphate moiety. A phosphate moiety attached to the 5′-end permits ligation of two nucleotides, i.e., the covalent binding of a 5′-phosphate to the 3′-hydroxyl group of another nucleotide, to form a phosphodiester bond. Removal of the 5′-phosphate prevents ligation.

In embodiments, provided herein is a composition including a sample polynucleotide hybridized to a plurality of oligonucleotides barcodes (e.g., interposing barcodes) according to any of the aspects of interposing barcodes described herein. In embodiments the sample polynucleotide is an RNA transcript. In embodiments, the polynucleotide is mRNA.

In embodiments, provided herein is a composition including a sample polynucleotide hybridized to a plurality of oligonucleotides barcodes (e.g., interposing barcodes) according to any of the aspects of interposing barcodes described herein, where the second hybridization sequence is at least twice as long as the first hybridization sequence (e.g., the first hybridization sequence is 5 nucleotides in length and the second is at least 10 nucleotides in length). In embodiments, the second hybridization sequence is at least three times as long as the first hybridization sequence. In embodiments, the second hybridization sequence is at least four times as long as the first hybridization sequence. In embodiments, the second hybridization sequence is more than four times as long as the first hybridization sequence. In embodiments, the second hybridization sequence is the same length as the first hybridization sequence. In embodiments, the sample polynucleotide can include any nucleic acid of interest. The nucleic acid can include DNA, RNA, peptide nucleic acid (PNA), morpholino nucleic acid, locked nucleic acid (LNA), glycol nucleic acid, threose nucleic acid, mixtures thereof, and hybrids thereof. In embodiments, the nucleic acid is obtained from one or more source organisms. In some embodiments, the nucleic acid can include a selected sequence or a portion of a larger sequence. In embodiments, sequencing a portion of a nucleic acid or a fragment thereof can be used to identify the source of the nucleic acid. With reference to nucleic acids, polynucleotides and/or nucleotide sequences a “portion,” “fragment” or “region” can be at least 5 consecutive nucleotides, at least 10 consecutive nucleotides, at least 15 consecutive nucleotides, at least 20 consecutive nucleotides, at least 25 consecutive nucleotides, at least 50 consecutive nucleotides, at least 100 consecutive nucleotides, or at least 150 consecutive nucleotides.

In embodiments, the sample polynucleotide is at least 1000 bases (1 kb), at least 2 kb, at least 4 kb, at least 6 kb, at least 10 kb, at least 20 kb, at least 30 kb, at least 40 kb, or at least 50 kb in length. In embodiments, the entire sequence of the sample polynucleotide is about 1 to 3 kb, and only a portion of that the sample polynucleotide (e.g., 50 to 100 nucleotides) is sequenced at a time. In embodiments, the sample polynucleotide is about 2 to 3 kb. In embodiments, the sample polynucleotide is about 1 to 10 kb. In embodiments, the sample polynucleotide is about 3 to 10 kb. In embodiments, the sample polynucleotide is about 5 to 10 kb. In embodiments, the sample polynucleotide is about 1 to 3 kb. In embodiments, the sample polynucleotide is about 1 to 2 kb. In embodiments, the sample polynucleotide is greater than 1 kb. In embodiments, the sample polynucleotide is greater than 500 bases. In embodiments, the sample polynucleotide is about 1 kb. In embodiments, the sample polynucleotide is about 2 kb. In embodiments, the sample polynucleotide is less than 1 kb. In embodiments, the sample polynucleotide is about 500 nucleotides. In embodiments, the sample polynucleotide is about 510 nucleotides. In embodiments, the sample polynucleotide is about 520 nucleotides. In embodiments, the sample polynucleotide is about 530 nucleotides. In embodiments, the sample polynucleotide is about 540 nucleotides. In embodiments, the sample polynucleotide is about 550 nucleotides. In embodiments, the sample polynucleotide is about 560 nucleotides. In embodiments, the sample polynucleotide is about 570 nucleotides. In embodiments, the sample polynucleotide is about 580 nucleotides. In embodiments, the sample polynucleotide is about 590 nucleotides. In embodiments, the sample polynucleotide is about 600 nucleotides. In embodiments, the sample polynucleotide is about 610 nucleotides. In embodiments, the sample polynucleotide is about 620 nucleotides. In embodiments, the sample polynucleotide is about 630 nucleotides. In embodiments, the sample polynucleotide is about 640 nucleotides. In embodiments, the sample polynucleotide is about 650 nucleotides. In embodiments, the sample polynucleotide is about 660 nucleotides. In embodiments, the sample polynucleotide is about 670 nucleotides. In embodiments, the sample polynucleotide is about 680 nucleotides. In embodiments, the sample polynucleotide is about 690 nucleotides. In embodiments, the sample polynucleotide is about 700 nucleotides. In embodiments, the sample polynucleotide is about 1,600 nucleotides. In embodiments, the sample polynucleotide is about 1,610 nucleotides. In embodiments, the sample polynucleotide is about 1,620 nucleotides. In embodiments, the sample polynucleotide is about 1,630 nucleotides. In embodiments, the sample polynucleotide is about 1,640 nucleotides. In embodiments, the sample polynucleotide is about 1,650 nucleotides. In embodiments, the sample polynucleotide is about 1,660 nucleotides. In embodiments, the sample polynucleotide is about 1,670 nucleotides. In embodiments, the sample polynucleotide is about 1,680 nucleotides. In embodiments, the sample polynucleotide is about 1,690 nucleotides. In embodiments, the sample polynucleotide is about 1,700 nucleotides. In embodiments, the sample polynucleotide is about 1,710 nucleotides. In embodiments, the sample polynucleotide is about 1,720 nucleotides. In embodiments, the sample polynucleotide is about 1,730 nucleotides. In embodiments, the sample polynucleotide is about 1,740 nucleotides. In embodiments, the sample polynucleotide is about 1,750 nucleotides. In embodiments, the sample polynucleotide is about 1,760 nucleotides. In embodiments, the sample polynucleotide is about 1,770 nucleotides. In embodiments, the sample polynucleotide is about 1,780 nucleotides. In embodiments, the sample polynucleotide is about 1,790 nucleotides. In embodiments, the sample polynucleotide is about 1,800 nucleotides.

In embodiments, the sample polynucleotide is a nucleic acid sequence. In embodiments the sample polynucleotide is an RNA transcript. RNA transcripts are responsible for the process of converting DNA into an organism's phenotype, thus by determining the types and quantity of RNA present in a sample (e.g., a cell), it is possible to assign a phenotype to the cell. RNA transcripts include coding RNA and non-coding RNA molecules, such as messenger RNA (mRNA), transfer RNA (tRNA), micro RNA (miRNA), small interfering RNA (siRNA), small nucleolar RNA (snoRNA), small nuclear RNA (snRNA), Piwi-interacting RNA (piRNA), enhancer RNA (eRNA), or ribosomal RNA (rRNA). In embodiments, the target is pre-mRNA. In embodiments, the target is heterogeneous nuclear RNA (hnRNA). In embodiments the sample polynucleotide is a single stranded RNA nucleic acid sequence. In embodiments, the sample polynucleotide is an RNA nucleic acid sequence or a DNA nucleic acid sequence (e.g., cDNA). In embodiments, the sample polynucleotide is a cDNA target nucleic acid sequence.

In embodiments, the sample polynucleotides are RNA nucleic acid sequences or DNA nucleic acid sequences. In embodiments, the sample polynucleotides are RNA nucleic acid sequences or DNA nucleic acid sequences from the same cell. In embodiments, the sample polynucleotides are RNA nucleic acid sequences. In embodiments, the RNA nucleic acid sequence is stabilized using known techniques in the art. For example, RNA degradation by RNase should be minimized using commercially available solutions (e.g., RNA Later®, RNA Protect®, or DNA/RNA Shield®). In embodiments, the sample polynucleotides are messenger RNA (mRNA), transfer RNA (tRNA), micro RNA (miRNA), small interfering RNA (siRNA), small nucleolar RNA (snoRNA), small nuclear RNA (snRNA), Piwi-interacting RNA (piRNA), enhancer RNA (eRNA), or ribosomal RNA (rRNA). In embodiments, the sample polynucleotide is pre-mRNA. In embodiments, the sample polynucleotide is heterogeneous nuclear RNA (hnRNA). In embodiments, the sample polynucleotide is mRNA, tRNA (transfer RNA), rRNA (ribosomal RNA), or noncoding RNA (such as lncRNA (long noncoding RNA)). In embodiments, the sample polynucleotides are on different regions of the same RNA nucleic acid sequence. In embodiments, the sample polynucleotides are cDNA target nucleic acid sequences and before step i), the RNA nucleic acid sequences are reverse transcribed to generate the cDNA target nucleic acid sequences. In embodiments, the sample polynucleotides are not reverse transcribed to cDNA. When mRNA is reverse transcribed an oligo(dT) primer can be added to better hybridize to the poly A tail of the mRNA. The oligo(dT) primer may include between about 12 and about 25 dT residues. The oligo(dT) primer may be an oligo(dT) primer of between about 18 to about 25 nt in length.

In embodiments, the polynucleotide includes a gene or a gene fragment. In embodiments, the gene or gene fragment is a cancer-associated gene or fragment thereof, T cell receptor (TCRs) gene or fragment thereof, or a B cell receptor (BCRs) gene, or fragment thereof. In embodiments, the gene or gene fragment is a CDR3 gene or fragment thereof. In embodiments, the gene or gene fragment is a T cell receptor alpha variable (TRAV) gene or fragment thereof, T cell receptor alpha joining (TRAJ) gene or fragment thereof, T cell receptor alpha constant (TRAC) gene or fragment thereof, T cell receptor beta variable (TRBV) gene or fragment thereof, T cell receptor beta diversity (TRBD) gene or fragment thereof, T cell receptor beta joining (TRBJ) gene or fragment thereof, T cell receptor beta constant (TRBC) gene or fragment thereof, T cell receptor gamma variable (TRGV) gene or fragment thereof, T cell receptor gamma joining (TRGJ) gene or fragment thereof, T cell receptor gamma constant (TRGC) gene or fragment thereof, T cell receptor delta variable (TRDV) gene or fragment thereof, T cell receptor delta diversity (TRDD) gene or fragment thereof, T cell receptor delta joining (TRDJ) gene or fragment thereof, or T cell receptor delta constant (TRDC) gene or fragment thereof.

In embodiments, the methods and compositions described herein are utilized to analyze the various sequences of T cell receptors (TCRs) and B cell receptors (BCRs) from immune cells, for example various clonotypes. In embodiments, the target nucleic acid includes a nucleic acid sequence encoding a TCR alpha (TCRA) chain, a TCR beta (TCRB) chain, a TCR delta (TCRD) chain, a TCR gamma (TCRG) chain, or any fragment thereof (e.g., variable regions including VDJ or VJ regions, constant regions, transmembrane regions, fragments thereof, combinations thereof, and combinations of fragments thereof). In embodiments, the target nucleic acid includes a nucleic acid sequence encoding a B cell receptor heavy chain, B cell receptor light chain, or any fragment thereof (e.g., variable regions including VDJ or VJ regions, constant regions, transmembrane regions, fragments thereof, combinations thereof, and combinations of fragments thereof). In embodiments, the target nucleic acid includes a CDR3 nucleic acid sequence. In embodiments, the target nucleic acid includes a TCRA gene sequence or a TCRB gene sequence. In embodiments, the target nucleic acid includes a TCRA gene sequence and a TCRB gene sequence. In embodiments, the target nucleic acid includes sequences of various T cell receptor alpha variable genes (TRAV genes), T cell receptor alpha joining genes (TRAJ genes), T cell receptor alpha constant genes (TRAC genes), T cell receptor beta variable genes (TRBV genes), T cell receptor beta diversity genes (TRBD genes), T cell receptor beta joining genes (TRBJ genes), T cell receptor beta constant genes (TRBC genes), T cell receptor gamma variable genes (TRGV genes), T cell receptor gamma joining genes (TRGJ genes), T cell receptor gamma constant genes (TRGC genes), T cell receptor delta variable genes (TRDV genes), T cell receptor delta diversity genes (TRDD genes), T cell receptor delta joining genes (TRDJ genes), or T cell receptor delta constant genes (TRDC genes).

In an aspect is provided a kit including the bridged polynucleotide and the plurality of interposing oligonucleotide adapters as described herein. In an aspect is provided a kit including the bridging polynucleotide and the plurality of interposing oligonucleotide probes as described herein.

In an aspect is provided a kit including: i. a plurality of interposing oligonucleotide probes capable of hybridizing to a bridged polynucleotide, the interposing oligonucleotide probes including from 5′ to 3′: i. a first hybridization sequence complementary to a first sequence of the bridged polynucleotide; ii. a loop region including a primer binding sequence; and iii. a second hybridization sequence complementary to a second sequence of the bridged polynucleotide; ii. a plurality of 5′ terminal oligonucleotide probes capable of hybridizing to a bridged polynucleotide, the 5′ terminal oligonucleotide probes including from 5′ to 3′: i. a hybridization sequence complementary to a 5′ terminal sequence of the bridged polynucleotide, wherein the 5′ terminal sequence is upstream of the bridged polynucleotide sequence complementary to the interposing oligonucleotide probes; and ii. a primer binding sequence; iii. a plurality of 3′ terminal oligonucleotide probes capable of hybridizing to a bridged polynucleotide, the 3′ terminal oligonucleotide probes including from 3′ to 5′: i. a hybridization sequence complementary to a 3′ terminal sequence of the bridged polynucleotide, wherein the 3′ terminal sequence is downstream of the bridged polynucleotide sequence complementary to the interposing oligonucleotide probes; and ii. a primer binding sequence; and iv. a bridging oligonucleotide including from 5′ to 3′: i. a hybridization sequence complementary to a 3′ terminal sequence of a first independent polynucleotide; ii. a linker sequence; and iii. a hybridization sequence complementary to a 5′ terminal sequence of a second independent polynucleotide.

In an aspect is provided a kit including: i. a plurality of interposing oligonucleotide probes capable of hybridizing to a bridged polynucleotide, the interposing oligonucleotide probes including from 5′ to 3′: i. a first hybridization sequence complementary to a first sequence of the bridged polynucleotide; ii. a loop region including a primer binding sequence; and iii. a second hybridization sequence complementary to a second sequence of the bridged polynucleotide; ii. a plurality of 5′ terminal oligonucleotide probes capable of hybridizing to a bridged polynucleotide, the 5′ terminal oligonucleotide probes including from 5′ to 3′: i. a hybridization sequence complementary to a 5′ terminal sequence of the bridged polynucleotide, wherein the 5′ terminal sequence is upstream of the bridged polynucleotide sequence complementary to the interposing oligonucleotide probes; and ii. a primer binding sequence; iii. a plurality of 3′ terminal oligonucleotide probes capable of hybridizing to a bridged polynucleotide, the 3′ terminal oligonucleotide probes including from 3′ to 5′: i. a hybridization sequence complementary to a 3′ terminal sequence of the bridged polynucleotide, wherein the 3′ terminal sequence is downstream of the bridged polynucleotide sequence complementary to the interposing oligonucleotide probes; and ii. a primer binding sequence; iv. a first overlap oligonucleotide including from 3′ to 5′: i. a hybridization sequence complementary to a 3′ sequence of a first independent polynucleotide; and ii. a hybridization sequence complementary to a 5′ sequence of a second overlap oligonucleotide; and v. the second overlap oligonucleotide including from 3′ to 5′: i. a hybridization sequence complementary to a 3′ sequence of a second independent polynucleotide; and ii. a hybridization sequence complementary to a 5′ sequence of the first overlap oligonucleotide.

In embodiments, the kit includes one or more first overlap oligonucleotides. In embodiments, the kit includes one or more second overlap oligonucleotides. In embodiments, the kit includes one or more first overlap oligonucleotides hybridized to one or more second overlap oligonucleotides. In embodiments, the kit includes one or more first overlap oligonucleotides, one or more second overlap oligonucleotides, and one or more bridge oligonucleotides.

Generally, the kit includes one or more containers providing a composition and one or more additional reagents (e.g., a buffer suitable for polynucleotide extension). The kit may also include a template nucleic acid (DNA and/or RNA), one or more primer polynucleotides, nucleoside triphosphates (including, e.g., deoxyribonucleotides, ribonucleotides, labeled nucleotides, and/or modified nucleotides), buffers, salts, and/or labels (e.g., fluorophores). In embodiments, the kit includes components useful for ligating polynucleotides using a ligation enzyme (e.g., CircLigase™ enzyme, Taq DNA Ligase, HiFi Taq DNA Ligase, T4 DNA ligase, T4 RNA ligase, T4 RNA ligase 2, or Ampligase® DNA Ligase). For example, such a kit further includes the following components: (a) reaction buffer for controlling pH and providing an optimized salt composition for a ligation enzyme (e.g., CircLigase™ enzyme, Taq DNA Ligase, HiFi Taq DNA Ligase, T4 DNA ligase, T4 RNA ligase 2, or Ampligase® DNA Ligase), and (b) ligation enzyme cofactors, such as ATP and a divalent ion (e.g., Mn²⁺ or Mg²⁺) In embodiments, the kit includes components useful for performing second-strand cDNA synthesis (e.g., a RNAse H enzyme, a DNA Polymerase I enzyme, and a ligation enzyme such as T4 DNA ligase).

In embodiments, the polymerase in the kit is a bacterial DNA polymerase, eukaryotic DNA polymerase, archaeal DNA polymerase, viral DNA polymerase, or phage DNA polymerases. Bacterial DNA polymerases include E. coli DNA polymerases I, II and III, IV and V, the Klenow fragment of E. coli DNA polymerase, Clostridium stercorarium (Cst) DNA polymerase, Clostridium thermocellum (Cth) DNA polymerase and Sulfolobus solfataricus (Sso) DNA polymerase. Eukaryotic DNA polymerases include DNA polymerases α, β, γ, ε, €, η, ζ, λ, σ, μ, and k, as well as the Revl polymerase (terminal deoxycytidyl transferase) and terminal deoxynucleotidyl transferase (TdT). Viral DNA polymerases include T4 DNA polymerase, phi-29 DNA polymerase, GA-1, phi-29-like DNA polymerases, PZA DNA polymerase, phi-15 DNA polymerase, Cpl DNA polymerase, Cpl DNA polymerase, T7 DNA polymerase, and T4 polymerase. Other useful DNA polymerases include thermostable and/or thermophilic DNA polymerases such as Thermus aquaticus (Taq) DNA polymerase, Thermus filiformis (Tfi) DNA polymerase, Thermococcus zilligi (Tzi) DNA polymerase, Thermus thermophilus (Tth) DNA polymerase, Thermus flavusu (Tfl) DNA polymerase, Pyrococcus woesei (Pwo) DNA polymerase, Pyrococcus furiosus (Pfu) DNA polymerase and Turbo Pfu DNA polymerase, Thermococcus litoralis (Tli) DNA polymerase, Pyrococcus sp. GB-D polymerase, Thermotoga maritima (Tma) DNA polymerase, Bacillus stearothermophilus (Bst) DNA polymerase, Pyrococcus Kodakaraensis (KOD) DNA polymerase, Pfx DNA polymerase, Thermococcus sp. JDF-3 (JDF-3) DNA polymerase, Thermococcus gorgonarius (Tgo) DNA polymerase, Thermococcus acidophilium DNA polymerase; Sulfolobus acidocaldarius DNA polymerase; Thermococcus sp. go N-7 DNA polymerase; Pyrodictium occultum DNA polymerase; Methanococcus voltae DNA polymerase; Methanococcus thermoautotrophicum DNA polymerase; Methanococcus jannaschii DNA polymerase; Desulfurococcus strain TOK DNA polymerase (D. Tok Pol); Pyrococcus abyssi DNA polymerase; Pyrococcus horikoshii DNA polymerase; Pyrococcus islandicum DNA polymerase; Thermococcus fumicolans DNA polymerase; Aeropyrum pernix DNA polymerase; and the heterodimeric DNA polymerase DP1/DP2. In embodiments, the polymerase is 3PDX polymerase as disclosed in U.S. Pat. No. 8,703,461, the disclosure of which is incorporated herein by reference. In embodiments, the polymerase is a reverse transcriptase. Exemplary reverse transcriptases include, but are not limited to, HIV-1 reverse transcriptase from human immunodeficiency virus type 1 (PDB 1HMV), HIV-2 reverse transcriptase from human immunodeficiency virus type 2, M-MLV reverse transcriptase from the Moloney murine leukemia virus, AMV reverse transcriptase from the avian myeloblastosis virus, or Telomerase reverse transcriptase. In embodiments, the polymerase is a mutant P. abyssi polymerase (e.g., such as a mutant P. abyssi polymerase described in WO 2018/148723 or WO 2020/056044, each of which are incorporated herein by reference for all purposes). In embodiments, the kit includes a strand-displacing polymerase. In embodiments, the kit includes a strand-displacing polymerase, such as a phi29 polymerase, phi29 mutant polymerase or a thermostable phi29 mutant polymerase.

In embodiments, the kit includes a buffered solution. Typically, the buffered solutions contemplated herein are made from a weak acid and its conjugate base or a weak base and its conjugate acid. For example, sodium acetate and acetic acid are buffer agents that can be used to form an acetate buffer. Other examples of buffer agents that can be used to make buffered solutions include, but are not limited to, Tris, Bicine, Tricine, HEPES, TES, MOPS, MOPSO and PIPES. Additionally, other buffer agents that can be used in enzyme reactions, hybridization reactions, and detection reactions are known in the art. In embodiments, the buffered solution can include Tris. With respect to the embodiments described herein, the pH of the buffered solution can be modulated to permit any of the described reactions. In some embodiments, the buffered solution can have a pH greater than pH 7.0, greater than pH 7.5, greater than pH 8.0, greater than pH 8.5, greater than pH 9.0, greater than pH 9.5, greater than pH 10, greater than pH 10.5, greater than pH 11.0, or greater than pH 11.5. In other embodiments, the buffered solution can have a pH ranging, for example, from about pH 6 to about pH 9, from about pH 8 to about pH 10, or from about pH 7 to about pH 9. In embodiments, the buffered solution can include one or more divalent cations. Examples of divalent cations can include, but are not limited to, Mg2+, Mn2+, Zn2+, and Ca2+. In embodiments, the buffered solution can contain one or more divalent cations at a concentration sufficient to permit hybridization of a nucleic acid. In embodiments, the buffer includes PEG (polyethylene glycol), PVP (polyvinylpyrrolidone), trehalose, ficoll, or dextran. In embodiments, the buffer includes additives such as Tween-20 or NP-40.

In embodiments, the kit includes a sequencing reaction mixture. As used herein, the term “sequencing reaction mixture” is used in accordance with its plain and ordinary meaning and refers to an aqueous mixture that contains the reagents necessary to allow a nucleotide or nucleotide analogue to be added to a DNA strand by a DNA polymerase.

Adapters, interposing oligonucleotide barcodes, and/or primers may be supplied in the kits ready for use, or more preferably as concentrates-requiring dilution before use, or even in a lyophilized or dried form requiring reconstitution prior to use. If required, the kits may further include a supply of a suitable diluent for dilution or reconstitution of the primers. Optionally, the kits may further include supplies of reagents, buffers, enzymes, and dNTPs for use in carrying out nucleic acid amplification and/or sequencing. Further components which may optionally be supplied in the kit include sequencing primers suitable for sequencing templates prepared using the methods described herein.

### III. Methods

In an aspect is provided a method of amplifying a tagged complement of two independent single-stranded polynucleotides, the method including: a. hybridizing a bridge oligonucleotide to a first polynucleotide and a second polynucleotide, thereby forming a bridged polynucleotide complex; b. hybridizing one or more interposing oligonucleotide probes to the first polynucleotide and second polynucleotide, wherein each of the interposing oligonucleotide probes includes from 5′ to 3′: i. a first hybridization sequence complementary to a first sequence of the first polynucleotide and second polynucleotide; ii. a loop region including a primer binding sequence and optionally a barcode; and iii. a second hybridization sequence complementary to a second sequence of first polynucleotide and second polynucleotide; c. extending the 3′ end of each second hybridization sequence of the interposing oligonucleotide probes and the 3′ end of the hybridization sequence of the bridge oligonucleotide with one or more polymerases thereby forming an extension product of each of the oligonucleotide probes; d. ligating the 3′ end of each of the extension products to the 5′ end of the adjacent extension products, thereby making an integrated strand including a complement of the template nucleic acid including a plurality of the oligonucleotide probes; and e. amplifying the integrated strand by an amplification reaction to produce a tagged complement of two independent single-stranded polynucleotides.

In embodiments, the bridge oligonucleotide includes, from 5′ to 3′, a first hybridization sequence complementary to a 3′ sequence of the first independent polynucleotide, and a second hybridization sequence complementary to a 5′ sequence of the second independent polynucleotide.

In an aspect is provided a method of amplifying a tagged complement of two independent single-stranded polynucleotides, the method including: a. hybridizing a first overlap oligonucleotide to the first independent polynucleotide and a second overlap oligonucleotide to the second independent polynucleotide, and extending both the first and second overlap oligonucleotides with a polymerase, thereby forming an overlapped polynucleotide complex, wherein the overlapped polynucleotide complex includes a complement of the first independent polynucleotide, the first overlap oligonucleotide, the second overlap oligonucleotide, and a complement of the second independent polynucleotide, wherein a 5′ sequence of the first overlap oligonucleotide is hybridized to a 5′ sequence of the second overlap oligonucleotide; b. linking the overlapped polynucleotide complex, thereby generating a bridged polynucleotide including a sequence of the first independent polynucleotide linked to a sequence of the second independent polynucleotide or a complement thereof, c. hybridizing to the bridged polynucleotide one or more interposing oligonucleotide barcodes, wherein each of the interposing oligonucleotide barcodes includes from 5′ to 3′: i. a first hybridization sequence complementary to a first sequence of the bridged polynucleotide; ii. a loop region including a barcode; and iii. a second hybridization sequence complementary to a second sequence of the bridged polynucleotide; d. extending the 3′ end of each second hybridization sequence of the interposing oligonucleotide barcodes with one or more polymerases thereby forming an extension product of each of the interposing oligonucleotide barcodes; e. ligating the 3′ end of each of the extension products to the 5′ end of the adjacent extension products hybridized to the bridged polynucleotide thereby making an integrated strand tagged with a plurality of interposing oligonucleotide barcodes, wherein the integrated strand includes sequences of the first and second independent polynucleotides or complements thereof; and f. amplifying the integrated strand by an amplification reaction to produce a tagged complement of two independent single-stranded polynucleotides.

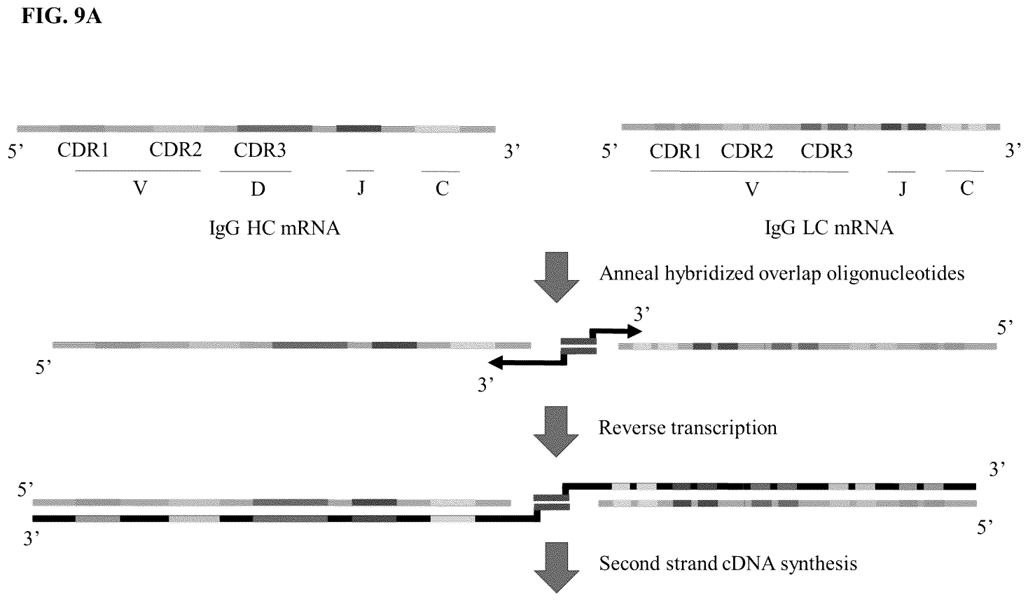

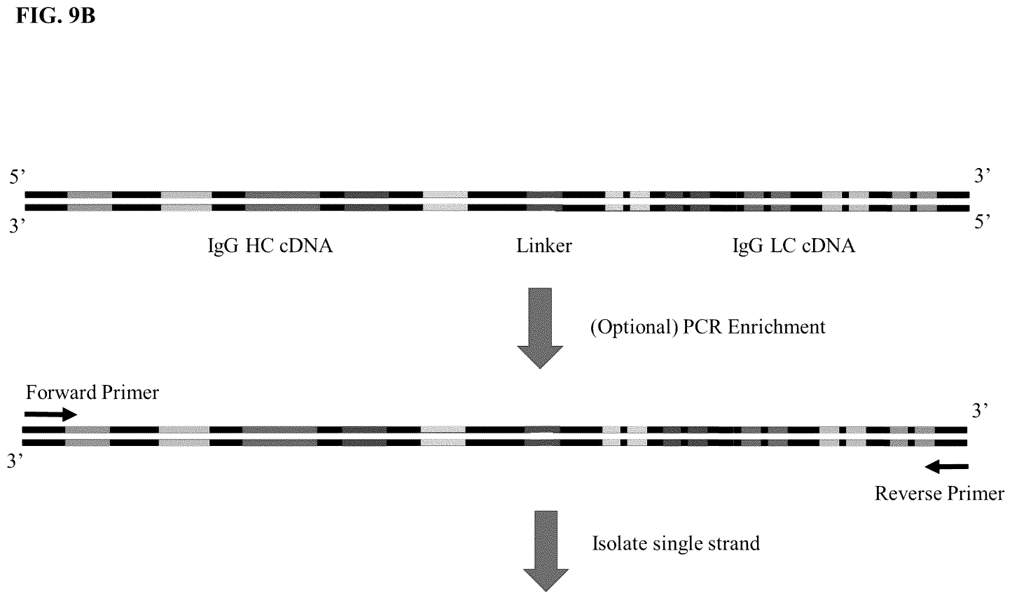

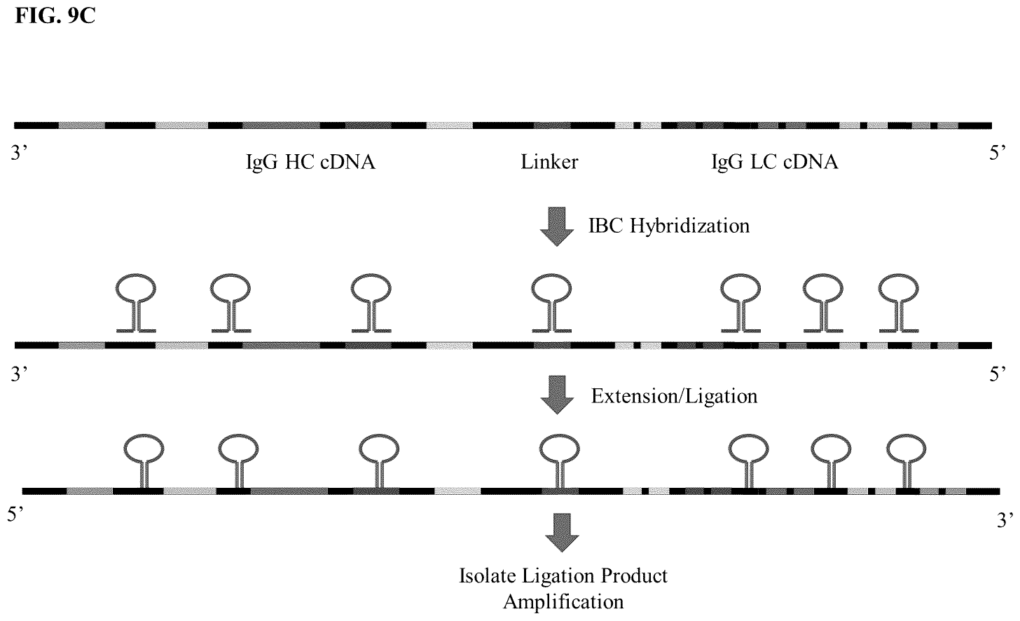

As used herein, the phrase “linking the overlapped polynucleotide complex” refers to the process of generating a single polynucleotide including a sequence of, at least, a first independent polynucleotide and a second independent polynucleotide. For example, using hybridized overlap oligonucleotides (e.g., a first overlap oligonucleotide and a second overlap oligonucleotide, wherein a sequence at the 5′ end of the first overlap oligonucleotide is hybridized to a sequence at the 5′ end of the second overlap oligonucleotide), IgG HC and LC mRNA are annealed to two hybridized overlap oligonucleotides followed by reverse transcription as shown in FIG. 9A, wherein each overlap oligonucleotide is specific for the variable region of the IgG HC or IgG LC mRNA. Reverse transcription generates an overlapped polynucleotide complex include complements of the first and second independent polynucleotides, hybridized together at the 5′ ends of the first and second overlap oligonucleotides. Following reverse transcription, second strand cDNA synthesis is performed (e.g., RNAse H nicking followed by DNA Polymerase I extension and ligation of the products to form a contiguous cDNA strand), thereby linking the overlapped polynucleotide complex, and generating a bridged polynucleotide as described herein.

In embodiments, the bridged polynucleotide includes the full-length sequence of each of the first independent polynucleotide and the second independent polynucleotide. In embodiments, the bridged polynucleotide includes the full-length sequence of the first independent polynucleotide. In embodiments, the bridged polynucleotide includes the full-length sequence of the second independent polynucleotide.

In embodiments, the method further includes hybridizing a 5′ terminal oligonucleotide probe downstream of the one or more interposing oligonucleotide probes to the first polynucleotide, and hybridizing a 3′ terminal oligonucleotide probe upstream of the one or more interposing oligonucleotide probes to the second polynucleotide; wherein the 5′ terminal oligonucleotide probe includes from 5′ to 3′: i. a hybridization sequence complementary to a sequence of the first polynucleotide; and ii. a primer binding sequence; and wherein the 3′ terminal oligonucleotide probe includes from 3′ to 5′: i. a hybridization sequence complementary to a sequence of the second polynucleotide; and ii. a primer binding sequence.

In an aspect is provided a method of amplifying a tagged complement of two independent single-stranded polynucleotides, the method including: i) hybridizing a bridge oligonucleotide to the first independent polynucleotide and the second independent polynucleotide, thereby forming a bridged polynucleotide complex, wherein the bridge oligonucleotide includes from 5′ to 3′ a first hybridization sequence complementary to the first independent polynucleotide, a linking polynucleotide sequence, and a second hybridization sequence complementary to the second independent polynucleotide; ii) amplifying the bridged polynucleotide complex, thereby generating a bridged polynucleotide including a sequence of a first independent polynucleotide linked to a sequence of a second independent polynucleotide or a complement thereof; iii) hybridizing to the bridged polynucleotide a plurality of interposing oligonucleotide barcodes; iv) extending the 3′ ends of the interposing oligonucleotide barcodes with one or more polymerases to create extension products; v) ligating adjacent ends of the extension products hybridized to the bridged polynucleotide thereby making an integrated strand tagged with a plurality of interposing oligonucleotide barcodes, wherein the integrated strand includes sequences of the first and second independent polynucleotides or complements thereof; and vi) amplifying the integrated strand by an amplification reaction; wherein each of the interposing oligonucleotide barcodes includes from 5′ to 3′: a. a first hybridization sequence complementary to a first sequence of the bridged polynucleotide; b. a first stem region including a sequence common to the plurality of interposing oligonucleotide barcodes; c. a loop region including a barcode sequence, wherein the barcode sequence, alone or in combination with a sequence of one or both of (a) the bridged polynucleotide, or (b) one or more additional barcode sequences, distinguishes the bridged polynucleotide from bridged polynucleotides generated from other cells; d. a second stem region including a sequence complementary to the first stem region, wherein the second stem region is capable of hybridizing to the first stem region under hybridization conditions; and e. a second hybridization sequence complementary to a second sequence of the bridged polynucleotide.

In an aspect is provided a method of forming a tagged complement of two independent single-stranded polynucleotides, the method including: i) generating a bridged polynucleotide including a sequence of a first independent polynucleotide of a cell linked to a sequence of a second independent polynucleotide of a cell or a complement thereof, ii) hybridizing one or more of interposing oligonucleotide barcodes to the bridged polynucleotide; iii) extending the 3′ ends of the interposing oligonucleotide barcodes with one or more polymerases to create extension products; iv) ligating adjacent ends of the extension products hybridized to the bridged polynucleotide thereby making an integrated strand tagged with a plurality of interposing oligonucleotide barcodes, wherein the integrated strand includes sequences of the first and second independent polynucleotides or complements thereof. In embodiments, the method includes amplifying the integrated strand by one or more amplification reactions to generate amplification products. In embodiments, the method further includes detecting the integrated strand, the amplification products, and/or the barcodes. In embodiments, one or more of the interposing oligonucleotide barcodes includes from 5′ to 3′: a. a first hybridization sequence complementary to a first sequence of the bridged polynucleotide; b. a first stem region including a sequence common to the plurality of interposing oligonucleotide barcodes; c. a loop region including a barcode sequence, wherein the barcode sequence, alone or in combination with a sequence of one or both of (a) the bridged polynucleotide, or (b) one or more additional barcode sequences, uniquely distinguishes the bridged polynucleotide from bridged polynucleotides generated from other cells; d. a second stem region including a sequence complementary to the first stem region, wherein the second stem region is capable of hybridizing to the first stem region under hybridization conditions; and e. a second hybridization sequence complementary to a second sequence of the bridged polynucleotide. In embodiments, prior to i) the cell is immobilized and/or fixed to a solid support. In embodiments, step i) is performed in a cell (e.g., in situ generation of a bridged polynucleotide).

In an aspect is provided a method of forming an integrated strand complement of a bridged polynucleotide including a plurality of oligonucleotide probes, wherein the bridged polynucleotide includes a complement of two independent single-stranded polynucleotides, the method including: a. hybridizing a bridge oligonucleotide to a first independent polynucleotide and a second independent polynucleotide, thereby forming a bridged polynucleotide complex; b. amplifying the bridged polynucleotide complex, thereby generating a bridged polynucleotide including a sequence of the first independent polynucleotide linked to a sequence of the second independent polynucleotide, or a complement thereof, c. hybridizing one or more interposing oligonucleotide probes to the bridged polynucleotide, hybridizing a 5′ terminal oligonucleotide probe downstream of the one or more interposing oligonucleotide probes, and hybridizing a 3′ terminal oligonucleotide probe upstream of the one or more interposing oligonucleotide probes, wherein each of the interposing oligonucleotide probes includes from 5′ to 3′: i. a first hybridization sequence complementary to a first sequence of the bridged polynucleotide; ii. a loop region including a primer binding sequence; and iii. a second hybridization sequence complementary to a second sequence of the bridged polynucleotide; wherein the 5′ terminal oligonucleotide probe includes from 5′ to 3′: i. a hybridization sequence complementary to a third sequence of the bridged polynucleotide; and ii. a primer binding sequence; and wherein the 3′ terminal oligonucleotide probe includes from 3′ to 5′: i. a hybridization sequence complementary to a fourth sequence of the bridged polynucleotide; and ii. a primer binding sequence; d. extending the 3′ end of each second hybridization sequence of the interposing oligonucleotide probes and the 3′ end of the hybridization sequence of the 3′ terminal oligonucleotide probe with one or more polymerases thereby forming an extension product of each of the oligonucleotide probes; e. ligating the 3′ end of each of the extension products to the 5′ end of the adjacent extension products, and ligating the 5′ end of the 5′ terminal oligonucleotide probe to the 3′ end of the adjacent extension product, each hybridized to the same bridged polynucleotide thereby making an integrated strand including a complement of the bridged polynucleotide including a plurality of the oligonucleotide probes; and f. amplifying the integrated strand by an amplification reaction to produce a complement of the integrated strand thereby forming an integrated strand complement of the bridged polynucleotide including oligonucleotide probes, wherein the complement of the integrated strand includes a complement of the plurality of oligonucleotide probes.

In embodiments, the method further includes extending the 3′ end of the hybridization sequence of the 3′ terminal oligonucleotide probe with one or more polymerases thereby forming an extension product.

In embodiments, the method further includes ligating the 5′ end of the 5′ terminal oligonucleotide probe to the 3′ end of the adjacent extension product. Examples of enzymes useful for ligation include CircLigase™ enzyme, Taq DNA Ligase, HiFi Taq DNA Ligase, T4 DNA ligase, T4 RNA ligase, T4 RNA ligase 2, or Ampligase® DNA Ligase.

In embodiments, the bridge oligonucleotide includes from 5′ to 3′: i. a first hybridization sequence complementary to a 3′ terminal sequence of the first independent polynucleotide; ii. a linker sequence; and iii. a second hybridization sequence complementary to a 5′ terminal sequence of the second independent polynucleotide.

In embodiments, the 5′ end of the bridge oligonucleotide includes about 5 to about 50 nucleotides complementary to the 3′ end of the first independent polynucleotide, and wherein the 3′ end of the bridge oligonucleotide includes about 5 to about 50 nucleotides complementary to the 5′ end of the second independent polynucleotide.

In embodiments, each interposing oligonucleotide barcode includes a first stem region including a sequence common to the plurality of interposing oligonucleotide barcodes and a second stem region including a sequence complementary to the first stem region, wherein the second stem region is capable of hybridizing to the first stem region under hybridization conditions.

In embodiments, the first overlap oligonucleotide includes from 5′ to 3′ a first hybridization sequence complementary to a 5′ sequence of the second overlap oligonucleotide, and a second hybridization sequence complementary to a 3′ sequence of the first independent polynucleotide, and wherein the second overlap oligonucleotide includes from 5′ to 3′ a first hybridization sequence complementary to a 5′ sequence of the first overlap oligonucleotide and a second hybridization sequence complementary to a 5′ sequence of the second independent polynucleotide.

In embodiments, prior to step (a), the method further includes isolating a cell including a plurality of polynucleotides, wherein the plurality of polynucleotides includes the first independent polynucleotide and the second independent polynucleotide. In embodiments, prior to step (a), the method further includes isolating a cell including a plurality of polynucleotides, wherein the plurality of polynucleotides includes the first independent polynucleotide and the second independent polynucleotide.

In embodiments, the loop region includes a sample index sequence. In embodiments, the loop region is a sample index sequence. In embodiments, a tagged complement of a sample polynucleotide refers to a complementary nucleic acid sequence that contains an interposing oligonucleotide barcode as described herein. In embodiments, the tagged complements include at least two interposing oligonucleotide barcodes. In embodiments, the tagged complements include at least three interposing oligonucleotide barcodes. In embodiments, the tagged complements include at least four interposing oligonucleotide barcodes. In embodiments, the tagged complements include at least 5 interposing oligonucleotide barcodes.

In embodiments, the method further includes sequencing the amplified products. In embodiments, the method further includes sequencing the amplified integrated strand. In embodiments, the sequencing includes: (A) fragmenting the amplified products of step (vi) to produce fragments, (B) ligating adapters to the fragments, (C) amplifying the resultant products from step (B) to generate amplicons, and (D) performing a sequencing reaction on the amplicons from step (C). In embodiments, the sequencing further includes (a) producing a plurality of sequencing reads; (b) grouping sequencing reads based on co-occurrence of barcode sequences; and (c) within each group, aligning the reads that belong to the same strand of an original sample polynucleotide based on the sequences of the barcode sequences. In embodiments, the sequencing includes sequencing by synthesis, sequencing by ligation, or pyrosequencing. In embodiments, sequencing includes sequencing by binding. In embodiments, the sequencing includes sequencing by synthesis, sequencing by binding, sequencing by ligation, or pyrosequencing.

In embodiments, the method further includes sequencing the amplified product of step (f). In embodiments, the sequences further includes (a) producing a plurality of sequencing reads; (b) grouping sequencing reads based on co-occurrence of interposing oligonucleotide probe sequences; and (c) within each group, aligning the sequencing reads that belong to the same strand of an original bridged polynucleotide based on the sequences of the interposing oligonucleotide probe sequences. In embodiments, prior to sequencing, the method further includes hybridizing a sequencing primer to the primer binding sequence of one of the plurality of interposing oligonucleotide probes in the integrated strands. In embodiments, prior to sequencing, the method further includes hybridizing a sequencing primer to the primer binding sequence of the integrated oligonucleotide probe.

In an aspect is provided a method of sequencing at least three regions of the integrated strand complement of the bridged polynucleotide includes the oligonucleotide probes any one of the aspects and embodiments described herein, the method including: (a) contacting a first primer annealed to a first region of the integrated strand complement with a sequencing solution including a plurality of nucleotides and incorporating with a polymerase one or more nucleotides into the first primer to create a first extension strand, and detecting the one or more incorporated nucleotides so as to identify each incorporated nucleotide in the first extension strand; (b) contacting the integrated strand complement with a blocking element thereby terminating extension of the first extension strand thereby forming a blocked first extension strand; (c) contacting a second primer annealed to a second region of the integrated strand complement with a sequencing solution including a plurality of nucleotides and incorporating with a polymerase one or more nucleotides into the second primer to create a second extension strand, and detecting the one or more incorporated nucleotides so as to identify each incorporated nucleotide in the second extension strand; (d) contacting the integrated strand complement with a blocking element thereby terminating extension of the second extension strand and creating a blocked second extension strand; and (e) contacting a third primer annealed to a third region of the integrated strand complement with a sequencing solution including a plurality of nucleotides and incorporating with a polymerase one or more nucleotides into the third primer to create a third extension strand, and detecting the one or more incorporated nucleotides so as to identify each incorporated nucleotide in the third extension strand.

In embodiments, the blocked first extension strand is upstream of the blocked second extension strand, third extension strand, or both the blocked second extension strand and third extension strand.

In embodiments, the blocking element includes a chain-terminating nucleotide. In embodiments, the chain-terminating nucleotide includes a ddNTP, a reversibly-terminated dNTP, or a modified nucleotide triphosphate which lacks a 3′-OH.

In embodiments, contacting the integrated strand complement with a blocking element includes hybridizing a blocking oligonucleotide downstream of the extension strand. In embodiments, the blocking oligonucleotide includes locked nucleic acids (LNAs), Bis-locked nucleic acids (bisLNAs), twisted intercalating nucleic acids (TINAs), bridged nucleic acids (BNAs), 2′-O-methyl RNA:DNA chimeric nucleic acids, minor groove binder (MGB) nucleic acids, morpholino nucleic acids, C5-modified pyrimidine nucleic acids, peptide nucleic acids (PNAs), phosphorothioate nucleic acids, or combinations thereof. In embodiments, the blocking oligonucleotide inhibits nucleotide incorporation.

In embodiments, the 3′ end of one or more of the extension strands is capable of ligating to the 5′ end of one or more different extension strands.

In embodiments, the method further includes contacting the integrated strand complement with a blocking element thereby terminating extension of the third extension strand thereby forming a blocked third extension strand. In embodiments, the method further includes contacting a fourth primer annealed to a fourth region of the integrated strand complement and incorporating one or more nucleotides into the fourth primer with a polymerase to create a fourth extension strand, and detecting the one or more incorporated nucleotides so as to identify each incorporated nucleotide in the fourth extension strand.

In embodiments, between 4 to 9 regions or 9 to 15 regions of the integrated strand complement are sequenced. In embodiments, between 15 to 30 regions or 30 to 50 regions of the integrated strand complement are sequenced. In embodiments, 4, 5, 6, 7, 8, 9, 10, 11, 12, 13, 14, or 15 regions of the integrated strand complement are sequenced. In embodiments, 16, 27, 18, 19, 20, 21, 22, 23, 24, 25, 26, 27, 28, 29, or 30 regions of the integrated strand complement are sequenced. In embodiments, 31, 32, 33, 34, 35, 36, 37, 38, 39, 40, 41, 42, 43, 44, 45, 46, 47, 48, 49, 50, or more regions of the integrated strand complement are sequenced.

In embodiments, sequencing is performed according to a “sequencing-by-binding” method (see, e.g., U.S. Pat. Pubs. US2017/0022553 and US2019/0048404, each of which is incorporated herein by reference in its entirety), which refers to a sequencing technique wherein specific binding of a polymerase and cognate nucleotide to a primed template nucleic acid molecule (e.g., blocked primed template nucleic acid molecule) is used for identifying the next correct nucleotide to be incorporated into the primer strand of the primed template nucleic acid molecule. The specific binding interaction need not result in chemical incorporation of the nucleotide into the primer. In some embodiments, the specific binding interaction can precede chemical incorporation of the nucleotide into the primer strand or can precede chemical incorporation of an analogous, next correct nucleotide into the primer. Thus, detection of the next correct nucleotide can take place without incorporation of the next correct nucleotide.

In embodiments, the sequencing further includes (a) producing a plurality of sequencing reads; (b) grouping sequencing reads based on co-occurrence of barcode sequences; and (c) within each group, aligning the reads that belong to the same strand of an original sample polynucleotide based on the sequences of the barcode sequences. In embodiments, each of the sequencing reads include at least a portion of two or more barcode sequences, or complements thereof. In embodiments, aligning the reads includes alignment to a reference genome. In embodiments, the method further includes forming a consensus sequence for reads having the same barcode sequence.

In embodiments, the method further includes computationally reconstructing sequences of a plurality of individual strands of original sample polynucleotides by removing interposing oligonucleotide barcode-derived sequences and joining sequences for adjacent portions of the sample polynucleotide. In embodiments, the method further includes forming a consensus sequence for reads having the same barcode sequence.

In embodiments, generating the bridged polynucleotide includes hybridizing a bridge oligonucleotide to the first independent polynucleotide and the second independent polynucleotide to generate the bridged polynucleotide, wherein the bridge oligonucleotide includes from 5′ to 3′ a first hybridization sequence complementary to the first independent polynucleotide, a linking polynucleotide sequence, and a second hybridization sequence complementary to the second independent polynucleotide. In embodiments, the bridge oligonucleotide includes one or more barcode sequences.

In embodiments, generating the bridged polynucleotide includes overlap-extension PCR (OE-PCR). In embodiments, amplifying the overlapped polynucleotide complex includes overlap-extension PCR (OE-PCR). In embodiments, the OE-PCR includes a reverse transcription step (e.g., wherein one or more hybridized primers are extended using a reverse transcriptase to form cDNA). In embodiments, the OE-PCR utilizes an overlap-extension primer mix. In embodiments, the overlap-extension primer mix includes primer sets wherein at least one primer set member of each primer set includes an overlap-extension tail capable of hybridizing to the overlap-extension tail of a primer set member of a second primer set. In embodiments, OE-PCR utilizes at least two overlap oligonucleotides. In embodiments, two overlap oligonucleotides at hybridized at a 5′ sequence of each of the overlap oligonucleotides. In embodiments, a 3′ sequence of each overlap oligonucleotide is complementary to a 3′ sequence of an independent polynucleotide.

In embodiments, generating the bridged polynucleotide includes hybridizing a bridge oligonucleotide to a 3′ end of the first independent polynucleotide and a 5′ end of the second independent polynucleotide. In embodiments, step a) includes hybridizing a bridge oligonucleotide to a 3′ end of the first independent polynucleotide and a 5′ end of the second independent polynucleotide. In embodiments, generating the bridged polynucleotide includes linking a sequence from a 5′ end of the first independent polynucleotide to a complement of a sequence from a 5′ end of the second independent polynucleotide. In embodiments, step a) includes linking a sequence from a 5′ end of the first independent polynucleotide to a complement of a sequence from a 5′ end of the second independent polynucleotide.

As used herein, the terms “bridge oligonucleotide,” or “bridging oligonucleotide” refer to an oligonucleotide with a first region of complementarity to a first independent polynucleotide at the 5′ end of the bridge oligonucleotide, and a second region of complementarity to a second independent polynucleotide at the 3′ end of the bridge oligonucleotide. In embodiments, the first independent polynucleotide and the second independent polynucleotide are different or substantially different. In embodiments, the first independent polynucleotide and the second independent polynucleotide are the same or substantially the same.

As used herein, the terms “overlap oligonucleotides” or “overlapping oligonucleotides” refer to at least two oligonucleotides, wherein the 3′ end (e.g., the downstream end) of the first overlap oligonucleotide includes a first region of complementarity to a 3′ sequence of a first independent polynucleotide, and wherein the 3′ end of the second overlap oligonucleotide includes a second region of complementarity to a 3′ sequence of a second independent polynucleotide, wherein the 5′ ends (e.g., the upstream ends) of the first and second overlap oligonucleotides are complementary to each other. In embodiments, a first overlap oligonucleotide includes, from 3′ to 5′, a first hybridization sequence complementary to a first independent polynucleotide and a sequence complementary to a second overlap oligonucleotide. In embodiments, a second overlap oligonucleotide includes, from 3′ to 5′, a second hybridization sequence complementary to a second independent polynucleotide and a sequence complementary to the first overlap oligonucleotide. In embodiments, the first hybridization sequence is complementary to a 3′ sequence of the first independent polynucleotide. In embodiments, the second hybridization sequence is complementary to a 3′ sequence of the second independent polynucleotide.

In embodiments, the 5′ end of the bridge oligonucleotide includes about 5 to about 50 nucleotides complementary to the 3′ end of the first independent polynucleotide. In embodiments, the 5′ end of the bridge oligonucleotide includes about 15 to about 40 nucleotides complementary to the 3′ end of the first independent polynucleotide. In embodiments, the 5′ end of the bridge oligonucleotide includes about 5, 10, 15, 20, 25, 30, 35, 40, 45, 50, or more nucleotides complementary to the 3′ end of the first independent polynucleotide.

In embodiments, the 3′ end of the bridge oligonucleotide includes about 5 to about 50 nucleotides complementary to the 5′ end of the first independent polynucleotide. In embodiments, the 3′ end of the bridge oligonucleotide includes about 15 to about 40 nucleotides complementary to the 5′ end of the first independent polynucleotide. In embodiments, the 3′ end of the bridge oligonucleotide includes about 5, 10, 15, 20, 25, 30, 35, 40, 45, 50, or more nucleotides complementary to the 5′ end of the first independent polynucleotide.

In embodiments, the 5′ end of the first overlap oligonucleotide includes about 5 to about 50 nucleotides complementary to the 5′ end of the second overlap oligonucleotide. In embodiments, the 5′ end of the first overlap oligonucleotide includes about 15 to about 40 nucleotides complementary to the 5′ end of the second overlap oligonucleotide. In embodiments, the 5′ end of the first overlap oligonucleotide includes about 5, 10, 15, 20, 25, 30, 35, 40, 45, 50, or more nucleotides complementary to the 5′ end of the second overlap oligonucleotide.

In embodiments, the 3′ end of the first overlap oligonucleotide includes about 5 to about 50 nucleotides complementary to the 3′ end of the first independent polynucleotide. In embodiments, the 3′ end of the first overlap oligonucleotide includes about 15 to about 40 nucleotides complementary to the 3′ end of the first independent polynucleotide. In embodiments, the 3′ end of the first overlap oligonucleotide includes about 5, 10, 15, 20, 25, 30, 35, 40, 45, 50, or more nucleotides complementary to the 3′ end of the first independent polynucleotide.

In embodiments, the 3′ end of the second overlap oligonucleotide includes about 5 to about 50 nucleotides complementary to the 3′ end of the second independent polynucleotide. In embodiments, the 3′ end of the second overlap oligonucleotide includes about 15 to about 40 nucleotides complementary to the 3′ end of the second independent polynucleotide. In embodiments, the 3′ end of the second overlap oligonucleotide includes about 5, 10, 15, 20, 25, 30, 35, 40, 45, 50, or more nucleotides complementary to the 3′ end of the second independent polynucleotide.

In embodiments, the bridge oligonucleotide is about 20 to about 250 nucleotides in length. In embodiments, the bridge oligonucleotide is about 20 to 200 nucleotides, 30 to 175 nucleotides, 40 to 150 nucleotides, 50 to 125 nucleotides, or 75 to 100 nucleotides in length. In embodiments, the bridge oligonucleotide is about 20, 30, 40, 50, 75, 100, 125, 150, 175, 200, 225, 250, or more nucleotides in length.

In embodiments, the overlap oligonucleotide is about 20 to about 250 nucleotides in length. In embodiments, the overlap oligonucleotide is about 20 to 200 nucleotides, 30 to 175 nucleotides, 40 to 150 nucleotides, 50 to 125 nucleotides, or 75 to 100 nucleotides in length. In embodiments, the overlap oligonucleotide is about 20, 30, 40, 50, 75, 100, 125, 150, 175, 200, 225, 250, or more nucleotides in length.

In embodiments, the bridge oligonucleotide has an extendable 3′ end. In embodiments, the bridge oligonucleotide is extended by one or more polymerases. In embodiments, the bridge oligonucleotide is extended by a DNA polymerase, or mutant thereof. In embodiments, the bridge oligonucleotide is extended by an RNA polymerase, or mutant thereof. In embodiments, the bridge oligonucleotide is extended by a reverse transcriptase, or mutant thereof.

In embodiments, the overlap oligonucleotide has an extendable 3′ end. In embodiments, the overlap oligonucleotide is extended by one or more polymerases. In embodiments, the overlap oligonucleotide is extended by a DNA polymerase, or mutant thereof. In embodiments, the overlap oligonucleotide is extended by an RNA polymerase, or mutant thereof. In embodiments, the overlap oligonucleotide is extended by a reverse transcriptase, or mutant thereof.

In embodiments, the 5′ terminal oligonucleotide probe includes from 5′ to 3′: i. a first hybridization sequence complementary to a first 5′ terminal sequence of the bridged polynucleotide; ii. a loop region including a primer binding sequence; and iii. a second hybridization sequence complementary to a second 5′ terminal sequence of the bridged polynucleotide, wherein the first and second 5′ terminal sequences are upstream of the bridged polynucleotide sequence complementary to the interposing oligonucleotide probes.

In embodiments, the 3′ terminal oligonucleotide probe includes from 3′ to 5′: i. a first hybridization sequence complementary to a first 3′ terminal sequence of the bridged polynucleotide; ii. a loop region including a primer binding sequence; and iii. a second hybridization sequence complementary to a second 3′ terminal sequence of the bridged polynucleotide, wherein the first and second 3′ terminal sequences are downstream of the bridged polynucleotide sequence complementary to the interposing oligonucleotide probes.

In embodiments, the first hybridization sequence, the second hybridization sequence, and the primer binding sequence is different between each interposing oligonucleotide probe of a plurality of interposing oligonucleotide probe.

In embodiments, step a) includes hybridizing a bridge oligonucleotide to a 3′ end of the first independent polynucleotide and a 5′ end of the second independent polynucleotide. In embodiments, step a) includes linking a sequence from a 5′ end of the first independent polynucleotide to a complement of a sequence from a 5′ end of the second independent polynucleotide.

In embodiments, each of the interposing oligonucleotide barcodes include a phosphorylated 5′ end. In embodiments, the method includes phosphorylating the 5′ ends of the interposing oligonucleotide barcodes prior to step (v). Phosphorylating the 5′ ends may be achieved using known techniques in the art (e.g., incubation with a phosphorylating enzyme such as a T4 polynucleotide kinase (PNK) under suitable phosphorylating conditions)

In embodiments, each hybridization sequence includes about 9 to about 15 nucleotides. In embodiments, each hybridization sequence includes about 8 to about 12 nucleotides. In embodiments, each hybridization sequence includes a targeted primer sequence. In embodiments, each hybridization sequence includes at least one locked nucleic acid.

In embodiments, the total combined length of the first hybridization sequence and the second hybridization sequence includes about 18 to about 25 nucleotides. In embodiments, the first and second stem regions are complementary, and each stem region includes a known sequence of about 5 to about 10 nucleotides. In embodiments, the first and second stem regions are complementary, and each stem region includes a known sequence of about 6 to about 8 nucleotides.

In embodiments, the loop region includes about 5 to about 20 nucleotides, or about 10 to about 20 nucleotides. In embodiments, the loop region includes about 12 to about 16 nucleotides.

In embodiments, each barcode sequence is selected from a set of barcode sequences represented by a random or partially random sequence. In embodiments, each barcode sequence is selected from a set of barcode sequences represented by a random sequence. In embodiments, the loop region further includes a sample index sequence. In embodiments, each barcode sequence differs from every other barcode sequence by at least two nucleotide positions. In embodiments, each barcode in a plurality of barcodes differs from every other barcode in the plurality by at least three nucleotide positions, such as at least 3, 4, 5, 6, 7, 8, 9, 10, or more nucleotide positions. In embodiments, the barcodes are selected to form a known set of barcodes, e.g., the set of barcodes may be distinguished by a particular Hamming distance.

In embodiments, the barcodes in the known set of barcodes have a specified Hamming distance. In embodiments, the Hamming distance is 4 to 15. In embodiments, the Hamming distance is 8 to 12. In embodiments, the Hamming distance is 10. In embodiments, the Hamming distance is 0 to 100. In embodiments, the Hamming distance is 0 to 15. In embodiments, the Hamming distance is 0 to 10. In embodiments, the Hamming distance is 1 to 10. In embodiments, the Hamming distance is 5 to 10. In embodiments, the Hamming distance is 1 to 100. In embodiments, the Hamming distance between any two barcode sequences of the set is at least 2, 3, 4, or 5. In embodiments, the Hamming distance between any two barcode sequences of the set is at least 3. In embodiments, the Hamming distance between any two barcode sequences of the set is at least 4.

In embodiments, each of the two independent single-stranded polynucleotides include a gene or gene fragment. In embodiments, the gene or gene fragment is a cancer-associated gene or fragment thereof, T cell receptor (TCRs) gene or fragment thereof, or a B cell receptor (BCRs) gene, or fragment thereof. In embodiments, the gene or gene fragment is a CDR3 gene or fragment thereof, T cell receptor alpha variable (TRAV) gene or fragment thereof, T cell receptor alpha joining (TRAJ) gene or fragment thereof, T cell receptor alpha constant (TRAC) gene or fragment thereof, T cell receptor beta variable (TRBV) gene or fragment thereof, T cell receptor beta diversity (TRBD) gene or fragment thereof, T cell receptor beta joining (TRBJ) gene or fragment thereof, T cell receptor beta constant (TRBC) gene or fragment thereof, T cell receptor gamma variable (TRGV) gene or fragment thereof, T cell receptor gamma joining (TRGJ) gene or fragment thereof, T cell receptor gamma constant (TRGC) gene or fragment thereof, T cell receptor delta variable (TRDV) gene or fragment thereof, T cell receptor delta diversity (TRDD) gene or fragment thereof, T cell receptor delta joining (TRDJ) gene or fragment thereof, or T cell receptor delta constant (TRDC) gene or fragment thereof.

In embodiments, each of the two independent single-stranded polynucleotides include messenger RNA (mRNA). In embodiments, each of the two independent single-stranded polynucleotides include messenger RNA (mRNA), tRNA (transfer RNA), rRNA (ribosomal RNA), and/or noncoding RNA (such as lncRNA (long noncoding RNA)). In embodiments, the two single-stranded polynucleotides are considered to be independent (e.g., not attached to each other) prior to employing the methods as described herein.

In embodiments, the method further includes hybridizing to the bridged polynucleotide a terminal adapter, wherein the terminal adapter includes a first hybridization sequence complementary to a first sequence of the bridged polynucleotide, a barcode sequence, and a primer binding sequence. In embodiments, a terminal adapter includes at least one hybridization sequence as described herein (e.g., a hybridization sequence of about 10 to about 30 nucleotides in length), a barcode (e.g., a UMI of about 8 to about 15 nucleotides in length), and a primer binding site (e.g., an amplification primer binding site of about 10 to about 25 nucleotides in length). In embodiments, the terminal adapter does not include a loop region or a stem region (e.g., a loop region or stem region as described herein). In embodiments, the terminal adapter is a single-stranded polynucleotide having at least one primer binding sequence. In embodiments, the terminal adapter includes at least one amplification primer binding sequence. In embodiments, the terminal adapter includes two or more amplification primer binding sequences. The amplification primer binding sequence refers to a nucleotide sequence that is complementary to a primer useful in initiating amplification (i.e., an amplification primer). Primer binding sequences usually have a length in the range of between 3 to 36 nucleotides, also 5 to 24 nucleotides, also from 14 to 36 nucleotides. In embodiments, the terminal adapter includes a barcode of about 8 nucleotides. In embodiments, the terminal adapter includes a barcode of about 12 nucleotides. In embodiments, the terminal adapter includes a barcode of about 15 nucleotides. In embodiments, the first and second hybridization sequences have a total length of 15 to 25 nucleotides. In embodiments, the method includes hybridizing two terminal adapters to the sample polynucleotide.

In embodiments, the method further includes hybridizing a first terminal adapter having the sequence from 5′ to 3′, a primer binding sequence, a barcode, a first hybridization sequence and a second hybridization sequence to 3′ end of a sample polynucleotide. In embodiments, the method further includes hybridizing a second terminal adapter having the sequence from 5′ to 3′, a first hybridization sequence and a second hybridization sequence, an index, and a primer binding sequence, wherein the first and the second hybridization sequences anneal to the 5′ end of a sample polynucleotide. In embodiments, both first and second terminal adapters are hybridized to a sample polynucleotide. In embodiments, amplifying includes hybridizing an amplification primer to the primer binding sequence of the terminal adapter and cycles of primer extension with a polymerase and nucleotides to generate amplified products.

In embodiments, the terminal adapter includes one or more phosphorothioate containing nucleotides. For example, one terminal adapter may include five terminal phosphorothioate linkages on the 3′ end to prevent exonuclease degradation (e.g., exonuclease degradation by T4 DNA Polymerase). In embodiments, the terminal adapter includes one or more LNAs. In embodiments, the terminal adapter includes a modified nucleotide that contains an affinity tag (e.g., a biotin-containing nucleotide). The biotin-containing terminal adapter, for example, could then facilitate affinity purification of the tagged complement.

In embodiments, the methods of making tagged complements of a plurality of sample polynucleotides include extending the 3′ ends of the interposing oligonucleotide barcodes with one or more polymerases to create extension products. Methods of extending 3′ ends of oligonucleotides are known to those skilled in the art. In embodiments, extension is achieved by a DNA polymerase without strand displacement activity.

In embodiments, the methods of making tagged complements of a plurality of sample polynucleotides include ligating adjacent ends of extension products hybridized to the same sample polynucleotide thereby making complements of the plurality of sample polynucleotides tagged with a plurality of interposing oligonucleotide barcodes. Methods of ligation are known to those skilled in the art. In embodiments, the ligation includes enzymatic ligation. In embodiments, ligating includes enzymatic ligation including a ligation enzyme (e.g., CircLigase™ enzyme, Taq DNA Ligase, HiFi Taq DNA Ligase, T4 ligase, PBCV-1 DNA Ligase (also known as SplintR ligase) or Ampligase DNA Ligase). Non-limiting examples of ligases include DNA ligases such as DNA Ligase I, DNA Ligase II, DNA Ligase III, DNA Ligase IV, T4 DNA ligase, T7 DNA ligase, T3 DNA Ligase, E. coli DNA Ligase, PBCV-1 DNA Ligase (also known as SplintR ligase) or a Taq DNA Ligase. In embodiments, the ligating enzyme is T4 RNA ligase, T4 DNA ligase, T4 RNA ligase 2, Taq DNA ligase, or E. coli DNA ligase.

In embodiments, ligating includes chemical ligation (e.g., enzyme-free, click-mediated ligation). In embodiments, the extension products include a first bioconjugate reactive moiety capable of bonding upon contact with a second (complementary) bioconjugate reactive moiety. In embodiments, the extension products include an alkynyl moiety at the 3′ and an azide moiety at the 5′ end that, upon hybridization to the target nucleic acid react to form a triazole linkage during suitable reaction conditions. Reaction conditions and protocols for chemical ligation techniques that are compatible with nucleic acid amplification methods are known in the art, for example El-Sagheer, A. H., & Brown, T. (2012). Accounts of chemical research, 45(8), 1258-1267; Manuguerra I. et al. Chem Commun (Camb). 2018; 54(36):4529-4532; and Odeh, F., et al. (2019). Molecules (Basel, Switzerland), 25(1), 3, each of which is incorporated herein by reference in their entirety.

In embodiments, the methods of making tagged complements provided herein include interposing oligonucleotide barcodes according to any of the aspects disclosed herein. In embodiments, the methods of making tagged complements described herein include interposing oligonucleotide barcodes that include a phosphorylated 5′ end.

In embodiments, the methods of making tagged complements provided herein do not include interposing oligonucleotide barcodes with a phosphorylated 5′ end. In embodiments, the method includes phosphorylating the 5′ ends of the interposing barcodes prior to step (c). Phosphorylation may be performed, before, during, or after extension. In embodiments, phosphorylation occurs in parallel with the extension reaction. In embodiments, ligation reaction occurs in parallel with the extension reaction.

In embodiments, the methods of making tagged complements provided herein further include sequencing the tagged complements. For example, sequencing the tagged complements provides sequence information about the two single-stranded polynucleotides.

In embodiments, the methods of making tagged complements provided herein include sequencing, where sequencing includes (a) amplifying the tagged complements of the plurality of sample polynucleotides by an amplification reaction thereby making amplified products; and (b) performing a sequencing reaction on the amplified products.

The nucleic acids described herein (e.g., the integrated strand, or the tagged complements) can be amplified by a suitable method. The term “amplified” as used herein refers to subjecting a target nucleic acid in a sample to a process that linearly or exponentially generates amplicon nucleic acids having the same or substantially the same (e.g., substantially identical) nucleotide sequence as the target nucleic acid, or segment thereof, and/or a complement thereof. In some embodiments an amplification reaction includes a suitable thermal stable polymerase. Thermal stable polymerases are known in the art and are stable for prolonged periods of time, at temperature greater than 80° C. when compared to common polymerases found in most mammals. In certain embodiments the term “amplified” refers to a method that includes a polymerase chain reaction (PCR). Conditions conducive to amplification (i.e., amplification conditions) are known and often include at least a suitable polymerase, a suitable template, a suitable primer or set of primers, suitable nucleotides (e.g., dNTPs), a suitable buffer, and application of suitable annealing, hybridization and/or extension times and temperatures. In certain embodiments an amplified product (e.g., an amplicon) can contain one or more additional and/or different nucleotides than the template sequence, or portion thereof, from which the amplicon was generated (e.g., a primer can contain “extra” nucleotides (such as a 5′ portion that does not hybridize to the template), or one or more mismatched bases within a hybridizing portion of the primer).

A nucleic acid can be amplified by a thermocycling method or by an isothermal amplification method. In some embodiments, a rolling circle amplification method is used. In some embodiments, amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support.

In some embodiments solid phase amplification includes a nucleic acid amplification reaction including only one species of oligonucleotide primer immobilized to a surface or substrate. In certain embodiments solid phase amplification includes a plurality of different immobilized oligonucleotide primer species. In some embodiments solid phase amplification may include a nucleic acid amplification reaction including one species of oligonucleotide primer immobilized on a solid surface and a second different oligonucleotide primer species in solution. Multiple different species of immobilized or solution-based primers can be used. Non-limiting examples of solid phase nucleic acid amplification reactions include interfacial amplification, bridge amplification, emulsion PCR, WildFire amplification (e.g., U.S. Patent Publ. No. 2013/0012399), the like or combinations thereof.

In embodiments, amplifying includes hybridizing an amplification primer to the primer binding sequence of the terminal adapter and cycles of primer extension with a polymerase and nucleotides to generate amplified products. In embodiments, the amplification reaction includes polymerase chain reaction (PCR), strand displacement amplification (SDA), multiple displacement amplification (MDA), ligation chain reaction, transcription mediated amplification (TMA), nucleic acid sequence-based amplification (NASBA), rolling circle amplification (RCA), exponential rolling circle amplification (eRCA), hyperbranched rolling circle amplification (HRCA), or a combination thereof. Suitable methods for amplification include, but are not limited to, the polymerase chain reaction (PCR), strand displacement amplification (SDA), transcription mediated amplification (TMA) and nucleic acid sequence-based amplification (NASBA), for example, as described in U.S. Pat. No. 8,003,354, which is incorporated herein by reference in its entirety. The above amplification methods can be employed to amplify one or more nucleic acids of interest. For example, PCR, multiplex PCR, SDA, TMA, NASBA and the like can be utilized to amplify immobilized nucleic acid fragments. In embodiments, amplification includes thermal bridge polymerase chain reaction amplification; for example, as exemplified by the disclosures of U.S. Pat. Nos. 5,641,658; 7,115,400; 7,790,418; U.S. Patent Publ. No. 2008/0009420, each of which is incorporated herein by reference in its entirety. In general, bridge amplification uses repeated steps of annealing of primers to templates, primer extension, and separation of extended primers from templates. Because the forward and reverse primers are attached to the solid substrate, the extension products released upon separation from an initial template are also attached to the solid support. Both strands are immobilized on the solid substrate at the 5′ end, preferably via a covalent attachment. The 3′ end of an amplification product is then permitted to anneal to a nearby reverse primer, forming a “bridge” structure. The reverse primer is then extended to produce a further template molecule that can form another bridge. During bridge PCR, additional chemical additives may be included in the reaction mixture, in which the DNA strands are denatured by flowing a denaturant over the DNA, which chemically denatures complementary strands. This is followed by washing out the denaturant and reintroducing an amplification polymerase in buffer conditions that allow primer annealing and extension.

In embodiments, the amplifying includes rolling circle amplification (RCA) or rolling circle transcription (RCT) (see, e.g., Lizardi et al., Nat. Genet. 19:225-232 (1998), which is incorporated herein by reference in its entirety). Several suitable rolling circle amplification methods are known in the art. For example, RCA amplifies a circular polynucleotide (e.g., DNA) by polymerase extension of an amplification primer complementary to a portion of the template polynucleotide. This process generates copies of the circular polynucleotide template such that multiple complements of the template sequence arranged end to end in tandem are generated (i.e., a concatemer) locally preserved at the site of the circle formation. In embodiments, the amplifying occurs at isothermal conditions. In embodiments, amplifying includes exponential rolling circle amplification (eRCA). Exponential RCA is similar to the linear process except that it uses a second primer having a sequence that is identical to at least a portion of the circular template (Lizardi et al. Nat. Genet. 19:225 (1998)). This two-primer system achieves isothermal, exponential amplification. Exponential RCA has been applied to the amplification of non-circular DNA through the use of a linear probe that binds at both of its ends to contiguous regions of a target DNA followed by circularization using DNA ligase (Nilsson et al. Science 265(5181):208 5(1994)). In embodiments, the amplifying includes hybridization chain reaction (HCR). HCR uses a pair of complementary, kinetically trapped hairpin oligomers to propagate a chain reaction of hybridization events, as described in Dirks, R. M., & Pierce, N. A. (2004) PNAS USA, 101(43), 15275-15278, which is incorporated herein by reference for all purposes. In embodiments, the amplifying includes branched rolling circle amplification (BRCA); e.g., as described in Fan T, Mao Y, Sun Q, et al. Cancer Sci. 2018; 109:2897-2906, which is incorporated herein by reference in its entirety. In embodiments, the amplifying includes hyberbranched rolling circle amplification (HRCA). Hyperbranched RCA uses a second primer complementary to the first amplification product. This allows products to be replicated by a strand-displacement mechanism, which yields drastic amplification within an isothermal reaction (Lage et al., Genome Research 13:294-307 (2003), which is incorporated herein by reference in its entirety). In embodiments, amplifying includes polymerase extension of an amplification primer. In embodiments, the polymerase is T4, T7, Sequenase, Taq, Klenow, and Pol I DNA polymerases. SD polymerase, Bst large fragment polymerase, or a phi29 polymerase or mutant thereof. In embodiments, the polymerase is a strand-displacing polymerase. In embodiments, the strand-displacing polymerase is phi29 polymerase, phi29 mutant polymerase or a thermostable phi29 mutant polymerase. A “phi polymerase” (or “(D29 polymerase”) is a DNA polymerase from the (29 phage or from one of the related phages that, like (D29, contain a terminal protein used in the initiation of DNA replication. For example, phi29 polymerases include the B103, GA-1, PZA, Φ15, BS32, M2Y (also known as M2), Nf, G1, Cp-1, PRD1, PZE, SFS, Cp-5, Cp-7, PR4, PR5, PR722, L17, Φ21, and AV-1 DNA polymerases, as well as chimeras thereof. In embodiments, the polymerase is a phage or bacterial RNA polymerases (RNAPs). In embodiments, the polymerase is a T7 RNA polymerase. In embodiments, the polymerase is an RNA polymerase. Useful RNA polymerases include, but are not limited to, viral RNA polymerases such as T7 RNA polymerase, T3 polymerase, SP6 polymerase, and K11 polymerase; Eukaryotic RNA polymerases such as RNA polymerase I, RNA polymerase II, RNA polymerase III, RNA polymerase IV, and RNA polymerase V; and Archaea RNA polymerase.

In embodiments, amplifying includes extending an amplification primer with a strand-displacing polymerase at a temperature of about 20° C. to about 50° C. In embodiments, the method includes amplifying a template polynucleotide by extending an amplification primer with a strand-displacing polymerase at a temperature of about 30° C. to about 50° C. In embodiments, the method includes amplifying a template polynucleotide by extending an amplification primer with a strand-displacing polymerase at a temperature of about 25° C. to about 45° C. In embodiments, the method includes amplifying a template polynucleotide by extending an amplification primer with a strand-displacing polymerase at a temperature of about 35° C. to about 45° C. In embodiments, the method includes amplifying a template polynucleotide by extending an amplification primer with a strand-displacing polymerase at a temperature of about 35° C. to about 42° C. In embodiments, the method includes amplifying a template polynucleotide by extending an amplification primer with a strand-displacing polymerase at a temperature of about 37° C. to about 40° C. In embodiments, the strand-displacing enzyme is an SD polymerase, Bst large fragment polymerase, or a phi29 polymerase or mutant thereof. In embodiments, the strand-displacing polymerase is phi29 polymerase, phi29 mutant polymerase or a thermostable phi29 mutant polymerase. In embodiments, amplifying includes a plurality of cycles of strand denaturation, primer hybridization, and primer extension.

In embodiments, the methods provided herein include sequencing that includes (a) amplifying the tagged complements of the plurality of sample polynucleotides thereby making amplified products; (b) fragmenting the amplified products to produce fragments, (c) ligating adapters to the fragments, (d) amplifying the resultant products from step (c) to generate a polynucleotide, and (e) performing a sequencing reaction on the polynucleotide from step (d). In embodiments, the amplification method in step (a) is different than the amplification method in step (d). For example, the amplification method in step (a) includes solution phase amplification and the amplification method in step (d) includes solid phase amplification. In embodiments, the adapters have a length of 10 to 50 nucleotides. For example, an adapter may have a length of 10 to 40, 10 to 30, 10 to 20, 25 to 50, 15 to 40, 15 to 30, 20 to 50, 20 to 40, or 20 to 30 nucleotides. In some embodiments, the adapter has a length of 18 to 24 nucleotides. Examples of adapters include, but are not limited to, S1, S2, P5, P7, PE1, PE2, A19, or others known in the art and as provided in commercial kits.

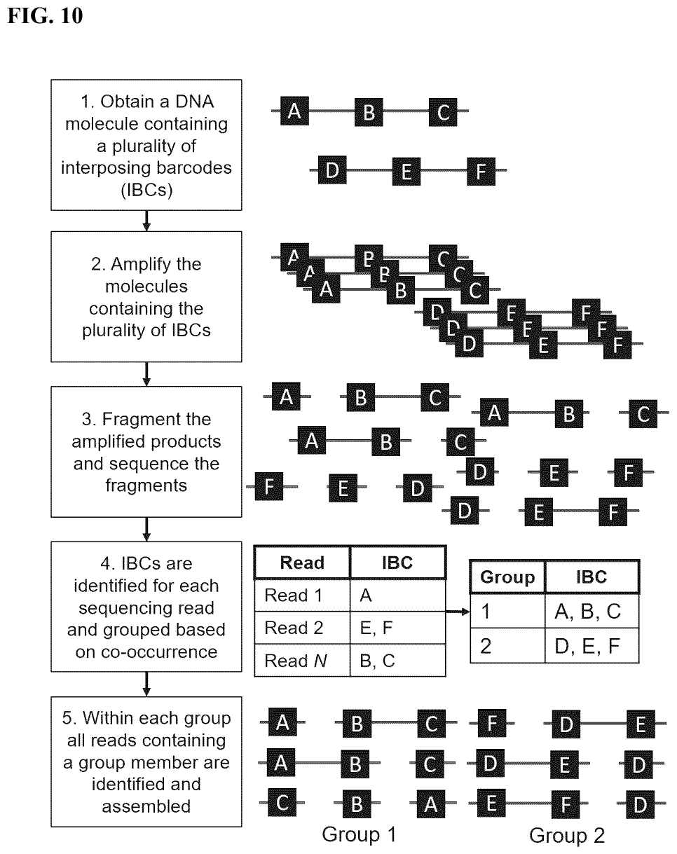

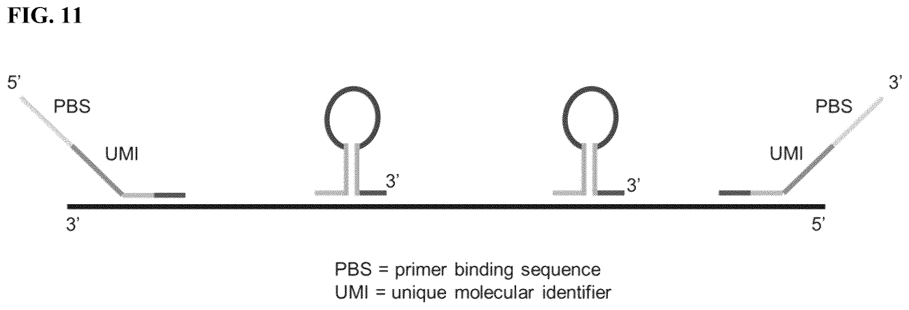

In embodiments, sequencing includes: (a) fragmenting the amplified products to produce fragments, (b) ligating adapters to the fragments, (c) amplifying the resultant products from step (b) to generate a polynucleotide, and (d) performing a sequencing reaction on the polynucleotide from step (c). In embodiments, the sequencing reaction includes (i) immobilizing a polynucleotide to be sequenced on a solid support; (ii) hybridizing a sequencing primer to the immobilized polynucleotide; (iii) performing cycles of primer extension with a polymerase and labeled nucleotides to generate an extended sequencing primer and (iv) detecting the labeled nucleotides to determine the sequence of the immobilized polynucleotide. In embodiments, sequencing further includes (a) producing a plurality of sequencing reads; (b) grouping sequencing reads based on co-occurrence of barcode sequences; and (c) within each group, aligning the reads that belong to the same strand of an original sample polynucleotide based on the sequences of the barcode sequences (see for example FIG. 10).

In embodiments, the methods provided herein include sequencing that includes a sequencing reaction. The sequencing reaction includes (i) immobilizing a polynucleotide to be sequenced on a solid support; (ii) hybridizing a sequencing primer to the immobilized polynucleotide; (iii) performing cycles of primer extension with a polymerase (e.g., a sequencing polymerase) and labeled nucleotides to generate an extended sequencing primer; and (iv) detecting the labeled nucleotides to determine the sequence of the immobilized polynucleotide. In embodiments, the sequencing polymerase is a Taq polymerase, Therminator γ, 9° N polymerase (exo-), Therminator II, Therminator III, or Therminator IX. In embodiments, the sequencing polymerase is Therminator γ. In embodiments, the sequencing polymerase is 9° N polymerase (exo-). In embodiments, the sequencing polymerase is Therminator II. In embodiments, the sequencing polymerase is Therminator III. In embodiments, the sequencing polymerase is Therminator IX. In embodiments, the sequencing polymerase is a Taq polymerase. In embodiments, the sequencing polymerase is a sequencing polymerase. In embodiments, the sequencing polymerase is 9° N and mutants thereof. In embodiments, the sequencing polymerase is Phi29 and mutants thereof. In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044, both of which are incorporated by reference herein). In embodiments, the polymerase is DNA polymerase, a terminal deoxynucleotidyl transferase, or a reverse transcriptase. In embodiments, the enzyme is a DNA polymerase, such as DNA polymerase 812 (Pol 812) or DNA polymerase 1901 (Pol 1901), e.g., a polymerase described in US 2020/0131484, and US 2020/0181587, both of which are incorporated by reference herein.

In embodiments, the sequencing polymerase is a bacterial DNA polymerase, eukaryotic DNA polymerase, archaeal DNA polymerase, viral DNA polymerase, or phage DNA polymerases. Bacterial DNA polymerases include E. coli DNA polymerases I, II and III, IV and V, the Klenow fragment of E. coli DNA polymerase, Clostridium stercorarium (Cst) DNA polymerase, Clostridium thermocellum (Cth) DNA polymerase and Sulfolobus solfataricus (Sso) DNA polymerase. Eukaryotic DNA polymerases include DNA polymerases α, β, γ, δ, €, η, ζ, λ, σ, μ, and k, as well as the Revl polymerase (terminal deoxycytidyl transferase) and terminal deoxynucleotidyl transferase (TdT). Viral DNA polymerases include T4 DNA polymerase, phi-29 DNA polymerase, GA-1, phi-29-like DNA polymerases, PZA DNA polymerase, phi-15 DNA polymerase, Cpl DNA polymerase, Cpl DNA polymerase, T7 DNA polymerase, and T4 polymerase. Other useful DNA polymerases include thermostable and/or thermophilic DNA polymerases such as Thermus aquaticus (Taq) DNA polymerase, Thermus filiformis (Tfi) DNA polymerase, Thermococcus zilligi (Tzi) DNA polymerase, Thermus thermophilus (Tth) DNA polymerase, Thermus flavusu (Tfl) DNA polymerase, Pyrococcus woesei (Pwo) DNA polymerase, Pyrococcus furiosus (Pfu) DNA polymerase and Turbo Pfu DNA polymerase, Thermococcus litoralis (Tli) DNA polymerase, Pyrococcus sp. GB-D polymerase, Thermotoga maritima (Tma) DNA polymerase, Bacillus stearothermophilus (Bst) DNA polymerase, Pyrococcus Kodakaraensis (KOD) DNA polymerase, Pfx DNA polymerase, Thermococcus sp. JDF-3 (JDF-3) DNA polymerase, Thermococcus gorgonarius (Tgo) DNA polymerase, Thermococcus acidophilium DNA polymerase; Sulfolobus acidocaldarius DNA polymerase; Thermococcus sp. go N-7 DNA polymerase; Pyrodictium occultum DNA polymerase; Methanococcus voltae DNA polymerase; Methanococcus thermoautotrophicum DNA polymerase; Methanococcus jannaschii DNA polymerase; Desulfurococcus strain TOK DNA polymerase (D. Tok Pol); Pyrococcus abyssi DNA polymerase; Pyrococcus horikoshii DNA polymerase; Pyrococcus islandicum DNA polymerase; Thermococcus fumicolans DNA polymerase; Aeropyrum pernix DNA polymerase; and the heterodimeric DNA polymerase DP1/DP2. In embodiments, the polymerase is 3PDX polymerase as disclosed in U.S. Pat. No. 8,703,461, the disclosure of which is incorporated herein by reference. In embodiments, the polymerase is a reverse transcriptase. Exemplary reverse transcriptases include, but are not limited to, HIV-1 reverse transcriptase from human immunodeficiency virus type 1 (PDB 1HMV), HIV-2 reverse transcriptase from human immunodeficiency virus type 2, M-MLV reverse transcriptase from the Moloney murine leukemia virus, AMV reverse transcriptase from the avian myeloblastosis virus, or Telomerase reverse transcriptase.

A variety of sequencing methodologies can be used such as sequencing-by synthesis (SBS), pyrosequencing, sequencing by ligation (SBL), or sequencing by hybridization (SBH). In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. A plurality of different nucleic acid fragments that have been attached at different locations of an array can be subjected to an SBS technique under conditions where events occurring for different templates can be distinguished due to their location in the array. In embodiments, the sequencing step includes annealing and extending a sequencing primer to incorporate a detectable label that indicates the identity of a nucleotide in the target polynucleotide, detecting the detectable label, and repeating the extending and detecting of steps. In embodiments, the methods include sequencing one or more bases of a target polynucleotide by extending a sequencing primer hybridized to a target polynucleotide. In embodiments, the sequencing step may be accomplished by a sequencing-by-synthesis (SBS) process. In embodiments, sequencing includes a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 10,738,072, 7,541,444 and 7,057,026. Once such a modified nucleotide has been incorporated into the growing polynucleotide chain complementary to the region of the template being sequenced, there is no free 3′-OH group available to direct further sequence extension and therefore the polymerase cannot add further nucleotides. Once the identity of the base incorporated into the growing chain has been determined, the 3′ block may be removed to allow addition of the next successive nucleotide. By ordering the products derived using these modified nucleotides it is possible to deduce the DNA sequence of the DNA template. Sequencing can be carried out using any suitable sequencing-by-synthesis (SBS) technique, wherein modified nucleotides are added successively to a free 3′ hydroxyl group, typically initially provided by a sequencing primer, resulting in synthesis of a polynucleotide chain in the 5′ to 3′ direction. In embodiments, sequencing includes detecting a sequence of signals. In embodiments, sequencing includes extension of a sequencing primer with labeled nucleotides. Examples of sequencing include, but are not limited to, sequencing by synthesis (SBS) processes in which reversibly terminated nucleotides carrying fluorescent dyes are incorporated into a growing strand, complementary to the target strand being sequenced. In embodiments, the nucleotides are labeled with up to four unique fluorescent dyes. In embodiments, the nucleotides are labeled with at least two unique fluorescent dyes. In embodiments, the readout is accomplished by epifluorescence imaging.

In embodiments, generating a first sequencing read or a second sequencing read includes sequencing-by-binding (see, e.g., U.S. Pat. Pubs. US2017/0022553 and US2019/0048404, each of which is incorporated herein by reference in its entirety). As used herein, “sequencing-by-binding” refers to a sequencing technique wherein specific binding of a polymerase and cognate nucleotide to a primed template nucleic acid molecule (e.g., blocked primed template nucleic acid molecule) is used for identifying the next correct nucleotide to be incorporated into the primer strand of the primed template nucleic acid molecule. The specific binding interaction need not result in chemical incorporation of the nucleotide into the primer. In some embodiments, the specific binding interaction can precede chemical incorporation of the nucleotide into the primer strand or can precede chemical incorporation of an analogous, next correct nucleotide into the primer. Thus, detection of the next correct nucleotide can take place without incorporation of the next correct nucleotide. As used herein, the “next correct nucleotide” (sometimes referred to as the “cognate” nucleotide) is the nucleotide having a base complementary to the base of the next template nucleotide. The next correct nucleotide will hybridize at the 3′-end of a primer to complement the next template nucleotide. The next correct nucleotide can be, but need not necessarily be, capable of being incorporated at the 3′ end of the primer. For example, the next correct nucleotide can be a member of a ternary complex that will complete an incorporation reaction or, alternatively, the next correct nucleotide can be a member of a stabilized ternary complex that does not catalyze an incorporation reaction. A nucleotide having a base that is not complementary to the next template base is referred to as an “incorrect” (or “non-cognate”) nucleotide.

Flow cells provide a convenient format for housing an array of clusters produced by the methods described herein, in particular when subjected to an SBS or other detection technique that involves repeated delivery of reagents in cycles. For example, to initiate a first SBS cycle, one or more labeled nucleotides and a DNA polymerase in a buffer, can be flowed into/through a flow cell that houses an array of clusters. The clusters of an array where primer extension causes a labeled nucleotide to be incorporated can then be detected. Optionally, the nucleotides can further include a reversible termination moiety that temporarily halts further primer extension once a nucleotide has been added to a primer. For example, a nucleotide analog having a reversible terminator moiety can be added to a primer such that subsequent extension cannot occur until a deblocking agent (e.g., a reducing agent) is delivered to remove the moiety. Thus, for embodiments that use reversible termination, a deblocking reagent (e.g., a reducing agent) can be delivered to the flow cell (before, during, or after detection occurs). Washes can be carried out between the various delivery steps as needed. The cycle can then be repeated N times to extend the primer by N nucleotides, thereby detecting a sequence of length N. Example SBS procedures, fluidic systems and detection platforms that can be readily adapted for use with an array produced by the methods of the present disclosure are described, for example, in Bentley et al., Nature 456:53-59 (2008), US 2018/0274024, WO 2017/205336, US 2018/0258472, each of which are incorporated herein in their entirety for all purposes.

In embodiments, sequencing includes a plurality of sequencing cycles. In embodiments, sequencing includes 20 to 100 sequencing cycles. In embodiments, sequencing includes 50 to 100 sequencing cycles. In embodiments, sequencing includes 50 to 300 sequencing cycles. In embodiments, sequencing includes 50 to 150 sequencing cycles. In embodiments, sequencing includes 50 to 100 sequencing cycles. In embodiments, sequencing includes at least 10, 20, 30 40, or 50 sequencing cycles. In embodiments, sequencing includes at least 10 sequencing cycles. In embodiments, sequencing includes 10 to 20 sequencing cycles. In embodiments, sequencing includes 10, 11, 12, 13, 14, or 15 sequencing cycles. In embodiments, sequencing includes (a) extending a sequencing primer by incorporating a labeled nucleotide, or labeled nucleotide analogue and (b) detecting the label to generate a signal for each incorporated nucleotide or nucleotide analogue.

In embodiments, sequencing includes extending a sequencing primer to generate a sequencing read. In embodiments, sequencing includes extending a sequencing primer by incorporating a labeled nucleotide, or labeled nucleotide analogue and detecting the label to generate a signal for each incorporated nucleotide or nucleotide analogue. In embodiments, the labeled nucleotide or labeled nucleotide analogue includes a reversible terminator moiety.

Use of the sequencing method outlined above is a non-limiting example, as essentially any sequencing methodology which relies on successive incorporation of nucleotides into a polynucleotide chain can be used. Suitable alternative techniques include, for example, pyrosequencing methods, FISSEQ (fluorescent in situ sequencing), MPSS (massively parallel signature sequencing), or sequencing by ligation-based methods.

In embodiments, the methods provided herein include sequencing that further includes (a) producing a plurality of sequencing reads; (b) aligning a portion of each sequencing read to a reference sequence; and (c) grouping sequencing reads that belong to the same strand of an original sample polynucleotide based on the aligning and sequences of the barcode sequences.

In embodiments, the methods of making tagged complements provided herein include any sequencing method known to those skilled in the art and include for example, sequencing by synthesis, pyrosequencing, combinatorial probe anchor synthesis, sequencing by ligation, and nanopore sequencing. In embodiments, the sequencing reaction includes sequencing by synthesis, sequencing by ligation, or pyrosequencing. In embodiments, the sequencing reaction includes sequencing by synthesis. In embodiments, the sequencing reaction includes sequencing by ligation. In embodiments, the sequencing reaction includes pyrosequencing. In embodiments, the sequencing reaction includes sequencing by binding.

In embodiments, the methods of making and sequencing tagged complements provided herein include producing a plurality of sequencing reads. In embodiments, each sequencing read includes at least a portion (e.g., a barcode sequence) of two or more interposing oligonucleotide barcodes, or complements thereof. In embodiments, each sequencing read includes at least a portion (e.g., a barcode sequence) of three or more interposing oligonucleotide barcodes, or complements thereof. In embodiments, each sequencing read includes two or more interposing oligonucleotide barcodes, or complements thereof. In embodiments, each sequencing read includes three or more interposing oligonucleotide barcodes, or complements thereof. In embodiments, each sequencing read includes a portion of two or more interposing oligonucleotide barcodes, or complements thereof. In embodiments, each sequencing read includes a portion of two or more interposing oligonucleotide barcodes, or complements thereof. In embodiments, each sequencing read includes at least a portion of three interposing oligonucleotide barcodes, or complements thereof.

In embodiments, the methods of making and sequencing tagged complements provided herein include aligning a portion of each sequencing read to a reference sequence. General methods for performing sequence alignments are known to those skilled in the art. Examples of suitable alignment algorithms, include but are not limited to Burrows-Wheeler Aligner (BWA), Bowtie, the Needleman-Wunsch algorithm (see e.g. the EMBOSS Needle aligner available at www.ebi.ac.uk/Tools/psa/emboss_needle/, optionally with default settings), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.uk/Tools/psa/emboss_water/, optionally with default settings). Optimal alignment may be assessed using any suitable parameters of a chosen algorithm, including default parameters. In embodiments, the reference sequence is a reference genome. In embodiments, the methods of sequencing a template nucleic acid further include generating overlapping sequence reads and assembling them into a contiguous nucleotide sequence of a nucleic acid of interest. Assembly algorithms known in the art can align and merge overlapping sequence reads generated by methods of several embodiments herein to provide a contiguous sequence of a nucleic acid of interest. A person of ordinary skill in the art will understand which sequence assembly algorithms or sequence assemblers are suitable for a particular purpose taking into account the type and complexity of the nucleic acid of interest to be sequenced (e.g. genomic, PCR product, or plasmid), the number and/or length of deletion products or other overlapping regions generated, the type of sequencing methodology performed, the read lengths generated, whether assembly is de novo assembly of a previously unknown sequence or mapping assembly against a backbone sequence, etc. Furthermore, an appropriate data analysis tool will be selected based on the function desired, such as alignment of sequence reads, base-calling and/or polymorphism detection, de novo assembly, assembly from paired or unpaired reads, and genome browsing and annotation. In several embodiments, overlapping sequence reads can be assembled by sequence assemblers, including but not limited to ABySS, AMOS, Arachne WGA, CAP3, PCAP, Celera WGA Assembler/CABOG, CLC Genomics Workbench, CodonCode Aligner, Euler, Euler-sr, Forge, Geneious, MIRA, miraEST, NextGENe, Newbler, Phrap, TIGR Assembler, Sequencher, SeqMan NGen, SHARCGS, SSAKE, Staden gap4 package, VCAKE, Phusion assembler, Quality Value Guided SRA (QSRA), Velvet (algorithm), SPAdes, and the like. It will be understood that overlapping sequence reads can also be assembled into contigs or the full contiguous sequence of the nucleic acid of interest by available means of sequence alignment, computationally or manually, whether by pairwise alignment or multiple sequence alignment of overlapping sequence reads. Algorithms suited for short-read sequence data may be used in a variety of embodiments, including but not limited to Burrows-Wheeler Aligner (BWA), Cross_match, ELAND, Exonerate, MAQ, Mosaik, RMAP, SHRiMP, SOAP, SPAdes, SSAHA2, SXOligoSearch, ALLPATHS, Edena, Euler-SR, SHARCGS, SHRAP, SSAKE, VCAKE, Velvet, PyroBayes, PbShort, and ssahaSNP. In embodiments, aligning to a reference sequence is useful to validate the approaches described herein.

In embodiments, the methods of making and sequencing tagged complements provided herein further include forming a consensus sequence for reads having the same interposing oligonucleotide barcode, or a portion thereof (e.g., a barcode sequence). In embodiments, the consensus sequence is obtained by comparing all sequencing reads aligning at a given nucleotide position (optionally, only among those reads identified as originating from the same sample polynucleotide molecule), and identifying the nucleotide at that position as the one shared by a majority of the aligned reads.

In embodiments, the methods of making and sequencing tagged complements described herein further include computationally reconstructing sequences of a plurality of individual strands of original sample polynucleotides by removing interposing oligonucleotide barcode-derived sequences and joining sequences for adjacent portions of the sample polynucleotide. Reconstruction can be performed on individual reads, or on consensus sequences produced from those reads. In embodiments, the methods of making and sequencing tagged complements described herein further include aligning computationally reconstructed sequences.

A variety of suitable sequencing platforms are available for implementing methods disclosed herein (e.g., for performing the sequencing reaction). Non-limiting examples include SMRT (single-molecule real-time sequencing), ion semiconductor, pyrosequencing, sequencing by synthesis, combinatorial probe anchor synthesis, SOLiD sequencing (sequencing by ligation), and nanopore sequencing. Sequencing platforms include those provided by Illumina® (e.g., the HiSeq™, MiSeq™ and/or Genome Analyzer™ sequencing systems); Ion Torrent™ (e.g., the Ion PGM™ and/or Ion Proton™. sequencing systems); Pacific Biosciences (e.g., the PACBIO RS II sequencing system); Life Technologies™ (e.g., a SOLiD sequencing system); Roche (e.g., the 454 GS FLX+ and/or GS Junior sequencing systems). See, for example U.S. Pat. Nos. 7,211,390; 7,244,559; 7,264,929; 6,255,475; 6,013,445; 8,882,980; 6,664,079; and 9,416,409.

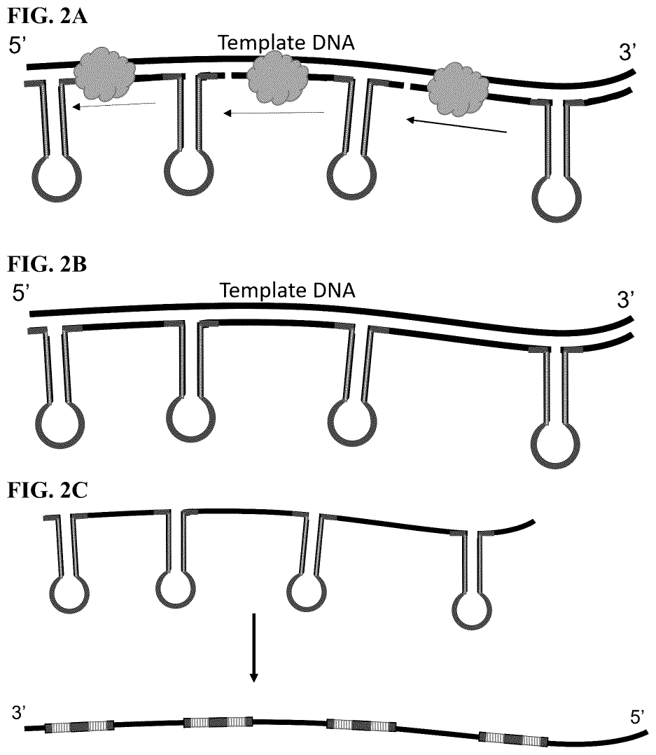

In an aspect is provided a method of sequencing a target nucleic acid. In embodiments, the method includes combining a sample polynucleotide (e.g., a polynucleotide containing the target nucleic acid sequence), hybridizing a plurality of interposing oligonucleotide barcodes (e.g., the interposing oligonucleotide barcodes as described herein) to the sample polynucleotide, extending the 3′ ends of the hybridization sequence (e.g., the available second hybridization sequence) with a polymerase to create an extension product, ligating the 3′ end of the extension product with the 5′ end of an adjacent hybridization sequence (e.g., the first hybridization sequence of an adjacent interposing oligonucleotide barcode) hybridized to the sample polynucleotide to generate a complement of the sample polynucleotide including a plurality of interposing oligonucleotide barcodes (see for example FIG. 2C), amplifying the complement to generate an amplified product, fragmenting the amplified product to produce fragments, sequencing the fragments to produce a plurality of sequence reads, assembling the sequence reads to produce an assembled sequence of the target nucleic acid. In embodiments, following fragmentation, the fragments are subjected to standard library preparation methods as known to those skilled in the art and described herein. For example, the method includes ligating adapters (e.g., platform specific oligonucleotide sequences) to the fragments, amplifying the resultant products (i.e., the fragments containing adapters) to generate a plurality of polynucleotides.

In embodiments, assembling the sequence reads includes grouping the sequencing reads based on co-occurrence of barcode sequences of the interposing oligonucleotide barcodes. In embodiments, the assembling further includes aligning the reads within each group that belong to the same strand of an original sample polynucleotide based on the sequences of the barcode sequences.

### EXAMPLES

### Example 1: Single-Cell Paired Heavy- and Light-Chain Antibody Sequencing

Described herein are methods pertaining to sequencing two independent, distinct nucleic acids. Traditional sequencing-by-synthesis (SBS) methodologies employ serial incorporation and detection of labeled nucleotide analogues. For example, high-throughput SBS technology (see, for example, Bentley D R, et al. Nature, 2008, 456, 53-59) uses cleavable fluorescent nucleotide reversible terminator (NRT) sequencing chemistry (see, for example, see U.S. Pat. No. 6,664,079; or Ju et al. Proc. Natl. Acad. Sci. USA, 2006, 103, 19635-19640). These cleavable fluorescent NRTs were designed based on the following rationale: each of the four nucleotide types (dA, dC, dG, dT, and/or dU) is modified by attaching a unique cleavable fluorophore to the specific location of the nucleobase and capping the 3′-OH group of the nucleotide sugar with a small reversible moiety (also referred to herein as a reversible terminator) so that they are still recognized by DNA polymerase as substrates. The reversible terminator temporarily halts the polymerase reaction after nucleotide incorporation while the fluorophore signal is detected. After incorporation and signal detection, the fluorophore and the reversible terminator is cleaved to resume the polymerase reaction in the next cycle. These traditional SBS techniques require de novo assembly of relatively short lengths of DNA (e.g., 35 to 300 base pairs), which makes resolving complex regions with mutations or repetitive sequences difficult. The application of those technologies to de-novo genome assemblies is limited by short sequence read length, which, by previous methods, is insufficient to resolve complex genome structure and to produce consistent genome assembly. To address these limitations, researchers typically supplement short read sequencing data (e.g., short read sequencing data having an error rate of less than about 1.5%) with data from long read sequencers (e.g., read length 10 kb, error rate 10-15%). Further, it is difficult to reliable obtain phasing data (i.e., which variants are on the same chromosome) or detecting structural variants from short read data. Described herein are methods for achieving greater read lengths by utilizing specialized interposing oligonucleotide barcodes.

Inheritance patterns of genetic variation in complex traits may be influenced by interactions among multiple genes and alleles across long distances. Examination of the gene pairs encoding the two chains including adaptive immune receptors from individual cells to accurately determine the complete repertoires of immune receptors expressed in patients are critical for a greater understanding of many biotechnology and medical applications. Experiments herein demonstrate that long-ranged nucleic acid sequencing of linked transcripts can be performed.

The functions of immune cells such as B- and T-cells are predicated on the recognition through specialized receptors of specific targets (antigens) in pathogens. There are approximately 10¹⁰ to 10¹¹ B-cells and approximately 10¹¹ T-cells in an adult human (see, for example, Ganusov V V, De Boer R J. Trends Immunol. 2007; 28(12):514-8; and Bains I, Antia R, Callard R, Yates A J. Blood. 2009; 113(22):5480-5487). Immune cells are critical components of adaptive immunity and directly bind to pathogens through antigen-binding regions present on the cells. Within lymphoid organs (e.g., bone marrow for B cells and the thymus for T cells) the gene segments variable (V), joining (J), and diversity (D) rearrange to produce a novel amino acid sequence in the antigen-binding regions that allow for the recognition of antigens from a range of pathogens (e.g., bacteria, viruses, parasites, and worms) as well as antigens arising from cancer cells. The large number of possible V-D-J segments, combined with additional (junctional) diversity, lead to a theoretical diversity of >10¹⁴, which is further increased during adaptive immune responses. Overall, the result is that each B- and T-cell expresses a highly variable receptor, whose sequence is the outcome of both germline diversity and somatic recombination. Somatic recombination is a process that creates new combinations of V, D and J segments via a complicated mechanism that involves gene excision and alternative splicing. These antibodies also contain a constant (C) region, which confers the isotype to the antibody. In most mammals, there are five antibody isotypes: IgA, IgD, IgE, IgG, and IgM. For example, each antibody in the IgA isotype shares the same constant region. Characterization of an individual's immune repertoire (i.e., the global profile of which immune cell receptors are present in an individual), requires full length sequencing of the recombined VDJ region, which is difficult to determine with short read sequencing data. Thus, obtaining long-range sequence data is incredibly insightful to gain insights into the adaptive immune response in healthy individuals and in those with a wide range of diseases.

A mature antibody consists of two identical heavy chains (HC) linked through disulfide bonds and two identical light chains (LC) each linked to one of the HC, generating two identical antigen-binding sites formed by the first immunoglobulin (Ig) domain of each chain pair (Schroeder H W, Cavacini L. J. Allergy Clin. Immunol. 2010; 125:S41-S52). The HC and LC are encoded in separate gene loci, and each B cell normally expresses a single functional HC and LC sequence. Existing antibody repertoire analysis has primarily focused on bulk analysis of HC sequences, lacking the native LC pairing information that is necessary for antibody cloning and expression (Georgiou G et al. Nat. Biotechnol. 2014; 32:158-68).

The development of a high-throughput method for interrogating paired HC and LC sequences facilitates antibody discovery, vaccine efficacy studies, and other healthcare applications. Emerging techniques for paired HC and LC variable domain sequencing vary in complexity and efficiency, see for example CelliGO (Gerard A et al. Nat. Biotechnol. 2020; 38:715-721), and 10× Genomics' Chromium protocol (Goldstein L D et al. Comms. Bio. 2019; 2:304), yet are limited in the amount of information they can provide. Described herein are methods for determining long-range, paired Ig HC and LC sequences at single-cell resolution using NGS technologies, capable of sequencing millions of B-cells or T-cells in a single experiment (e.g., on a single array).

Interposing barcode: The methods described herein feature a plurality of interposing barcodes to sequence the entirety of the paired HC and LC sequence. Briefly, an example interposing barcode is shown in FIG. 1A, and includes a loop region, a stem region, and two hybridization sequences. The loop region includes about 10 to about 20 random nucleotides (e.g., AGCCTGCCTG (SEQ ID NO:8)). Such random sequences may be referred to as molecular barcodes or unique molecular identifiers (UMI). In embodiments of the methods described herein, synthetic long reads are constructed by grouping together UMIs based on direct or indirect co-occurrence in the library, and then assembling the reads back into the original full-length molecule. In embodiments, the length of the UMI is optimized based on the total number of insertions sites (number of targeted molecules X number of insertion locations) to reduce the incorporation of two of the same UMIs in different molecules, while maximizing the amount of sequence in the read that is from the target molecule. Rare instances where the same UMI is observed in two different molecules can be addressed bioinformatically.

Aside from forming the backbone for long read alignment, the introduction of UMIs into sequencing libraries prior to target amplification by PCR has been shown to dramatically increase the sensitivity for rare mutations and enable absolute read counting. The stem region includes two known sequences capable of hybridizing to each other, ranging from about 5 to about 10 nucleotides, and is stable (i.e., capable to remaining hybridized together) at approximately a maximum temperature of 37° C., and unhybridizes (i.e., denatures) at temperatures greater than 50° C. Finally, the hybridization sequences are each about 9 to about 15 nucleotides (e.g., AGTCG for pad 1, and GGGAG for pad 2) and are capable of hybridizing to single stranded template nucleic acids (i.e., they are a complement to the original target). The sequences of the hybridization sequence may be random or may include a targeted priming sequence to maximize placement of the IBC. FIG. 1B depicts the interposing barcode when the stem regions are denatured. In embodiments, only Type 1 interposing barcodes are used. In other embodiments, only Type 2 interposing barcodes are used. Alternatively, the hybridization sequences can include targeted priming sequences (e.g., nucleotide sequences that are complementary to regions in the constant region that are interspersed between the V, D, and J regions). In this alternative embodiment, the interposing barcodes (IBCs) have targeted priming sequences in the hybridization sequences, wherein the priming sequences target the constant regions that flank the variable regions.

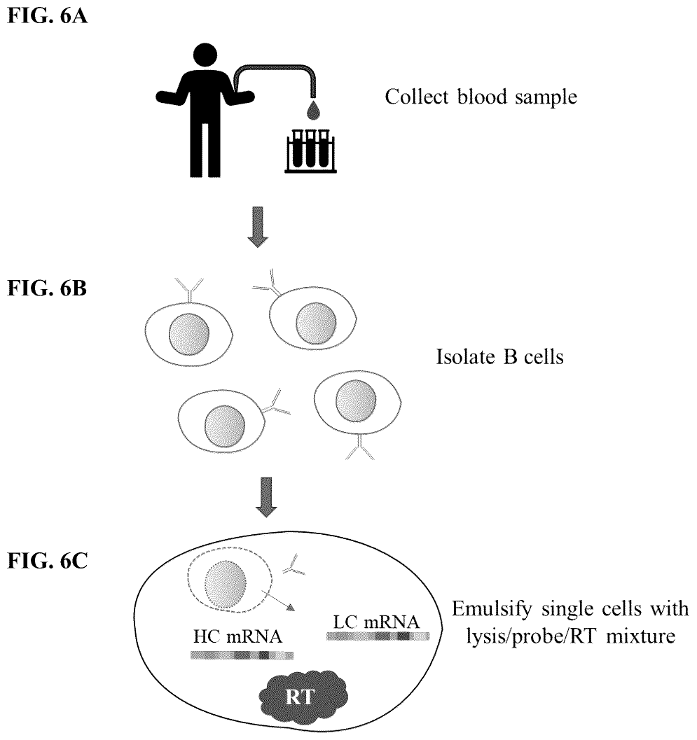

Single-cell isolation and emulsification: In embodiments, methods described herein utilize cells (e.g., PBMCs such as human peripheral blood B-cells, T-cells, and T follicular helper (TFH) cells) isolated from a subject using methods known in art, such as a blood draw (FIG. 6A). In embodiments, the cells may also be extracted and/or isolated from tissue. In some embodiments, cells may optionally be sorted in vitro based on the expressed BCR or other cell marker(s) (FIG. 6B). In embodiments, cell isolation from a subject occurs at temporally-distinct intervals based upon conditions that may modulate the subject's BCR or TCR repertoire, for example, stimulation of the adaptive immune system with a foreign antigen through vaccination. The cells are then emulsified using methods known in the art such that there is one immune cell per droplet. The emulsification mixture contains lysis buffer and amplification components (e.g., RT-PCR reaction components) to enable overlap extension for generating paired HC-LC amplicons (FIG. 6C).

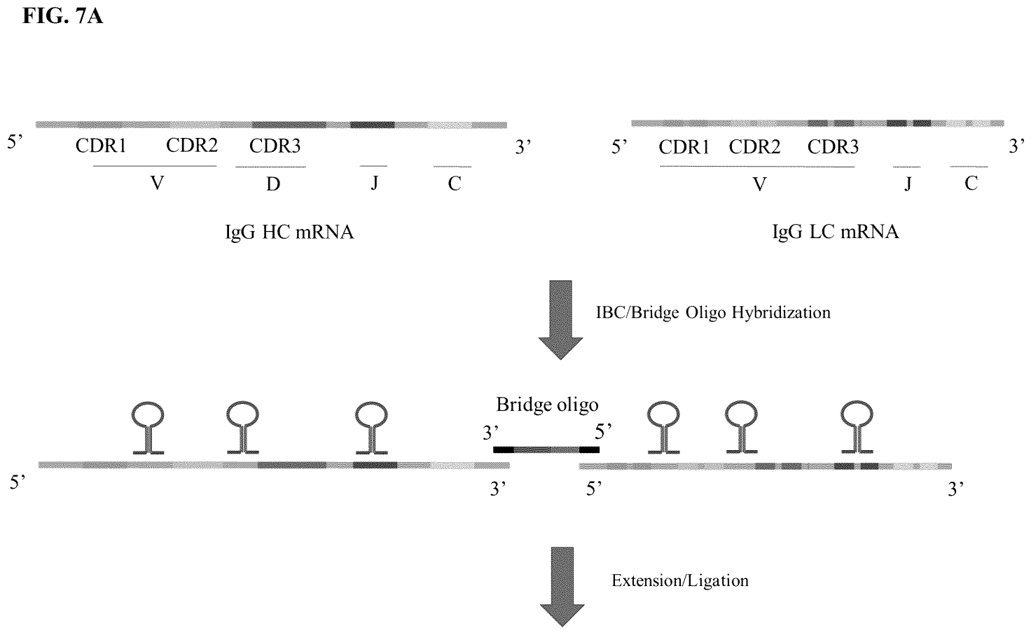

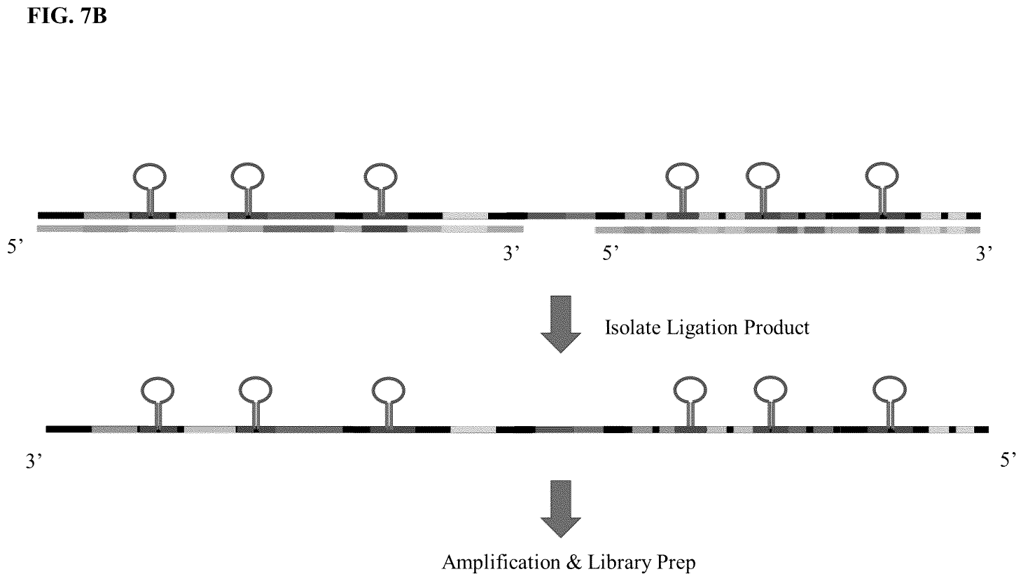

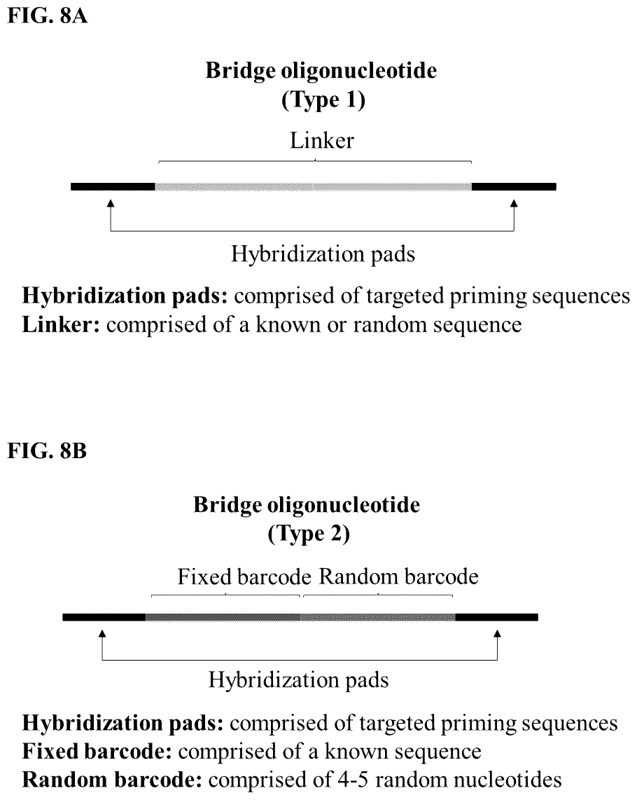

RT-PCR—bridge oligo and IBCs (option 1): Following emulsification and lysis, Ig mRNA, for example, IgG HC and LC mRNA is released within the droplet. The RT-PCR reaction mixture contains the IBCs described intra, and a bridge oligo that hybridizes to both the HC and LC mRNA (FIG. 7A). The bridge oligo may include two hybridization sequences consisting of targeted priming regions flanking a linking region (type 1) (see FIG. 8A). Alternatively, the bridge oligo may include two hybridization sequences consisting of targeted priming regions flanking a fixed barcode region that is 5′ to a random barcode region (type 2) (see FIG. 8B). In embodiments, the random barcode region is 5′ to the fixed barcode region. IBCs are included in the reaction mixture at an appropriate concentration such that there are approximately 50-200 bases between each IBC. Following IBC and bridge oligo hybridization, RT-PCR is performed, followed by ligation as described herein to generate a contiguous molecule (FIG. 7B). A non strand-displacing polymerase (e.g., Klentaq, T4, T7, Bst, Phusion, Tfl, Pfu, or Stoffel fragment) extends the complement strand to generate an extension segment, as shown in FIG. 2A, and a ligase (for example, Taq DNA Ligase, HiFi Taq DNA Ligase, T4 DNA ligase, or Ampligase® DNA Ligase) ligates the ends of the extension segment together with the next interposing barcode to produce a single integrated strand, as depicted in FIG. 2B. For example, a T4 extension-ligation reaction may be carried out by combining the polynucleotide ends, ligation buffer, ATP, T4 DNA ligase, water, and incubating the mixture at between about 20° C. to about 45° C., for between about 5 minutes to about 30 minutes. In embodiments, a T4 extension-ligation reaction may be carried out by combining the polynucleotide ends, ligation buffer, ATP, T4 DNA ligase, water, and incubating the mixture at between about 37° C., for between about 30 minutes to about 90 minutes. In some embodiments, the T4 extension-ligation reaction is incubated at 37° C. for 30 minutes. In some embodiments, the T4 extension-ligation reaction is incubated at 37° C. for 30 to 90 minutes. In some embodiments, the T4 extension-ligation reaction is incubated at 37° C. for 60 minutes. In some embodiments, the T4 extension-ligation reaction is incubated at 45° C. for 30 minutes. In some embodiments, the T4 extension-ligation reaction is incubated at 45° C. for 60 minutes. In embodiments, the ligase reaction is stopped by adding Tris buffer with high EDTA and incubating for 1 minute. The non strand-displacing polymerase can either be a naturally occurring enzyme, or one that is specifically engineered to minimize strand displacement. As described in Example 3, strategies to reduce strand-displacement of the hybridized IBCs during reverse transcription are advantageous for cDNA extension, and can include modified oligonucleotides and/or strand-displacement reverse transcriptase mutants. Following amplicon production, droplets are broken and full-length paired HC-LC cDNA (i.e., the integrated strand) is isolated (FIG. 7B) and prepared for sequencing. For example, the isolated ligation product (i.e., the integrated strand) is fragmented. Following fragmentation, the fragments are end repaired or end polished. Additional sequences such as adapters or primers may then be added using conventional means to permit platform specific sequences or to provide a binding site for sequencing primers. Following adapter ligation, the nucleic acid templates may be purified, amplified, or sequenced using methods known to those skilled in the art.

RT-PCR—constant/variable region primers (option 2): Following emulsification and lysis, Ig mRNA, for example, IgG HC and LC mRNA (FIG. 9A) are released within the droplet. The RT-PCR reaction mixture contains constant region primers for reverse transcription and variable region-specific overlap primers for overlap extension PCR (FIG. 9B). Methods for performing overlap extension RT-PCR for generating paired HC-LC amplicons are known in the art (see, for example, Devulapally P R et al. Genome Medicine 2018; 10:34 and U.S. Pat. Pubs. 2015/0141261 and 2013/0296535, each of which is incorporated herein by reference for all purposes). How sequences are oriented with respect to one another can be controlled by which of the primers in the RT-PCR reaction include the tails of the “linker primers” illustrated in FIG. 9A. Using hybridized overlap oligonucleotides (e.g., a first overlap oligonucleotide and a second overlap oligonucleotide, wherein a sequence at the 5′ end of the first overlap oligonucleotide is hybridized to a sequence at the 5′ end of the second overlap oligonucleotide) and IBCs. IgG HC and LC mRNA are annealed to two hybridized overlap oligonucleotides followed by reverse transcription (e.g., reverse transcription in overlap extension RT-PCR), wherein each overlap oligonucleotide is specific for the variable region of the IgG HC or IgG LC mRNA (FIG. 9A). Following reverse transcription, second strand cDNA synthesis is performed (e.g., RNAse H nicking followed by DNA Polymerase I extension and ligation of the products to form a contiguous cDNA strand). The reverse transcription and second strand cDNA synthesis steps produce a double-stranded bridged polynucleotide (e.g., a double-stranded cDNA bridged polynucleotide) having cDNA sequences of the two mRNA molecules, covalently linked by sequences of the two overlap oligonucleotides. PCR enrichment of the cDNA product may optionally be performed using forward and reverse primers (FIG. 9B). [0001] Accordingly, linking a sequence of a first independent polynucleotide to the sequence of a second independent polynucleotide or a complement thereof can include: (a) linking a sequence from a 5′ end of the first independent polynucleotide to a complement of a sequence from a 5′ end of the second independent polynucleotide, (b) linking a sequence from a 3′ end of the first independent polynucleotide to a complement of a sequence from a 3′ end of the second independent polynucleotide, or (c) linking a sequence from a 3′ end of the first independent polynucleotide to a sequence from a 5′ end of the second independent polynucleotide. Additional information related to performing overlap-extension RT-PCR for linking transcripts may be found in U.S. Pat. No. 7,749,697, which is hereby incorporated by reference for all purposes. Following RT-PCR, droplets are broken and full-length paired HC-LC cDNA is isolated. To this isolated cDNA sample, IBCs (as described herein) are added at an appropriate concentration such that there are approximately 50-200 bases between each IBC (FIG. 9C). A non strand-displacing polymerase (e.g., Klentaq, T4, T7, Bst, Phusion, Tfl, Pfu, or Stoffel fragment) extends the complement strand to generate an extension segment, as shown in FIG. 2A, and a ligase (for example, Taq DNA Ligase, HiFi Taq DNA Ligase, T4 DNA ligase, or Ampligase® DNA Ligase) ligates the ends of the extension segment together with the next interposing barcode to produce a single integrated strand, as depicted in FIG. 9C, and more generally in FIG. 2B. For example, a T4 extension-ligation reaction may be carried out by combining the polynucleotide ends, ligation buffer, ATP, T4 DNA ligase, water, and incubating the mixture at between about 20° C. to about 45° C., for between about 5 minutes to about 30 minutes. In embodiments, a T4 extension-ligation reaction may be carried out by combining the polynucleotide ends, ligation buffer, ATP, T4 DNA ligase, water, and incubating the mixture at between about 37° C., for between about 30 minutes to about 90 minutes. In some embodiments, the T4 extension-ligation reaction is incubated at 37° C. for 30 minutes. In some embodiments, the T4 extension-ligation reaction is incubated at 37° C. for 30 to 90 minutes. In some embodiments, the T4 extension-ligation reaction is incubated at 37° C. for 60 minutes. In some embodiments, the T4 extension-ligation reaction is incubated at 45° C. for 30 minutes. In some embodiments, the T4 extension-ligation reaction is incubated at 45° C. for 60 minutes. In embodiments, the ligase reaction is stopped by adding Tris buffer with high EDTA and incubating for 1 minute. The non strand-displacing polymerase can either be a naturally occurring enzyme, or one that is specifically engineered to minimize strand displacement.

As even “non-strand displacing” DNA polymerases can have a slight ability to displace a DNA oligonucleotide from a template strand of DNA, the hybridization of the oligonucleotide can be enhanced in order to stop strand displacement by the polymerase. Prevention of displacement can be achieved by using modifications to the oligonucleotide itself or by using additives that either stabilize the hybridization of the oligonucleotide or that stop the polymerase. Modifications to the oligonucleotides that reduce or inhibit the strand displacement activity of the polymerase are for instance 2′ fluoro nucleosides, PNAs (peptide nucleic acids), ZNAs (zip nucleic acids), G-Clamps (U.S. Pat. No. 6,335,439, a cytosine analogue capable of Clamp Binding to Guanine) or LNAs (US 2003/0092905; U.S. Pat. No. 7,084,125). In embodiments, the non strand-displacing polymerase activity can be inhibited by the addition of Actinomycin D. Actinomycin D can be added to the reaction in sufficient amounts to avoid to reduce strand displacement of the polymerase as compared without actinomycin addition. In embodiments, Actinomycin D is added at about 50 μg/ml.

Optionally, the template DNA sample is washed away, and the resultant integrated strand may be subjected to reaction conditions (e.g., elevated temperature or denaturing additives) such that the stem regions of interposing barcodes and/or any secondary structures present denature to form a linear integrated strand, as schematically shown in FIG. 2C. The integrated strand may be amplified using methods known to those skilled in the art (e.g., standard PCR amplification or rolling circle amplification) and subjected to standard library preparation methods as known to those skilled in the art and described herein. Alternatively, the cDNA synthesis occurs in the presence of dUTP such that the template is enzymatically degraded. For example, cleavage and degradation at dUTP sites may be achieved using uracil DNA glycosylase and endonuclease VIII (USER™, NEB, Ipswich, Mass.), as described in U.S. Pat. No. 7,435,572. The integrated strand may serve as the input DNA with any commercially available library preparation kit. A variety of kits for making sequencing libraries from DNA are available commercially. The original template strand does not necessarily need to be removed and washed away. For example, in some applications it may be useful and convenient to take the template strands all the way through the sequencing steps and provide useful information in addition to the IBC tagged strand. Library preparation methods are briefly summarized herein (e.g., see Example 3 for additional details).

The integrated strand may be fragmented using techniques known to those in the art. Three approaches available to fragment nucleic acid chains include: physical, enzymatic, and chemical. DNA fragmentation is typically done by physical methods (i.e., acoustic shearing and sonication) or enzymatic methods (i.e., non-specific endonuclease cocktails and transposase tagmentation reactions). Following fragmentation, the DNA fragments are end repaired or end polished. Typical polishing mixtures contain T4 DNA polymerase and T4 polynucleotide kinase. These enzymes excise 3′ overhangs, fill in 3′ recessed ends, and remove any potentially damaged nucleotides thereby generating blunt ends on the nucleic acid fragments. The T4 polynucleotide kinase used in the polishing mix adds a phosphate to the 5′ ends of DNA fragments that can be lacking such, thus making them ligation-compatible to NGS adapters. Generally, a single adenine base is added to form an overhang via an A-tailing reaction. This “A” overhang allows adapters containing a single thymine overhanging base to base pair with the DNA fragments. Additional sequences such as adapters or primers may then be added using conventional means to permit platform specific sequences or to provide a binding site for sequencing primers. Following adapter ligation, the nucleic acid templates may be purified, amplified, or sequenced using methods known to those skilled in the art.

For example, the following protocol is then followed to prepare the integrated strand for sequencing on next generation sequencing devices. The input DNA (i.e., the integrated strand) is fragmented to make small DNA molecules with a modal size of about 100 to about 400 base pairs with random ends. This is done by sonication, chemical fragmentation, or enzymatic fragmentation. The resulting DNA fragments generated by sonication are end polished to produce a library of DNA fragments with blunt, 5′-phosphorylated ends that are ready for ligation. The end polishing is accomplished by using the T4 DNA polymerase, which can fill in 5′ overhangs via its polymerase activity and recess 3′ overhangs via its 3′→5′ exonuclease activity. The phosphorylation of 5′ ends is accomplished by T4 polynucleotide kinase.

Adapter ligation: Ligation of double-stranded DNA adapters is accomplished by use of T4 DNA ligase. Depending on the adapter, some double-stranded adapters may not have 5′ phosphates and contain a 5′ overhang on one end to prevent ligation in the incorrect orientation.

Now the adapter-ligated library may be size-selected (e.g., selecting for approximately 200-250 basepair size range). By doing this, unligated adapters and adapter dimers are removed, and the optimal size-range for subsequent PCR and sequencing is selected. Adapter dimers are the result of self-ligation of the adapters without an insert sequence. These dimers form clusters very efficiently and consume valuable space on the flow cell without generating any useful data. Thus, known cleanup methods may be used, such as magnetic bead-based clean up, or purification on agarose gels.

The resultant strand is then subjected to a nucleic acid sequencing reaction using any available sequencing technology. Current SBS platforms use clonal amplification of the initial template molecules with a cluster (i.e., PCR colonies, referred to as polonies) to increase the signal-to-noise ratio because the systems are not sensitive enough to detect the extension of one base at the individual DNA template molecule level. Standard amplification methods employed in commercial sequencing devices (e.g., solid-phase bridge amplification) typically amplify a template using surface immobilized primers to produce a plurality of double-stranded nucleic acid molecules, wherein at least one strand of each double-stranded nucleic acid molecule is attached to the solid support at its 5′ ends. A common method of doing solid-phase amplification involves bridge amplification methodologies (referred to as bridge PCR) as exemplified by the disclosures of U.S. Pat. Nos. 5,641,658; 7,115,400; 7,790,418; U.S. Patent Publ. No. 2008/0009420, each of which is incorporated herein by reference in its entirety. In sum, bridge amplification methods allow amplification products (e.g., amplicons) to be immobilized on a solid support in order to form arrays comprised of colonies (or “clusters”) of immobilized nucleic acid molecules. Each cluster or colony on such an array is formed from a plurality of identical immobilized polynucleotide strands and a plurality of identical immobilized complementary polynucleotide strands. The products of solid-phase amplification reactions are referred to as “bridged” structures when formed by annealed pairs of immobilized polynucleotide strands and immobilized complementary strands, both strands being immobilized on the solid support at the 5′ end, preferably via a covalent attachment. During bridge PCR, additional chemical additives may be included in the reaction mixture, in which the DNA strands are denatured by flowing a denaturant such as formamide or NaOH with the DNA, which chemically denatures complementary strands. This is followed by washing out the denaturant and reintroducing a polymerase in buffer conditions that allow primer annealing and extension.

The resultant strand is then subjected to library preparation and nucleic acid sequencing reactions using any available sequencing technology. A variety of sequencing methodologies can be used such as sequencing-by synthesis (SBS), pyrosequencing, sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568; and. 6,274,320, each of which is incorporated herein by reference in its entirety). In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. A plurality of different nucleic acid fragments that have been attached at different locations of an array can be subjected to an SBS technique under conditions where events occurring for different templates can be distinguished due to their location in the array. In embodiments, the sequencing step includes annealing and extending a sequencing primer to incorporate a detectable label that indicates the identity of a nucleotide in the target polynucleotide, detecting the detectable label, and repeating the extending and detecting steps. In embodiments, the methods include sequencing one or more bases of a target nucleic acid by extending a sequencing primer hybridized to a target nucleic acid (e.g., an amplification product produced by the amplification methods described herein). In embodiments, the sequencing step may be accomplished by a sequencing-by-synthesis (SBS) process. In embodiments, sequencing includes a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 10,738,072, 7,541,444 and 7,057,026. Once such a modified nucleotide has been incorporated into the growing polynucleotide chain complementary to the region of the template being sequenced, there is no free 3′-OH group available to direct further sequence extension and therefore the polymerase cannot add further nucleotides. Once the identity of the base incorporated into the growing chain has been determined, the 3′ block may be removed to allow addition of the next successive nucleotide. By ordering the products derived using these modified nucleotides it is possible to deduce the DNA sequence of the DNA template.

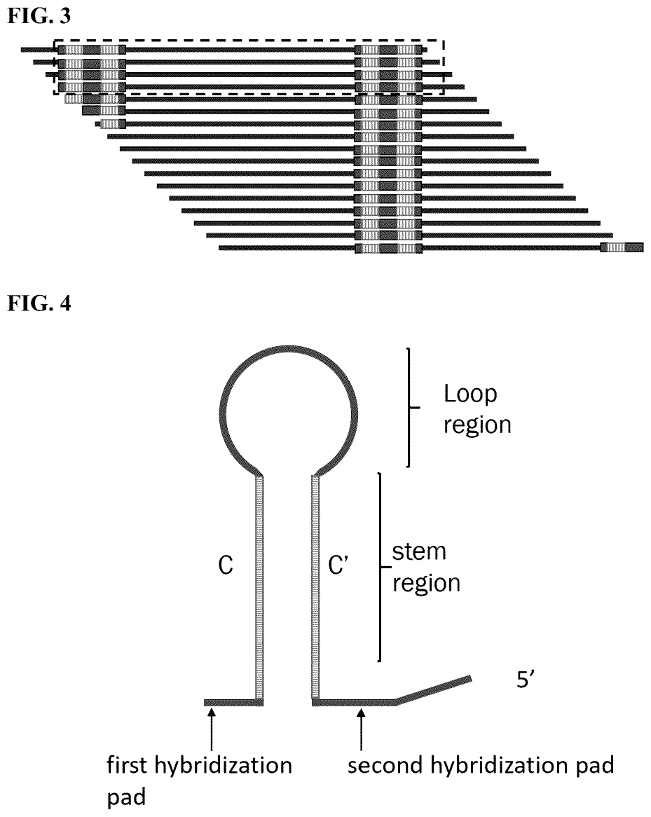

In embodiments, single-end (i.e., sequencing of the sense or anti-sense strand) or paired-end sequencing (i.e., sequencing of the sense and anti-sense strand) is performed. In embodiments of paired end sequencing, the first sequencing read being about 50 bases or less, and the second sequencing read being about 250 bases or less. In embodiments of paired end sequencing, the first sequencing read being about 100 bases or less, and the second sequencing read being about 200 bases or less. In embodiments of paired end sequencing, the first sequencing read being about 150 bases or less, and the second sequencing read being about 150 bases or less. In some embodiments, the first sequencing read is about 35 bases or less. In embodiments, the second sequencing read is about 500 bases or less. In embodiments, the second sequencing read is about 1000 bases or less. Once data is available from the sequencing reaction, initial processing (often termed “pre-processing”) of the sequences is typically employed prior to annotation. Pre-processing includes filtering out low quality sequences, sequence trimming to remove continuous low quality nucleotides, merging paired-end sequences, or identifying and filtering out PCR repeats using known techniques in the art. The sequenced reads may then be assembled and aligned using bioinformatic algorithms known in the art (e.g., in addition to the workflows illustrated in FIG. 3 and depicted in FIG. 10).

In embodiments, the methods described supra may be adapted for paired T-cell receptor (TCR) alpha and beta chain single-cell sequencing. The genes encoding alpha (TCRA) and beta (TCRB) chains are composed of multiple non-contiguous gene segments which include V, D, and J segments for TCRB and V and J for TCRA. As with B cell receptor diversity, the enormous diversity of TCR repertoires is generated by random combinatorial gene events. The methods described here can be used to provide a detailed, single cell view of TCR diversity in T-cells.

### Example 2: SARS-CoV-2 Neutralizing Antibody Discovery

The immune system generates a vast repertoire of antibodies in response to infection or immunization that can potentially be explored for diagnostic, therapeutic, or research applications. The phenotypic diversity of target-specific IgGs in an individual underlies protection following vaccination or infection (Gerard A et al. Nat. Biotechnol. 2020; 38:715-721). Antibodies can block viral infection at any number of steps in the process of virus entry. Antibody-mediated neutralization of viruses is the direct inhibition of viral infectivity resulting from antibody docking to virus particles (VanBlargan L A et al. Microbiol. Mol. Biol. Rev. 2016; 80(4): 989-1010). The elicitation of a neutralizing-antibody (NAb) response is a correlate of protection for many vaccines and contributes to long-lived protection against many viral infections. Enriching for epitope-specific NAbs and characterizing their functionality directly informs vaccine design and potential therapeutic development.

As discussed in Example 1, obtaining paired HC-LC sequencing information is useful for subsequent antibody cloning and expression. Using the methods described herein, the IgG HC-LC repertoire of a subject can be interrogated, for example, pre- and post-immunization with a vaccine or novel antigen. The severe acute respiratory syndrome coronavirus-2 (SARS-CoV-2) spike protein receptor binding domain (RBD) binds to the ACE2 cell receptor to initiate membrane fusion and entry (Lan J et al. Nature 2020; 581:215-220). Using methods known in the art, a subject is immunized with a viral antigen, for example, purified fragments of the SARS-CoV-2 spike protein, or a non-antigenic control.

Following vaccination, B-cells are isolated, emulsified into single-cell droplets, and subjected to paired HC-LC sequencing as described herein and in Example 1. Bioinformatic analysis of the resulting sequencing data reveals specific HC-LC pairs that were upregulated in B-cells following immunization with the viral antigen (e.g., the SARS-CoV-2 spike protein fragment). Those HC-LC pairs showing the highest expression can then be cloned and expressed in either mammalian or bacterial cells and purified using standard molecular cloning and antibody purification techniques. The panel of NAbs can then be subjected to additional analyses such as a binding assay to either SARS-CoV-2 spike protein, or intact viral particles, to determine antibody binding affinity. Additional viral neutralization assays can be performed, as is known in the art. The methods described herein help to rapidly isolate and develop potent NAbs against emerging pathogens to guide vaccine and therapeutic development.

### Example 3: Library Preparation and Nucleic Acid Workflow

DNA Library Preparation is performed according to known methods in the art, e.g., described elsewhere and briefly below. For whole genome workflows, genomic DNA is tethered to an affinity tag (e.g., biotinylated) using known techniques in the art. For example, biotin containing dideoxynucleotide triphosphates (biotin-ddNTP) are added in the presence of a non strand-displacing DNA polymerase (e.g., Klentaq, T4, T7, Bst, Phusion, Tfl, Pfu, or Stoffel fragment) or terminal transferase (TdT) such that the input genomic DNA is biotinylated on the 3′ ends. Next, the double stranded biotinylated DNA is subjected to denaturing conditions (e.g., elevated temperature or NaOH, followed by neutralization) and attached to a complementary affinity (e.g., streptavidin) decorated bead. The biotin reacts to covalently attach the 3′ end of the single strand DNA.

Sample interposing barcodes (as described herein) are added at an appropriate concentration such that there are approximately 50-200 bases between each hybridized IBC. A non strand-displacing polymerase (e.g., Klentaq, T4, T7, Bst, Phusion, Tfl, Pfu, or Stoffel fragment) extends the complement strand to generate an extension segment, as shown in FIG. 2A, and a ligase (e.g., T4 DNA ligase, Ampligase, Tth ligase, T7 ligase, E. coli DNA ligase, 9° N™ DNA Ligase (NEB), or Taq Ligase) ligates the ends of the extension segment together with the next interposing barcode to produce a single integrated strand, as depicted in FIG. 2B. As non strand-displacing DNA polymerases have a slight ability to displace a DNA oligonucleotide from a template strand, the hybridization of the oligonucleotide can be enhanced in order to stop strand displacement by the polymerase.

Alternatively, the loop region of an IBC includes a modified nucleotide that contains an affinity tag (e.g., a biotin containing nucleotide). A mixture of modified IBCs and non-modified IBCs are added are added at an appropriate concentration such that there are approximately 50-200 bases between each hybridized IBC. A non strand-displacing polymerase (e.g., Klentaq, T4, T7, Bst, Phusion, Tfl, Pfu, or Stoffel fragment) extends the complement strand to generate an extension segment, as shown in FIG. 2A, and a ligase (e.g., T4 DNA ligase, Ampligase, Tth ligase, T7 ligase, E. coli DNA ligase, 9° N™ DNA Ligase (NEB), or Taq Ligase) ligates the ends of the extension segment together with the next interposing barcode to produce a single integrated strand, as depicted in FIG. 2B. As non strand-displacing DNA polymerases have a slight ability to displace a DNA oligonucleotide from a template strand, the hybridization of the oligonucleotide can be enhanced in order to stop strand displacement by the polymerase. The modified IBC reacts with a complementary affinity tag (e.g., streptavidin) decorated bead to immobilize the nucleic acid sequence.

The template DNA sample may be washed away, and the resultant integrated strand (i.e., the complementary strand containing a plurality of adapters) may be subjected to reaction conditions (e.g., elevated temperature or denaturing additives) such that the stem regions of interposing barcodes and/or any secondary structures present denature to form a linear integrated strand, as schematically shown in FIG. 2C. The integrated strand is then converted to double stranded DNA (e.g., (e.g., Single Strand Adapter Library Prep (SALP) or by ss-DNA ligation using a circligase) and amplified using known techniques in the art.

An alternative workflow is presented wherein the original template is not washed away. In this workflow, genomic DNA is denatured and IBCs are added at an appropriate concentration such that there are approximately 50-200 bases between each hybridized IBC. A non strand-displacing polymerase (e.g., Klentaq, T4, T7, Bst, Phusion, Tfl, Pfu, or Stoffel fragment) extends the complement strand to generate an extension segment, as shown in FIG. 2A, and a ligase (e.g., T4 DNA ligase, Ampligase, Tth ligase, T7 ligase, E. coli DNA ligase, 9° N™ DNA Ligase (NEB), or Taq Ligase) ligates the ends of the extension segment together with the next interposing barcode to produce a single integrated strand, as depicted in FIG. 2B. As non strand-displacing DNA polymerases have a slight ability to displace a DNA oligonucleotide from a template strand, the hybridization of the oligonucleotide can be enhanced in order to stop strand displacement by the polymerase. The DNA fragments are end repaired or end polished. Generally, a single adenine base is added to form an overhang via an A-tailing reaction. This “A” overhang allows adapters containing a single thymine overhanging base to base pair with the DNA fragments. Additional sequences such as universal adapters or primers may then be added using conventional means to permit platform specific sequences or to provide a binding site for sequencing primers, followed by fragmentation and additional library preparation steps according to commercial library prep kits.

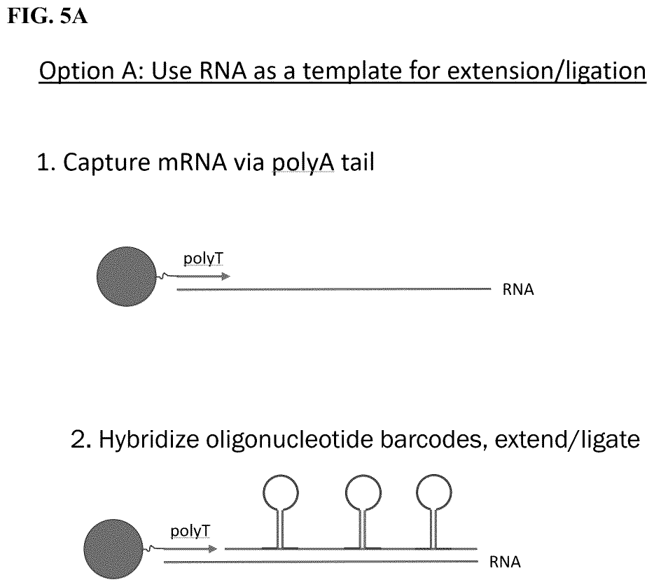

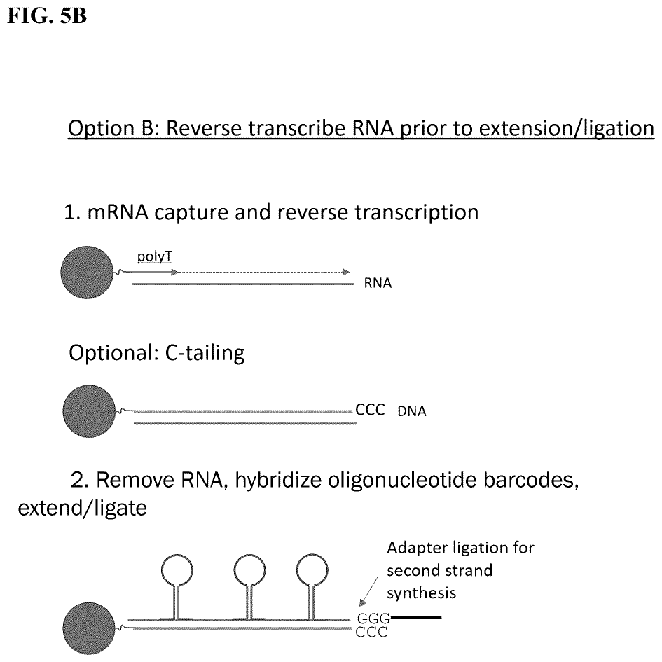

RNA Library Preparation is performed according to known methods described throughout the application and briefly below. One option, as depicted in FIG. 5A, RNA (e.g., mRNA) is captured by taking advantage of the poly-adenylated (poly(A)) tail. Briefly, a surface immobilized poly(T) (e.g., a bead containing a poly(T) sequence) hybridizes with the poly(A) portion of the input RNA. Sample interposing barcodes (as described herein) are added at an appropriate concentration such that there are approximately 50-200 bases between each hybridized adapter. A non strand-displacing polymerase extends the complement strand to generate an extension segment, as shown in FIG. 2A, and a ligase (e.g., T4 RNA ligase, T4 RNA Ligase 2, or PBCV-1 DNA Ligase) ligates the ends of the extension segment together with the next interposing barcode to produce a single integrated strand, as depicted in FIG. 2B. An alternative option, illustrated in FIG. 5B, a surface immobilized poly(T) (e.g., a bead containing a poly(T) sequence) hybridizes with the poly(A) portion of the input RNA. Also present, either before or after the poly(T) sequence, is a priming region for a reverse transcriptase. In the presence of a reverse transcriptase complementary DNA (cDNA) is generated. The cDNA may be optionally terminated with a plurality of cytosines, referred to as C-tailing in FIG. 5B. The RNA is then removed and sample interposing barcodes (as described herein) are added at an appropriate concentration such that there are approximately 50-200 bases between each hybridized adapter. A non strand-displacing polymerase extends the complement strand to generate an extension segment, as shown in FIG. 2A, and a ligase (e.g., T4 RNA ligase, T4 RNA Ligase 2, or PBCV-1 DNA Ligase) ligates the ends of the extension segment together with the next interposing barcode to produce a single integrated strand, as depicted in FIG. 2B.

As most wild-type reverse transcriptases have strong strand-displacement activity, the use of strand-displacement defective reverse transcriptases, in addition to modified oligonucleotides, is advantageous to embodiments of the methods herein. The combination of 5′ LNA-modified oligonucleotides (preferably up to 3 consecutive LNA's) and strand-displacement defective reverse transcriptase mutants such as Y64A M-MLV or F61W HIV have been described as leading to a near complete stop in strand-displacement. Various mutations in the HIV-2 reverse transcriptase have also been shown to abrogate strand-displacement activity (Martin-Alonso S et al. ACS Infect. Dis. 2020; 6(5):1140-53). Additional oligonucleotide modifications that may decrease strand-displacement include 2′ fluoro-nucleotides, PNAs, ZNAs, or G-clamps. DNA binding agents such as Actinomycin D have also been reported to reduce strand-displacement (see for example, U.S. Pat. No. 10,612,018).

The resultant integrated strand (i.e., the complementary strand containing a plurality of adapters) may be subjected to reaction conditions (e.g., elevated temperature or denaturing additives) such that the stem regions of interposing barcodes and/or any secondary structures present denature to form a linear integrated strand, as schematically shown in FIG. 2C. The integrated strand is then converted to double stranded DNA (dsDNA) using known techniques in the art (e.g., Single Strand Adapter Library Prep (SALP) or by ss-DNA ligation using a CircLigase™ (Lucigen)) and amplified according to the methods known in the art or described herein.

### Example 4: IBC-Led Reconstruction of Integrated Strand Long Reads

Nucleic acid preparation: Template regions to be sequenced (e.g. an integrated strand including a variable heavy (VH) and variable light (VL) chain, or an integrated strand including a TCRα/γ and TCRβ/δ chain) are amplified by PCR with a biotinylated primer and a non-biotinylated primer and a dNTP mix containing dUTP, dTTP, dATP, dGTP and dCTP. 0.25 pmols of template was pulled down using 100 μg of MyOne Streptavidin C1 (Invitrogen) beads in binding and wash buffer. The non-biotinylated strand of the template was then separated by denaturing with 0.1M NaOH.

Adapter annealing: Following template denaturation, the biotinylated strand-bound beads are washed twice with binding and wash buffer and resuspended in 1×T4 DNA ligase buffer in the presence of 0.5 mM total dNTPs and synthetic long read adapters at a final concentration of 150 nM each. The adapters are annealed onto the template by heating to 95° C. for three minutes and then cooling to 37° C. at 0.1° C./min and incubating at 37° C. for an additional 30 minutes. The slow rate of cooling ensures proper hybridization of the IBC to the target sequence.

Concatenation of adapters and synthetic strand isolation: Following adapter annealing, 1200 units of T4 DNA ligase (NEB) and 3 units of T4 DNA polymerase (NEB) are then added to the samples and samples incubated for a further 1 hour at 37° C. in order to produce the synthetic construct containing multiple IBCs. Beads are then pelleted, and the supernatant discarded. Beads are washed twice with 1× binding and wash buffer. The synthetic strand is then eluted by combining the beads with 20 μL of 0.1 M NaOH and incubating for 3 minutes and transferring 18 μL of the supernatant to a fresh tube containing 9 μL of 200 mM Tris, pH 8. The samples are treated with 1 U of Thermolabile User II enzyme (NEB) in the presence of 1× Cutsmart buffer (NEB) for 15 minutes and then purified with 1× volume sparQ beads (Quantabio).

Amplification and purification: 1 μL of the synthetic strand product is then amplified by PCR using primers that bind to the terminal adapters using Q5 or Phusion enzymes (NEB). PCR amplification is followed in real-time and stopped once the PCR reached the exponential phase. Samples are purified using sparQ beads and run on a 2% agarose gel. Products of appropriate size are then cut out and purified using the DNA agarose gel extraction kit (Zymo). 10,000 gel extracted molecules are used as template for a second round of PCR using the Q5 enzyme, with this PCR reaction also followed in real-time and stopped as soon as the reaction hit the exponential phase.

Library prep and sequencing: The 2^{nd} PCR reaction is then used as input to prepare sequencing library using the Quantabio DNA fragmentation and Library prep kit. Sequencing libraries are sequenced as 2×150 bp paired-end runs on a HiSeq X-10 sequencer (Illumina) to obtain 20 million reads (10 million clusters) per sample.

As depicted in FIG. 10, a non-limiting example of the assembly process is described. As described herein, a plurality of interposing barcodes (IBCs), are hybridized to a bridged polynucleotide, extended, and ligated together to form a tagged complement of the bridged polynucleotide. The IBCs are represented as single letters: A, B, C, D, E, and F in FIG. 10. The tagged complement was then amplified (step 2 of FIG. 10) and fragmented. The fragments are then sequenced, and the IBCs are identified for each sequencing read. The sequencing reads are grouped according to the co-occurrence of IBCs, (i.e., if UMI A is observed with B, and B is observed with C, A B and C must have all come from the same molecule). Inter-molecular chimeras can form during library prep, leading to UMIs from two distinct molecules being incorrectly associated. To resolve these errors, spurious UMI associations can be identified and filtered out based on their absolute frequency within the library (e.g., employing a filter that does not associate UMIs that are only observed together in a single read), or their relative frequency to other associations within the group (e.g., filter out UMI associations that are observed at <10 times the frequency of other neighboring UMI associations within a group). Given each processed UMI grouping, all the sequencing reads containing a group member are identified and assembled reconstruct the full-length target molecule. The regularly spaced UMI signatures in the aligned sequences are successful indicators of the reconstructed long read.

### Example 5. Interposing Probes for Targeted Sequencing

Described herein are methods for achieving greater read lengths by integrating specialized interposing probes, also referred to herein as probe inserts, into the target polynucleotide, in combination with the sequential targeted sequencing methods described herein. These methods enable sequencing of two, three, or more regions on a polynucleotide (e.g., the same single-stranded polynucleotide including interposing probes). Once sequencing of a first region is completed, for example, the sequenced strand may be removed (e.g., by digesting the strand or washing away with denaturing conditions). Alternatively, including a blocking element, such as modified nucleotides (e.g., terminating nucleotides), that is introduced following the sequencing of one region terminates extension on that specific strand. Subsequently, the next sequencing primer (e.g., a second, third, or fourth sequencing primer) is hybridized to another known region of the target nucleic acid (e.g., an endogenous sequence of the target nucleic acid, or an integrated probe sequence) and sequencing is re-initiated. Alternatively, the sequencing primers can be introduced in a single step and allowed to anneal to their respective region of interest. If all of the primers are loaded in a single step, the 3′ ends of the primers not being sequenced in the first series of sequencing cycles are non-extendable. Following sequencing and termination of the first sequenced strand, the next subsequent primer may be activated to initiate sequencing. Activating the primer may include removal of a blocking group on the primer (e.g., a blocking group with orthogonal removal conditions relative to the reversible terminators used during sequencing). The stepwise process of sequencing and terminating may also be utilized to facilitate long-range sequencing methods, for example, synthetic long reads, without the needs to remove intermediate sequencing products. These novel methods will, among other advantages, increase sequencing efficiency when targeting multiple regions on one or more template polynucleotides. In particular, these methods will provide for increase reagent savings, reduced labor costs, and more rapid sequencing turnaround times.

### Method: Interposing Probe Hybridization, Extension, and Ligation

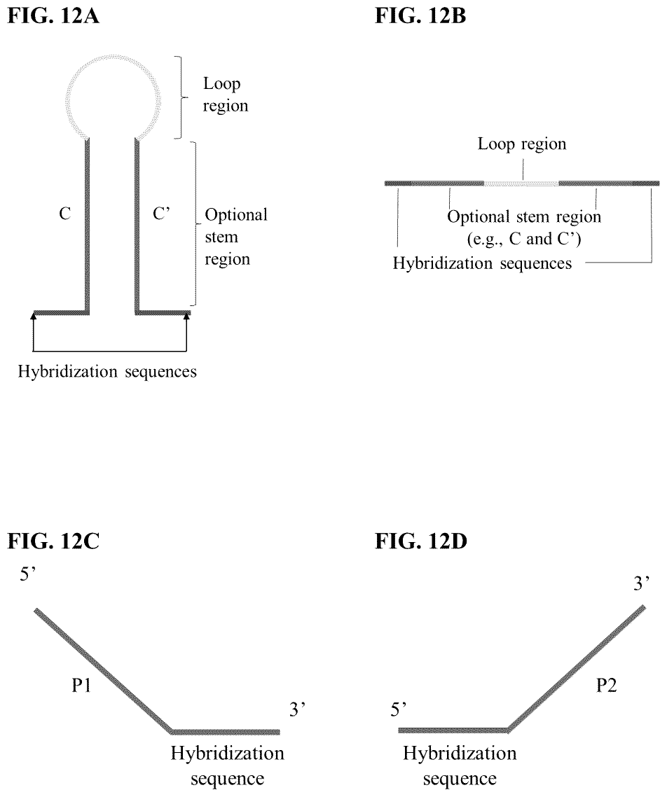

Briefly, FIG. 12A is an overview of a non-limiting example of an interposing probe (IPP), and includes a loop region, a stem region, and two hybridization sequences. The loop region may include about 10 to about 20 random nucleotides. In embodiments, the loop region includes between 5 to 15 T nucleotides (e.g., 5, 6, 7, 8, 9, 10, 11, 12, 13, 14, or 15 T nucleotides). Such random sequences may be referred to as molecular barcodes or unique molecular identifiers (UMI). In embodiments, the IPP includes a primer binding sequence. In embodiments, a primer binding sequence (e.g., a sequencing primer binding sequence) is located in the loop region. In embodiments of the methods described herein, synthetic long reads are constructed by aligning all sequencing reads that contain the same sequencing primer binding sequence. In embodiments, sequencing reads are additionally aligned based on the occurrence of the same UMI. Each sequencing primer binding sequence (or target/primer binding sequence combination) is unique, although rare multiple occurrences can be treated bioinformatically. The length of the sequencing primer binding sequence and/or UMI determines the number of target sequences and can be optimized for a given sequencing application. Aside from forming the backbone for long read alignment, the introduction of UMIs into sequencing libraries prior to target amplification by PCR has been shown to dramatically increase the sensitivity for rare mutations and enable absolute read counting. The stem region includes two known sequences capable of hybridizing to each other, ranging from about 5 to about 10 nucleotides, and is stable (i.e., capable to remaining hybridized together) at approximately a maximum temperature of 37° C., and unhybridizes (i.e., denatures) at temperatures greater than 50° C. Finally, the hybridization sequences each include about 3 to about 5 known nucleotides (e.g., AGTCG for pad 1, and GGGAG for pad 2) and are capable of hybridizing to single-stranded template nucleic acids (i.e., they are a complement to the original target). Hybridization sequence sequences may be designed such that each IPP hybridizes to the target polynucleotide at regularly spaced intervals, or they may be designed such that the IPPs hybridize to specific, non-adjacent target regions. FIG. 12B depicts the interposing barcode when the stem regions are denatured. Additionally, flanking adapters are included which may target either the 3′ end of the template DNA (see, FIG. 12C) or the 5′ end of the template DNA (see, FIG. 12D).

To an isolated DNA (e.g., an IgG HC+LC linked cDNA, sample interposing probes (as described herein) are added at an appropriate concentration such that there are approximately 50-100 bases between each IPP (see, FIG. 3). In some embodiments, each IBC hybridized on a polynucleotide includes a unique priming sequence on the 5′ end. A non strand-displacing polymerase (e.g., Klentaq, T4, T7, Bst, Phusion, Tfl, Pfu, or Stoffel fragment) extends the complement strand to generate an extension segment, and a ligase ligates the ends of the extension segment together with the next interposing barcode to produce a single integrated strand. The non strand-displacing polymerase can either be a naturally occurring enzyme, or one that is specifically engineered to minimize strand displacement.

As even “non-strand displacing” DNA polymerases can have a slight ability to displace a DNA oligonucleotide from a template strand of DNA, the hybridization of the oligonucleotide can be enhanced in order to stop strand displacement by the polymerase. Prevention of displacement can be achieved by using modifications to the oligonucleotide itself or by using additives that either stabilize the hybridization of the oligonucleotide or that stop the polymerase. Modifications to the oligonucleotides that reduce or inhibit the strand displacement activity of the polymerase are for instance 2′ fluoro nucleosides, PNAs, ZNAs, G-Clamps (U.S. Pat. No. 6,335,439, a cytosine analogue capable of clamp binding to guanine) or LNAs (US 2003/0092905; U.S. Pat. No. 7,084,125).

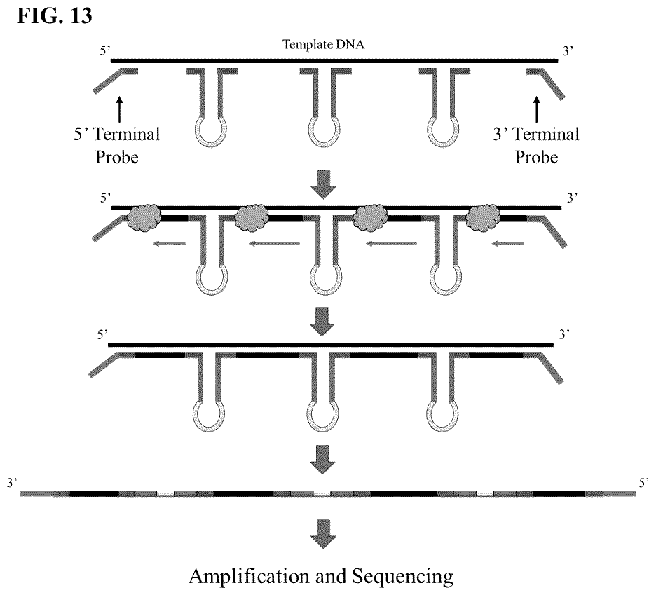

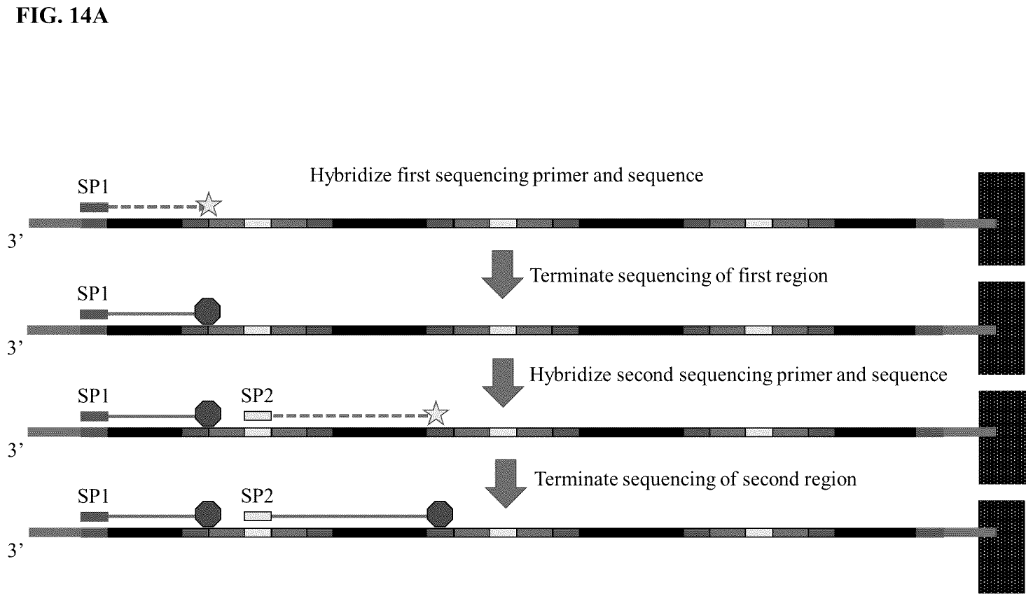

Optionally, the template DNA sample is washed away, and the resultant integrated strand may be subjected to reaction conditions (e.g., elevated temperature or denaturing additives) such that the stem regions of interposing barcodes and/or any secondary structures present denature to form a linear integrated strand, as schematically shown in FIG. 13. The integrated strand may be amplified using methods known to those skilled in the art (e.g., standard PCR amplification or rolling circle amplification) and subjected to standard library preparation methods as known to those skilled in the art and described herein. The integrated strand may serve as the input DNA with any commercially available library preparation kit. A variety of kits for making sequencing libraries from DNA are available commercially. Libraries may be prepared as described supra.

Library preparation (optional): Prior to sequencing, the input nucleic acid material may be fragmented using techniques known to those in the art. Three approaches available to fragment nucleic acid chains include: physical, enzymatic, and chemical. Nucleic acid fragmentation is typically done by physical methods (i.e., acoustic shearing and sonication) or enzymatic methods (i.e., non-specific endonuclease cocktails and transposase tagmentation reactions). Following fragmentation, the nucleic acid fragments are end-repaired or end-polished. Generally, a single adenine base is added to form an overhang via an A-tailing reaction. This “A” overhang allows adapters containing a single thymine overhanging base to base pair with the DNA fragments. Additional sequences such as adapters or primers may then be added using conventional means to permit platform specific sequences or to provide a binding site for sequencing primers.

Template amplification: Following construct formation, the constructs are amplified. The contents of an amplification reaction are known by one skilled in the art and include appropriate substrates (such as dNTPs), enzymes (e.g., a DNA polymerase) and buffer components required for an amplification reaction. Generally, amplification reactions require at least two amplification primers, often denoted ‘forward’ and ‘reverse’ primers (primer oligonucleotides) that are capable of annealing specifically to a part of the polynucleotide sequence to be amplified under conditions encountered in the primer annealing step of each cycle of an amplification reaction. In embodiments the forward and reverse primers include a sequence of nucleotides capable of annealing to a part of a primer-binding sequence in the polynucleotide molecule to be amplified (or the complement thereof if the template is viewed as a single strand) during the annealing step.

In embodiments, the amplification primers may be universal for all samples, or one of the forward or reverse primers may carry the tag sequence that codes for the sample source. The amplification primers may hybridize across the tag region of the ligated adapter, in which case unique primers will be needed for each sample nucleic acid. The amplification reaction may be performed with more than two amplification primers. In order to prevent the amplification of ligated adapter-adapter dimers, the amplification primers can be modified to contain nucleotides that hybridize across the whole of the ligated adapter and into the ligated template (or the dNTP's attached to the 3′ end thereof). This first amplification primer can be modified and treated to help prevent exonuclease digestion of the strands, and thus it may be advantageous to have a first amplification primer that is universal and can amplify all samples rather than modifying and treating each of the tagged primers separately. The tagged primer can be introduced as a sample specific third primer in the amplification reaction but does not need to be specially modified and treated.

The length of adapter sequence added to the 3′ and 5′ ends of each strand may be different. The amplification primers may also be of different lengths to each other and may hybridize to different lengths of the adapter, and therefore the length added to the ends of each strand can be controlled. The length of the added sequences may be 20-100 bases or more depending on the desired experimental design. The forward and reverse primers may be of sufficient length to hybridize to the whole of the adapter sequence and at least one base of the target sequence.

Primers may additionally include non-nucleotide chemical modifications, for example one or more phosphorothioate(s) to increase exonuclease resistance, again provided such that modifications do not prevent primer function. Modifications may, for example, facilitate attachment of the primer to a solid support, for example a biotin moiety. Certain modifications may themselves improve the function of the molecule as a primer, or may provide some other useful functionality, such as providing a site for cleavage to enable the primer (or an extended polynucleotide strand derived therefrom) to be cleaved from a solid support.

Amplification may also be performed using a first plurality of oligonucleotides and a second plurality of oligonucleotides, wherein one or more of the first plurality include a first sequence capable of hybridizing to a first endogenous region of a target polynucleotide, and wherein one or more of the second plurality include a second sequence capable of hybridizing to the complement of a second region of the target polynucleotide (see, U.S. Patent Application 63/311,576, which is incorporated herein by reference in its entirety). This method, for example has the advantage of not requiring extensive library prep or adapter ligation compared to existing commercial kit offerings, and allows for amplification and immobilization of an endogenous polynucleotide prior to extended-range targeted sequencing.

Sample immobilization: The term ‘solid-phase amplification’ as used herein refers to any nucleic acid amplification reaction carried out on or in association with a solid support such that all or a portion of the amplified products are immobilized on the solid support as they are formed. In particular, the term encompasses solid-phase polymerase chain reaction (solid-phase PCR) and solid phase isothermal amplification which are reactions analogous to standard solution phase amplification, except that one or both of the forward and reverse amplification primers is/are immobilized on the solid support. Although the invention encompasses ‘solid-phase’ amplification methods in which only one amplification primer is immobilized (the other primer usually being present in free solution), it is preferred for the solid support to be provided with both the forward and the reverse primers immobilized.

Nucleic acid molecules are hybridized in Tris HCl buffer with NaCl to a solid support (e.g., a flow cell) that contains forward and reverse nucleic acid primers. The library of nucleic acid molecules (approximately 1 pM concentration) is incubated for 15-30 min at 45° C. Surface amplification is carried out via any amplification method of choice (e.g., thermal cycling via PCR or isothermal eRCA). Alternatively, amplicons generated with biotin-labeled primers can be immobilized onto a solid support followed by denaturation to release the complementary strand. The monoclonal clusters can proceed to any necessary post-processing steps such as blocking of free 3′ ends, removal of select amplicons, or hybridization of a sequencing primer. Optionally, the clusters are quantified by introducing a nucleic acid stain (e.g., SYBR® Gold stain available from Thermo Fisher, Catalog #S11494 or a FAM (6-fluorescein amidite) labeled oligonucleotide) in the presence of a buffer is allowed to incubate with the amplicons for 10 minutes. After a wash, the substrate containing the stained amplicons is imaged and subjected to post-processing analysis to determine cluster size and brightness. After these steps, clusters are ready for sequencing in a sequencing-by-synthesis system.

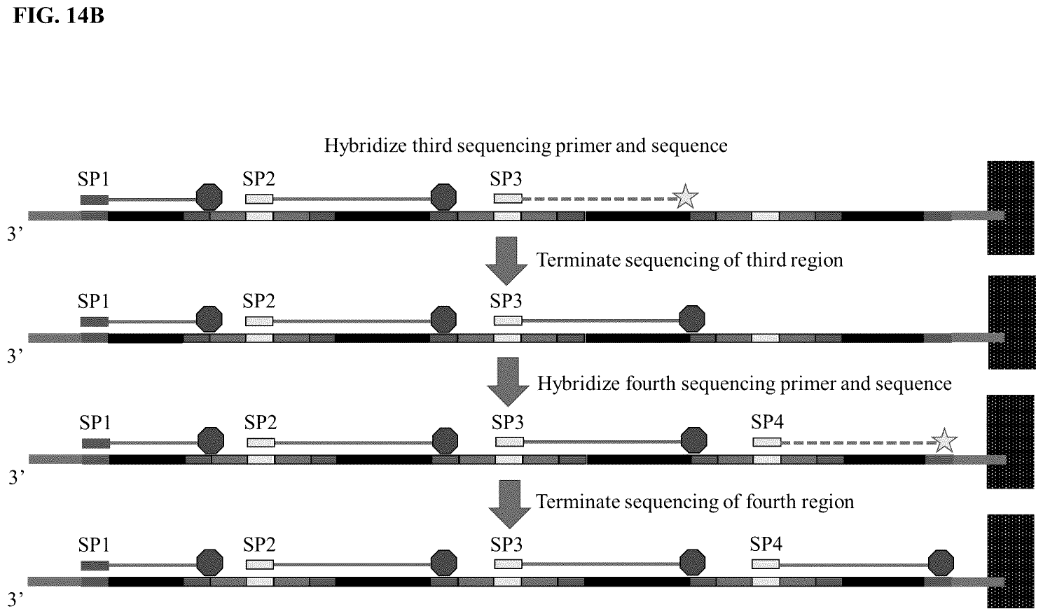

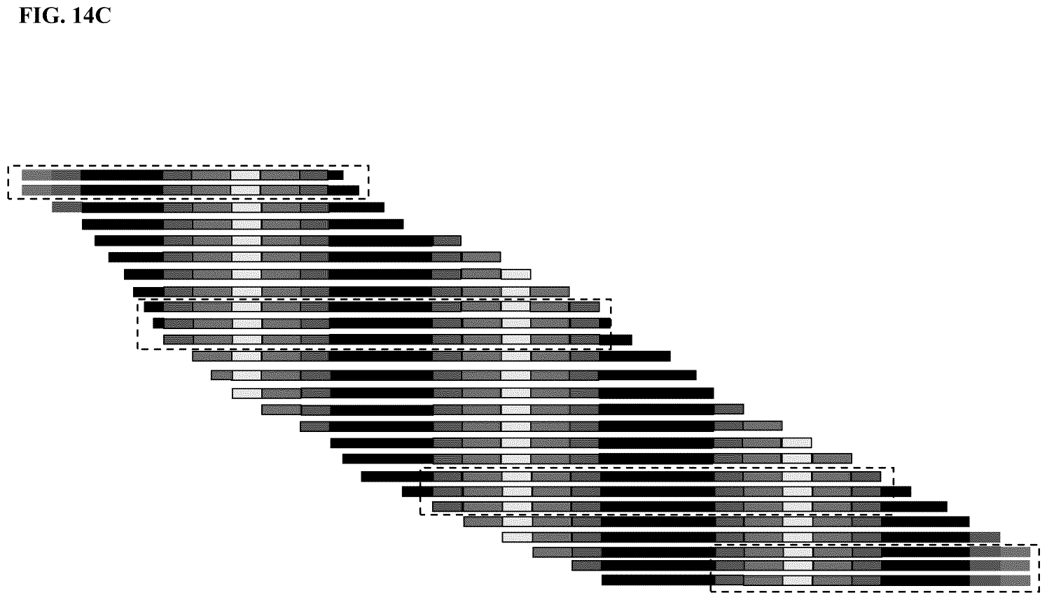

First region sequencing and termination: Sequencing is initiated by hybridizing a first sequencing primer to the polynucleotide template, and in the presence of a DNA polymerase and reversibly-terminated nucleotides, sequencing a first region of the polynucleotide. For example, to genomic DNA with known regions, two or more different primers are annealed to the known regions and sequenced in an iterative manner. FIG. 14A shows hybridization of a first sequencing primer (e.g., SP1) to the flanking adapter on the 3′ end of the DNA strand, wherein the template polynucleotide is immobilized to a solid support (illustrated as a dark rectangle), and sequencing a first region by extending the primer with a polymerase to incorporate and detect labeled nucleotides (depicted as a dashed line and star). As described herein, in embodiments, adapters are optional, and the first sequencing primer may be designed such that it targets an endogenous sequence of the target polynucleotide. Following sequencing of the first region, extension is terminated (e.g., by incorporating a ddNTP (shown here as an octagon), or by removing the sequenced strand).

Termination includes methods or reagents that preclude further sequencing of the first region while sequencing any subsequent regions of the template polynucleotide. For example, termination occurs by contacting the first region with a dideoxy nucleotide triphosphate (e.g., ddATP, ddTTP, ddCTP, ddGTP) or a combination thereof. The addition of a dideoxy nucleotide triphosphate which lacks a 3′-OH group required for the formation of a phosphodiester bond with an adjacent nucleotide, can inhibit further sequencing. In embodiments, termination includes the incorporated of unmodified dNTPs. In embodiments, a chain-terminating nucleotide includes any nucleotide or nucleotide analog that lacks a 3′-OH and is a substrate for a polymerase. For example, azidothymidine (AZT) is a chain-terminating nucleotide analog. Other chain-terminating nucleoside analogs are described, for example, in Yamamoto J et al. Molecules. 2016; 21(6):766, which is incorporated herein by reference in its entirety.

Alternatively, termination can include the introduction of reversibly-terminated nucleotides that are cleaved under different conditions than the modified nucleotides used when sequencing the second region. For example, terminating sequencing occurs by contacting the first region with reversibly-terminated nucleotides containing 3′-O-allyl group, which is cleaved by transition metal catalysis, a 3′-O-methoxymethyl group, which is cleaved by acid, a 3′-O-nitrobenzyl group, which is cleaved by light, or 3′-O—NH₂ group which is cleaved via buffered nitrous acid; and sequencing the second region uses RTs cleaved via a reducing agent (e.g., azidomethyl, or disulfide containing RTs). In some embodiments, terminating sequencing of the first region can include exhausting available template in the first sequencing read.

As an additional alternative, termination may include hybridizing an extension blocking primer (e.g., a blocker oligonucleotide) downstream of the sequenced strand, for example, such that additional labeled nucleotide may not be incorporated. Blocker oligonucleotides may include non-canonical nucleobase units and/or non-conventional linkages which result in the blocking oligonucleotide having high affinity for the targeted region (i.e., greater affinity for the targeted region than a correspondingly unmodified oligonucleotide). Non-canonical nucleobase units and/or non-conventional linkages suitable for incorporation into blocker oligonucleotides include, but are not limited to, LNAs and 2′-O-Me nucleobase units. Because of their high affinity for target nucleic acid, when contacted by the advancing portion of a polymerase (e.g., a DNA polymerase), blocking oligonucleotides halt strand extension by the polymerase. Typically, blocker oligonucleotides are non-extendible in the presence of a DNA polymerase, and thus are designed to prevent the initiation of DNA synthesis therefrom, e.g., by inclusion of a nucleobase unit at their 3′ end that prevents polymerase-based extension from the oligonucleotide. Additional extension blocking primers are described in U.S. Pat. Pub. US2014/0329282, which is incorporated herein by reference in its entirety.

Sequencing of additional target regions: Once sequencing of the first region is terminated, a second sequencing primer is then hybridized to a second known region of the polynucleotide. In some embodiments, the second sequenced region is located 5′ to the first sequenced region. In embodiments, the second sequenced region is adjacent to the first sequenced region. In embodiments, the second sequenced region is not adjacent to the first sequenced region. In some embodiments, following sequencing termination of the first region, the second primer is hybridized to the second region and is followed by sequencing of the second region. Next, a third sequencing primer (e.g., SP3) is hybridized to a known region of the polynucleotide (e.g., the loop portion of the first interposing probe) and the second region is sequenced. Sequencing of the second region is terminated, and then a third sequencing primer (e.g., SP3) is hybridized to a third region of the polynucleotide (e.g., the loop portion of the second interposing probe) and sequencing of the third region is performed, as illustrated in FIG. 14B. Once sequencing of the third region is complete, extension is terminated once again. Finally, a fourth sequencing primer (e.g., SP4) is hybridized to a known region of the polynucleotide (e.g., the loop portion of the third interposing probe) and the fourth region is sequenced. Sequencing of the fourth region is terminated. Although only four regions of the template polynucleotide as shown as being targeted by sequencing primers and sequenced, it will be understood that additional primers may be designed to target additional regions for sequencing. Any sequenced region may also contain an index sequence.

An alternate method for sequencing two or more regions on the same polynucleotide strand involves hybridizing a sequencing primer to the second target region (or third target region, fourth target region, etc.), extending in the presence of a polymerase and detectable nucleotides, and then switching to a sequencing reaction mixture with native nucleotides and performing a runoff extension (i.e., extending to a sufficient length). For example, runoff extension may be initiated once the index sequence in the second region has been completely acquired. Performing runoff extension is an alternative to terminating the extension, and reduces costs associated with performing sequencing cycles including modified nucleotides. Once extension of the second target region (or third target region, fourth target region, etc.) has been completed, a sequencing primer complementary to the first region is hybridized, and extension in the presence of a polymerase and detectable nucleotides is performed.

Sequencing can be carried out using any suitable sequencing-by-synthesis technique, wherein nucleotides are added successively to a free 3′ hydroxyl group, resulting in synthesis of a polynucleotide chain in the 5′ to 3′ direction. In embodiments, the identity of the nucleotide added is determined after each nucleotide addition. In some embodiments, terminating sequencing of the second region can include exhausting available template in the second sequencing read.

In embodiments, the sequencing method relies on the use of modified nucleotides that can act as reversible reaction terminators. Once the modified nucleotide has been incorporated into the growing polynucleotide chain complementary to the region of the template being sequenced there is no free 3′-OH group available to direct further sequence extension and therefore the polymerase cannot add further nucleotides. Once the identity of the base incorporated into the growing chain has been determined, the 3′ reversible terminator may be removed to allow addition of the next successive nucleotide. Such reactions can be done in a single experiment if each of the modified nucleotides has attached a different label, known to correspond to the particular base, to facilitate discrimination between the bases added at each incorporation step. Alternatively, a separate reaction may be carried out containing each of the modified nucleotides separately.

The modified nucleotides may carry a label (e.g., a fluorescent label) to facilitate their detection. Each nucleotide type may carry a different fluorescent label. However, the detectable label need not be a fluorescent label. Any label can be used which allows the detection of an incorporated nucleotide. One method for detecting fluorescently labeled nucleotides includes using laser light of a wavelength specific for the labeled nucleotides, or the use of other suitable sources of illumination. The fluorescence from the label on the nucleotide may be detected (e.g., by a CCD camera or other suitable detection means).

Use of the sequencing method outlined above is a non-limiting example, as essentially any sequencing methodology which relies on successive incorporation of nucleotides into a polynucleotide chain can be used. Suitable alternative techniques include, for example, pyrosequencing methods, FISSEQ (fluorescent in situ sequencing), MPSS (massively parallel signature sequencing), or sequencing by ligation-based methods.

### P-Embodiments

The present disclosure provides the following illustrative embodiments.

Embodiment P1. A method of amplifying a tagged complement of two independent single-stranded polynucleotides, said method comprising: i)isolating a cell comprising a plurality of polynucleotides; ii) generating a bridged polynucleotide comprising a sequence of a first independent polynucleotide of the cell linked to a sequence of a second independent polynucleotide of the cell or a complement thereof; iii) hybridizing to the bridged polynucleotide a plurality of interposing oligonucleotide barcodes; iv) extending the 3′ ends of the interposing oligonucleotide barcodes with one or more polymerases to create extension products; v) ligating adjacent ends of the extension products hybridized to the bridged polynucleotide thereby making an integrated strand tagged with a plurality of interposing oligonucleotide barcodes, wherein the integrated strand comprises sequences of the first and second independent polynucleotides or complements thereof; and vi) amplifying the integrated strand by an amplification reaction; wherein each of the interposing oligonucleotide barcodes comprises from 5′ to 3′: a. a first hybridization pad complementary to a first sequence of the bridged polynucleotide; b. a first stem region comprising a sequence common to the plurality of interposing oligonucleotide barcodes; c. a loop region comprising a barcode sequence, wherein the barcode sequence, alone or in combination with a sequence of one or both of (a) the bridged polynucleotide, or (b) one or more additional barcode sequences, uniquely distinguishes the bridged polynucleotide from bridged polynucleotides generated from other cells; d. a second stem region comprising a sequence complementary to the first stem region, wherein the second stem region is capable of hybridizing to the first stem region under hybridization conditions; and e. a second hybridization pad complementary to a second sequence of the bridged polynucleotide.

Embodiment P2. The method of Embodiment P1, further comprising sequencing the amplified integrated strand.

Embodiment P3. The method of Embodiment P2, wherein the sequencing comprises: (A) fragmenting the amplified products of step (vi) to produce fragments, (B) ligating adapters to the fragments, (C) amplifying the resultant products from step (B) to generate amplicons, and (D) performing a sequencing reaction on the amplicons from step (C).

Embodiment P4. The method of Embodiment P2, wherein the sequencing further comprises (a) producing a plurality of sequencing reads; (b) grouping sequencing reads based on co-occurrence of barcode sequences; and (c) within each group, aligning the reads that belong to the same strand of an original sample polynucleotide based on the sequences of the barcode sequences.

Embodiment P5. The method of Embodiment P2, wherein the sequencing comprises sequencing by synthesis, sequencing by ligation, or pyrosequencing.

Embodiment P6. The method of Embodiment P4, wherein each of the sequencing reads comprise at least a portion of two or more barcode sequences, or complements thereof.

Embodiment P7. The method of Embodiment P4, wherein aligning the reads comprises alignment to a reference genome.

Embodiment P8. The method of Embodiment P4, further comprising forming a consensus sequence for reads having the same barcode sequence.

Embodiment P9. The method of Embodiment P4, further comprising computationally reconstructing sequences of a plurality of individual strands of original sample polynucleotides by removing interposing oligonucleotide barcode-derived sequences and joining sequences for adjacent portions of the sample polynucleotide.

Embodiment P10. The method of Embodiment P9, further comprising forming a consensus sequence for reads having the same barcode sequence.

Embodiment P11. The method of Embodiment P1, wherein generating the bridged polynucleotide comprises hybridizing a bridge oligonucleotide to the first independent polynucleotide and the second independent polynucleotide, wherein the bridge oligonucleotide comprises from 5′ to 3′ a first hybridization pad complementary to the first independent polynucleotide, a linking polynucleotide sequence, and a second hybridization pad complementary to the second independent polynucleotide.

Embodiment P12. The method of Embodiment P11, wherein the bridge oligonucleotide comprises one or more barcode sequences.

Embodiment P13. The method of Embodiment P1, wherein generating the bridged polynucleotide comprises overlap-extension PCR (OE PCR).

Embodiment P14. The method of Embodiment P1, wherein generating the bridged polynucleotide comprises hybridizing a bridge oligonucleotide to a 3′ end of the first independent polynucleotide and a 5′ end of the second independent polynucleotide.

Embodiment P15. The method of Embodiment P1, wherein generating the bridged polynucleotide comprises linking a sequence from a 5′ end of the first independent polynucleotide to a complement of a sequence from a 5′ end of the second independent polynucleotide.

Embodiment P16. The method of Embodiment P1, wherein each of the interposing oligonucleotide barcodes comprise a phosphorylated 5′ end.

Embodiment P17. The method of Embodiment P1, wherein the method comprises phosphorylating the 5′ ends of the interposing oligonucleotide barcodes prior to step (v).

Embodiment P18. The method of Embodiment P1, wherein each hybridization pad comprises about 9 to about 15 nucleotides.

Embodiment P19. The method of Embodiment P1, wherein each hybridization pad comprises about 8 to about 12 nucleotides.

Embodiment P20. The method of Embodiment P1, wherein each hybridization pad comprises a targeted primer sequence.

Embodiment P21. The method of Embodiment P1, wherein each hybridization pad comprises at least one locked nucleic acid.

Embodiment P22. The method of Embodiment P1, wherein the total combined length of the first hybridization pad and the second hybridization pad comprises about 18 to about 25 nucleotides.

Embodiment P23. The method of Embodiment P1, wherein the first and second stem regions are complementary and wherein each stem region comprises a known sequence of about 5 to about 10 nucleotides.

Embodiment P24. The method of Embodiment P1, wherein the first and second stem regions are complementary and wherein each stem region comprises a known sequence of about 6 to about 8 nucleotides.

Embodiment P25. The method of Embodiment P1, wherein the loop region comprises about 5 to about 20 nucleotides, or about 10 to about 20 nucleotides.

Embodiment P26. The method of Embodiment P1, wherein the loop region comprises about 12 to about 16 nucleotides.

Embodiment P27. The method of Embodiment P1, wherein each barcode sequence is selected from a set of barcode sequences represented by a random or partially random sequence.

Embodiment P28. The method of Embodiment P1, wherein each barcode sequence is selected from a set of barcode sequences represented by a random sequence.

Embodiment P29. The method of Embodiment P1, wherein the loop region further comprises a sample index sequence.

Embodiment P30. The method of Embodiment P1, wherein each barcode sequence differs from every other barcode sequence by at least two nucleotide positions.

Embodiment P31. The method of Embodiment P1, wherein each of the two independent single-stranded polynucleotides comprise a gene or gene fragment.

Embodiment P32. The method of Embodiment P31, wherein the gene or gene fragment is a cancer-associated gene or fragment thereof, T cell receptor (TCRs) gene or fragment thereof, or a B cell receptor (BCRs) gene, or fragment thereof.

Embodiment P33. The method of Embodiment P31, wherein the gene or gene fragment is a CDR3 gene or fragment thereof, T cell receptor alpha variable (TRAV) gene or fragment thereof, T cell receptor alpha joining (TRAJ) gene or fragment thereof, T cell receptor alpha constant (TRAC) gene or fragment thereof, T cell receptor beta variable (TRBV) gene or fragment thereof, T cell receptor beta diversity (TRBD) gene or fragment thereof, T cell receptor beta joining (TRBJ) gene or fragment thereof, T cell receptor beta constant (TRBC) gene or fragment thereof, T cell receptor gamma variable (TRGV) gene or fragment thereof, T cell receptor gamma joining (TRGJ) gene or fragment thereof, T cell receptor gamma constant (TRGC) gene or fragment thereof, T cell receptor delta variable (TRDV) gene or fragment thereof, T cell receptor delta diversity (TRDD) gene or fragment thereof, T cell receptor delta joining (TRDJ) gene or fragment thereof, or T cell receptor delta constant (TRDC) gene or fragment thereof.

Embodiment P34. The method of Embodiment P1, wherein each of the two independent single-stranded polynucleotides comprise messenger RNA (mRNA).

Embodiment P35. The method of Embodiment P1, further comprising hybridizing to the bridged polynucleotide a terminal adapter, wherein the terminal adapter comprises a first hybridization pad complementary to a first sequence of the bridged polynucleotide, a barcode sequence, and a primer binding sequence.

Embodiment P36. The method of Embodiment P35, wherein amplifying comprises hybridizing an amplification primer to the primer binding sequence of the terminal adapter and cycles of primer extension with a polymerase and nucleotides to generate amplified products.

Embodiment P37. The method of Embodiment P35, wherein the amplification reaction comprises polymerase chain reaction (PCR), strand displacement amplification (SDA), multiple displacement amplification (MDA), ligation chain reaction, transcription mediated amplification (TMA), nucleic acid sequence-based amplification (NASBA), rolling circle amplification (RCA), exponential rolling circle amplification (eRCA), hyperbranched rolling circle amplification (HRCA), or a combination thereof.

Embodiment P38. A bridged polynucleotide comprising a complement of a first independent single-stranded polynucleotide, a bridging oligonucleotide, a complement of a second independent single-stranded polynucleotide, and a plurality of interposing oligonucleotide barcode adapters.

Embodiment P39. A kit comprising the bridged polynucleotide and the plurality of interposing oligonucleotide adapters of Embodiment P38.

### ADDITIONAL EMBODIMENTS

The present disclosure provides the following additional illustrative embodiments.

Embodiment 1. A method of amplifying a tagged complement of two independent single-stranded polynucleotides, said method comprising: a. hybridizing a bridge oligonucleotide to a first polynucleotide and a second polynucleotide, thereby forming a bridged polynucleotide complex; b. hybridizing one or more interposing oligonucleotide probes to the first polynucleotide and second polynucleotide, wherein each of the interposing oligonucleotide probes comprises from 5′ to 3′: i. a first hybridization sequence complementary to a first sequence of said first polynucleotide and second polynucleotide; ii. a loop region comprising a primer binding sequence and optionally a barcode; and iii. a second hybridization sequence complementary to a second sequence of first polynucleotide and second polynucleotide; c. extending the 3′ end of each second hybridization sequence of said interposing oligonucleotide probes and the 3′ end of the hybridization sequence of said bridge oligonucleotide with one or more polymerases thereby forming an extension product of each of said oligonucleotide probes; d. ligating the 3′ end of each of said extension products to the 5′ end of the adjacent extension products, thereby making an integrated strand comprising a complement of the template nucleic acid comprising a plurality of the oligonucleotide probes; and e. amplifying the integrated strand by an amplification reaction to produce a tagged complement of two independent single-stranded polynucleotides.

Embodiment 2. The method of Embodiment 1, wherein the bridge oligonucleotide comprises, from 5′ to 3′, a first hybridization sequence complementary to a 3′ sequence of the first independent polynucleotide, and a second hybridization sequence complementary to a 5′ sequence of the second independent polynucleotide.

Embodiment 3. The method of Embodiment 1 or 2, further comprising hybridizing a 5′ terminal oligonucleotide probe downstream of the one or more interposing oligonucleotide probes to the first polynucleotide, and hybridizing a 3′ terminal oligonucleotide probe upstream of the one or more interposing oligonucleotide probes to the second polynucleotide; wherein the 5′ terminal oligonucleotide probe comprises from 5′ to 3′: i. a hybridization sequence complementary to a sequence of said first polynucleotide; and ii. a primer binding sequence; and wherein the 3′ terminal oligonucleotide probe comprises from 3′ to 5′: i. a hybridization sequence complementary to a sequence of said second polynucleotide; and ii. a primer binding sequence.

Embodiment 4. The method of Embodiment 3, further comprising extending the 3′ end of the hybridization sequence of said 3′ terminal oligonucleotide probe with one or more polymerases thereby forming an extension product.

Embodiment 5. The method of Embodiment 3 or 4, further comprising ligating the 5′ end of the 5′ terminal oligonucleotide probe to the 3′ end of the adjacent extension product.

Embodiment 6. The method of any one of Embodiments 1 to 5, wherein the bridge oligonucleotide comprises from 5′ to 3′: i. a first hybridization sequence complementary to a 3′ terminal sequence of the first independent polynucleotide; ii. a linker sequence; and iii. a second hybridization sequence complementary to a 5′ terminal sequence of the second independent polynucleotide.

Embodiment 7. The method of any one of Embodiments 1 to 6, wherein the 5′ end of the bridge oligonucleotide comprises about 5 to about 50 nucleotides complementary to the 3′ end of the first independent polynucleotide, and wherein the 3′ end of the bridge oligonucleotide comprises about 5 to about 50 nucleotides complementary to the 5′ end of the second independent polynucleotide.

Embodiment 8. A method of amplifying a tagged complement of two independent single-stranded polynucleotides, said method comprising: i) hybridizing a first overlap oligonucleotide to the first independent polynucleotide and a second overlap oligonucleotide to the second independent polynucleotide, and extending both the first and second overlap oligonucleotides with a polymerase, thereby forming an overlapped polynucleotide complex, wherein the overlapped polynucleotide complex comprises a complement of the first independent polynucleotide, the first overlap oligonucleotide, the second overlap oligonucleotide, and a complement of the second independent polynucleotide, wherein a 5′ sequence of the first overlap oligonucleotide is hybridized to a 5′ sequence of the second overlap oligonucleotide; ii) linking said overlapped polynucleotide complex, thereby generating a bridged polynucleotide comprising a sequence of the first independent polynucleotide linked to a sequence of the second independent polynucleotide or a complement thereof, iii) hybridizing to the bridged polynucleotide one or more interposing oligonucleotide barcodes, wherein each of the interposing oligonucleotide barcodes comprises from 5′ to 3′: i. a first hybridization sequence complementary to a first sequence of said bridged polynucleotide; ii. a loop region comprising a barcode; and iii. a second hybridization sequence complementary to a second sequence of said bridged polynucleotide; iv) extending the 3′ end of each second hybridization sequence of said interposing oligonucleotide barcodes with one or more polymerases thereby forming an extension product of each of said interposing oligonucleotide barcodes; v) ligating the 3′ end of each of said extension products to the 5′ end of the adjacent extension products hybridized to the bridged polynucleotide thereby making an integrated strand tagged with a plurality of interposing oligonucleotide barcodes, wherein the integrated strand comprises sequences of the first and second independent polynucleotides or complements thereof, and vi) amplifying the integrated strand by an amplification reaction to produce a tagged complement of two independent single-stranded polynucleotides.

Embodiment 9. The method of Embodiment 8, wherein each interposing oligonucleotide barcode comprises a first stem region comprising a sequence common to the plurality of interposing oligonucleotide barcodes and a second stem region comprising a sequence complementary to the first stem region, wherein the second stem region is capable of hybridizing to the first stem region under hybridization conditions.

Embodiment 10. The method of Embodiment 8 or 9, wherein the first overlap oligonucleotide comprises from 5′ to 3′ a first hybridization sequence complementary to a 5′ sequence of the second overlap oligonucleotide, and a second hybridization sequence complementary to a 3′ sequence of the first independent polynucleotide, and wherein the second overlap oligonucleotide comprises from 5′ to 3′ a first hybridization sequence complementary to a 5′ sequence of the first overlap oligonucleotide and a second hybridization sequence complementary to a 5′ sequence of the second independent polynucleotide.

Embodiment 11. The method of any one of Embodiments 8 to 10, wherein prior to step (i), the method further comprises isolating a cell comprising a plurality of polynucleotides, wherein the plurality of polynucleotides comprises the first independent polynucleotide and the second independent polynucleotide.

Embodiment 12. The method of any one of Embodiments 8 to 11, wherein the barcode sequence of each interposing oligonucleotide barcode, alone or in combination with a sequence of one or both of (a) the bridged polynucleotide, or (b) one or more additional barcode sequences, distinguishes the bridged polynucleotide from bridged polynucleotides generated from other cells.

Embodiment 13. The method of any one of Embodiments 1 to 12, further comprising sequencing the amplified integrated strand.

Embodiment 14. The method of Embodiment 13, wherein the sequencing comprises: (A) fragmenting the amplified products of step (vi) to produce fragments, (B) ligating adapters to the fragments, (C) amplifying the resultant products from step (B) to generate amplicons, and (D) performing a sequencing reaction on the amplicons from step (C).

Embodiment 15. The method of Embodiment 13 or 14, wherein the sequencing comprises sequencing by synthesis, sequencing by binding, sequencing by ligation, or pyrosequencing.

Embodiment 16. The method of any one of Embodiments 8 to 15, wherein each of the interposing oligonucleotide barcodes comprise a phosphorylated 5′ end.

Embodiment 17. The method of any one of Embodiments 8 to 15, wherein the method comprises phosphorylating the 5′ ends of the interposing oligonucleotide probes barcodes prior to step (v).

Embodiment 18. The method of any one of Embodiments 8 to 17, further comprising hybridizing to the bridged polynucleotide a terminal adapter, wherein the terminal adapter comprises a first hybridization sequence complementary to a first sequence of the bridged polynucleotide, a barcode sequence, and a primer binding sequence.

Embodiment 19. The method of Embodiment 18, wherein amplifying comprises hybridizing an amplification primer to the primer binding sequence of the terminal adapter and cycles of primer extension with a polymerase and nucleotides to generate amplified products.

Embodiment 20. The method of any one of Embodiments 8 to 19, wherein the amplification reaction comprises polymerase chain reaction (PCR), strand displacement amplification (SDA), multiple displacement amplification (MDA), ligation chain reaction, transcription mediated amplification (TMA), nucleic acid sequence-based amplification (NASBA), rolling circle amplification (RCA), exponential rolling circle amplification (eRCA), hyperbranched rolling circle amplification (HRCA), or a combination thereof.

Embodiment 21. A bridged polynucleotide comprising a complement of a first independent single-stranded polynucleotide sequence, a bridging oligonucleotide sequence, a complement of a second independent single-stranded polynucleotide sequence, and a plurality of interposing oligonucleotide barcode adapters.

Embodiment 22. A method of forming an integrated strand complement of a bridged polynucleotide comprising a plurality of oligonucleotide probes, wherein the bridged polynucleotide comprises a complement of two independent single-stranded polynucleotides, the method comprising: a. hybridizing a bridge oligonucleotide to a first independent polynucleotide and a second independent polynucleotide, thereby forming a bridged polynucleotide complex; b. amplifying said bridged polynucleotide complex, thereby generating a bridged polynucleotide comprising a sequence of the first independent polynucleotide linked to a sequence of the second independent polynucleotide, or a complement thereof, c. hybridizing one or more interposing oligonucleotide probes to the bridged polynucleotide, hybridizing a 5′ terminal oligonucleotide probe downstream of the one or more interposing oligonucleotide probes, and hybridizing a 3′ terminal oligonucleotide probe upstream of the one or more interposing oligonucleotide probes, wherein each of the interposing oligonucleotide probes comprises from 5′ to 3′: i. a first hybridization sequence complementary to a first sequence of said bridged polynucleotide; ii. a loop region comprising a primer binding sequence; and iii. a second hybridization sequence complementary to a second sequence of said bridged polynucleotide; wherein the 5′ terminal oligonucleotide probe comprises from 5′ to 3′: i. a hybridization sequence complementary to a third sequence of said bridged polynucleotide; and ii. a primer binding sequence; and wherein the 3′ terminal oligonucleotide probe comprises from 3′ to 5′: i. a hybridization sequence complementary to a fourth sequence of said bridged polynucleotide; and ii. a primer binding sequence; d. extending the 3′ end of each second hybridization sequence of said interposing oligonucleotide probes and the 3′ end of the hybridization sequence of said 3′ terminal oligonucleotide probe with one or more polymerases thereby forming an extension product of each of said oligonucleotide probes; e. ligating the 3′ end of each of said extension products to the 5′ end of the adjacent extension products, and ligating the 5′ end of the 5′ terminal oligonucleotide probe to the 3′ end of the adjacent extension product, each hybridized to the same bridged polynucleotide thereby making an integrated strand comprising a complement of the bridged polynucleotide comprising a plurality of the oligonucleotide probes; and f. amplifying the integrated strand by an amplification reaction to produce a complement of the integrated strand thereby forming an integrated strand complement of the bridged polynucleotide comprising oligonucleotide probes, wherein the complement of the integrated strand comprises a complement of the plurality of oligonucleotide probes.

Embodiment 23. The method of Embodiment 22, wherein the bridge oligonucleotide comprises, from 5′ to 3′, a first hybridization sequence complementary to a 3′ sequence of the first independent polynucleotide, and a second hybridization sequence complementary to a 5′ sequence of the second independent polynucleotide.

Embodiment 24. The method of Embodiment 22 or 23, wherein prior to step (a), the method further comprises isolating a cell comprising a plurality of polynucleotides, wherein said plurality of polynucleotides comprises the first independent polynucleotide and the second independent polynucleotide.

Embodiment 25. The method of any one of Embodiments 22 to 24, further comprising sequencing the amplified product of step (f).

Embodiment 26. The method of Embodiment 25, wherein the sequencing further comprises (a) producing a plurality of sequencing reads; (b) grouping sequencing reads based on co-occurrence of interposing oligonucleotide probe sequences; and (c) within each group, aligning the sequencing reads that belong to the same strand of an original bridged polynucleotide based on the sequences of the interposing oligonucleotide probe sequences.

Embodiment 27. The method of Embodiment 25 or 26, wherein prior to sequencing, the method further comprises hybridizing a sequencing primer to the primer binding sequence of one of the plurality of interposing oligonucleotide probes in the integrated strands.

Embodiment 28. The method of Embodiment 25 or 26, wherein prior to sequencing, the method further comprises hybridizing a sequencing primer to the primer binding sequence of the integrated oligonucleotide probe.

Embodiment 29. The method of any one of Embodiments 22 to 28, wherein the 5′ terminal oligonucleotide probe comprises from 5′ to 3′: i. a first hybridization sequence complementary to a first 5′ terminal sequence of the bridged polynucleotide; ii. a loop region comprising a primer binding sequence; and iii. a second hybridization sequence complementary to a second 5′ terminal sequence of the bridged polynucleotide, wherein said first and second 5′ terminal sequences are upstream of the bridged polynucleotide sequence complementary to the interposing oligonucleotide probes.

Embodiment 30. The method of any one of Embodiments 22 to 29, the 3′ terminal oligonucleotide probe comprises from 3′ to 5′: i. a first hybridization sequence complementary to a first 3′ terminal sequence of the bridged polynucleotide; ii. a loop region comprising a primer binding sequence; and iii. a second hybridization sequence complementary to a second 3′ terminal sequence of the bridged polynucleotide, wherein said first and second 3′ terminal sequences are downstream of the bridged polynucleotide sequence complementary to the interposing oligonucleotide probes.

Embodiment 31. The method of any one of Embodiments 22 to 31, wherein the first hybridization sequence, the second hybridization sequence, and the primer binding sequence is different between each interposing oligonucleotide probe of a plurality of interposing oligonucleotide probe.

Embodiment 32. A method of sequencing at least three regions of the integrated strand complement of the bridged polynucleotide comprising oligonucleotide probes of Embodiments 1 or 22, the method comprising: (a) contacting a first primer annealed to a first region of the integrated strand complement with a sequencing solution comprising a plurality of nucleotides and incorporating with a polymerase one or more nucleotides into the first primer to create a first extension strand, and detecting the one or more incorporated nucleotides so as to identify each incorporated nucleotide in said first extension strand; (b) contacting the integrated strand complement with a blocking element thereby terminating extension of the first extension strand thereby forming a blocked first extension strand; (c) contacting a second primer annealed to a second region of the integrated strand complement with a sequencing solution comprising a plurality of nucleotides and incorporating with a polymerase one or more nucleotides into the second primer to create a second extension strand, and detecting the one or more incorporated nucleotides so as to identify each incorporated nucleotide in said second extension strand; (d) contacting the integrated strand complement with a blocking element thereby terminating extension of the second extension strand and creating a blocked second extension strand; and (e) contacting a third primer annealed to a third region of the integrated strand complement with a sequencing solution comprising a plurality of nucleotides and incorporating with a polymerase one or more nucleotides into the third primer to create a third extension strand, and detecting the one or more incorporated nucleotides so as to identify each incorporated nucleotide in said third extension strand.

Embodiment 33. The method of Embodiment 32, wherein the blocked first extension strand is upstream of the blocked second extension strand, third extension strand, or both the blocked second extension strand and third extension strand.

Embodiment 34. The method of Embodiment 32 or 33, wherein the blocking element comprises a chain-terminating nucleotide.

Embodiment 35. The method of Embodiment 34, wherein said chain-terminating nucleotide comprises a ddNTP, a reversibly-terminated dNTP, or a modified nucleotide triphosphate which lacks a 3′-OH.

Embodiment 36. The method of any one of Embodiments 32 to 35, wherein contacting the integrated strand complement with a blocking element comprises hybridizing a blocking oligonucleotide downstream of the extension strand.

Embodiment 37. The method of Embodiment 36, wherein said blocking oligonucleotide comprises locked nucleic acids (LNAs), Bis-locked nucleic acids (bisLNAs), twisted intercalating nucleic acids (TINAs), bridged nucleic acids (BNAs), 2′-O-methyl RNA:DNA chimeric nucleic acids, minor groove binder (MGB) nucleic acids, morpholino nucleic acids, C5-modified pyrimidine nucleic acids, peptide nucleic acids (PNAs), phosphorothioate nucleic acids, or combinations thereof.

Embodiment 38. The method of Embodiment 37, wherein said blocking oligonucleotide inhibits nucleotide incorporation.

Embodiment 39. The method of any one of Embodiments 32 to 38, wherein the 3′ end of one or more of the extension strands is capable of ligating to the 5′ end of one or more different extension strands.

Embodiment 40. The method of any one of Embodiments 32 to 39, further comprising contacting the integrated strand complement with a blocking element thereby terminating extension of the third extension strand thereby forming a blocked third extension strand.

Embodiment 41. The method of Embodiment 40, further comprising contacting a fourth primer annealed to a fourth region of the integrated strand complement and incorporating one or more nucleotides into the fourth primer with a polymerase to create a fourth extension strand, and detecting the one or more incorporated nucleotides so as to identify each incorporated nucleotide in said fourth extension strand.

Embodiment 42. The method of any one of Embodiments 32 to 41, wherein between 4 to 9 regions or 9 to 15 regions of the integrated strand complement are sequenced.

Embodiment 43. The method of any one of Embodiments 32 to 41, wherein between 15 to 30 regions or 30 to 50 regions of the integrated strand complement are sequenced.

Embodiment 44. The method of any one of Embodiments 1 to 43, wherein the bridge oligonucleotide comprises one or more barcode sequences.

Embodiment 45. The method of any one of Embodiments 8 to 20, wherein amplifying the overlapped polynucleotide complex comprises overlap-extension PCR (OE PCR).

Embodiment 46. The method of any one of Embodiments 1 to 7 or 22 to 43, wherein step a) comprises hybridizing a bridge oligonucleotide to a 3′ end of the first independent polynucleotide and a 5′ end of the second independent polynucleotide.

Embodiment 47. The method of any one of Embodiments 1 to 7 or 22 to 43, wherein step a) comprises linking a sequence from a 5′ end of the first independent polynucleotide to a complement of a sequence from a 5′ end of the second independent polynucleotide.

Embodiment 48. A bridged polynucleotide comprising a complement of a first independent single-stranded polynucleotide sequence, a bridging oligonucleotide sequence, a complement of a second independent single-stranded polynucleotide sequence, and a plurality of interposing oligonucleotide probes.

Embodiment 49. A kit comprising the bridging polynucleotide and the plurality of interposing oligonucleotide probes of Embodiment 48.

Embodiment 50. A kit comprising: (a) a plurality of interposing oligonucleotide probes capable of hybridizing to a bridged polynucleotide, said interposing oligonucleotide probes comprising from 5′ to 3′: i. a first hybridization sequence complementary to a first sequence of the bridged polynucleotide; ii. a loop region comprising a primer binding sequence; and iii. a second hybridization sequence complementary to a second sequence of the bridged polynucleotide; (b) a plurality of 5′ terminal oligonucleotide probes capable of hybridizing to a bridged polynucleotide, said 5′ terminal oligonucleotide probes comprising from 5′ to 3′: i. a hybridization sequence complementary to a 5′ terminal sequence of the bridged polynucleotide, wherein the 5′ terminal sequence is upstream of the bridged polynucleotide sequence complementary to the interposing oligonucleotide probes; and ii. a primer binding sequence; (c) a plurality of 3′ terminal oligonucleotide probes capable of hybridizing to a bridged polynucleotide, said 3′ terminal oligonucleotide probes comprising from 3′ to 5′: i. a hybridization sequence complementary to a 3′ terminal sequence of the bridged polynucleotide, wherein the 3′ terminal sequence is downstream of the bridged polynucleotide sequence complementary to the interposing oligonucleotide probes; and ii. a primer binding sequence; and (d) a bridging oligonucleotide comprising from 5′ to 3′: i. a hybridization sequence complementary to a 3′ terminal sequence of a first independent polynucleotide; ii. a linker sequence; and iii. a hybridization sequence complementary to a 5′ terminal sequence of a second independent polynucleotide.

1. A method of amplifying a tagged complement of two independent single-stranded polynucleotides, said method comprising:
a. hybridizing a bridge oligonucleotide to a first independent polynucleotide and a second independent polynucleotide, thereby forming a bridged polynucleotide complex;
b. hybridizing a first interposing oligonucleotide probe to the first independent polynucleotide and hybridizing a second interposing oligonucleotide probe to the second independent polynucleotide, wherein each interposing oligonucleotide probe comprises, from 5′ to 3′:
i. a first hybridization sequence;
ii. a loop region comprising a primer binding sequence and optionally a barcode; and
iii. a second hybridization sequence;
c. extending the 3′ end of each second hybridization sequence of said interposing oligonucleotide probes and a 3′ end of said bridge oligonucleotide with a polymerase thereby forming extension products;
d. ligating the 3′ end of each of said extension products to the 5′ end of the adjacent extension products, thereby making an integrated strand; and
e. amplifying the integrated strand by an amplification reaction to produce a tagged complement of two independent single-stranded polynucleotides.

2. The method of claim 1, wherein the bridge oligonucleotide comprises, from 3′ to 5′, a first hybridization sequence complementary to a 3′ sequence of the first independent polynucleotide, and a second hybridization sequence complementary to a 5′ sequence of the second independent polynucleotide.

3. The method of claim 1, further comprising hybridizing a first terminal oligonucleotide probe downstream of the first interposing oligonucleotide probe to the first polynucleotide, and hybridizing a second terminal oligonucleotide probe upstream of the second interposing oligonucleotide probe to the second polynucleotide;
wherein the first terminal oligonucleotide probe comprises from 5′ to 3′:
i. a hybridization sequence complementary to a sequence of said first polynucleotide; and
ii. a primer binding sequence; and
wherein the second terminal oligonucleotide probe comprises from 3′ to 5′:
i. a hybridization sequence complementary to a sequence of said second polynucleotide; and
ii. a primer binding sequence.

4. The method of claim 3, further comprising extending the 3′ end of the hybridization sequence of said second terminal oligonucleotide probe with one or more polymerases thereby forming an extension product.

5. The method of claim 3, further comprising ligating the 5′ end of the first terminal oligonucleotide probe to the 3′ end of the adjacent extension product.

6. The method of claim 1, wherein the bridge oligonucleotide comprises from 3′ to 5′:
i. a first hybridization sequence complementary to a 3′ terminal sequence of the first independent polynucleotide;
ii. a linker sequence; and
iii. a second hybridization sequence complementary to a 5′ terminal sequence of the second independent polynucleotide.

7. The method of claim 1, wherein the 3′end of the bridge oligonucleotide comprises about 5 to about 50 nucleotides complementary to the 3′ end of the first independent polynucleotide, and wherein the 5′end of the bridge oligonucleotide comprises about 5 to about 50 nucleotides complementary to the 5′ end of the second independent polynucleotide.

8. A method of amplifying a tagged complement of two independent single-stranded polynucleotides, said method comprising:
a. hybridizing a first overlap oligonucleotide to a first independent polynucleotide and a second overlap oligonucleotide to a second independent polynucleotide, and extending both the first and second overlap oligonucleotides with a polymerase, thereby forming an overlapped polynucleotide complex, wherein the overlapped polynucleotide complex comprises a complement of the first independent polynucleotide, the first overlap oligonucleotide, the second overlap oligonucleotide, and a complement of the second independent polynucleotide, wherein a 5′ sequence of the first overlap oligonucleotide is hybridized to a 5′ sequence of the second overlap oligonucleotide;
b. linking said overlapped polynucleotide complex, thereby generating a bridged polynucleotide comprising a sequence of the first independent polynucleotide linked to a sequence of the second independent polynucleotide or a complement thereof;
c. hybridizing to the bridged polynucleotide one or more interposing oligonucleotide barcodes, wherein each of the interposing oligonucleotide barcodes comprises from 5′ to 3′: i. a first hybridization sequence complementary to a first sequence of said bridged polynucleotide; ii. a loop region comprising a barcode; and iii. a second hybridization sequence complementary to a second sequence of said bridged polynucleotide;
d. extending the 3′ end of each second hybridization sequence of said interposing oligonucleotide barcodes with one or more polymerases thereby forming an extension product of each of said interposing oligonucleotide barcodes;
e. ligating the 3′ end of each of said extension products to the 5′ end of the adjacent extension products hybridized to the bridged polynucleotide thereby making an integrated strand tagged with a plurality of interposing oligonucleotide barcodes, wherein the integrated strand comprises sequences of the first and second independent polynucleotides or complements thereof; and
f. amplifying the integrated strand by an amplification reaction to produce a tagged complement of two independent single-stranded polynucleotides.

9. The method of claim 8, wherein each interposing oligonucleotide barcode comprises a first stem region comprising a sequence common to the plurality of interposing oligonucleotide barcodes and a second stem region comprising a sequence complementary to the first stem region, wherein the second stem region is capable of hybridizing to the first stem region under hybridization conditions.

10. The method of claim 8, wherein the first overlap oligonucleotide comprises from 5′ to 3′ a first hybridization sequence complementary to a 5′ sequence of the second overlap oligonucleotide, and a second hybridization sequence complementary to a 3′ sequence of the first independent polynucleotide, and wherein the second overlap oligonucleotide comprises from 5′ to 3′ a first hybridization sequence complementary to a 5′ sequence of the first overlap oligonucleotide and a second hybridization sequence complementary to a 3′ sequence of the second independent polynucleotide.

11. The method of claim 8, wherein prior to step (i), the method further comprises isolating a cell comprising a plurality of polynucleotides, wherein the plurality of polynucleotides comprises the first independent polynucleotide and the second independent polynucleotide.

12. The method of claim 8, wherein the barcode sequence of each interposing oligonucleotide barcode, alone or in combination with a sequence of one or both of (a) the bridged polynucleotide, or (b) one or more additional barcode sequences, distinguishes the bridged polynucleotide from bridged polynucleotides generated from other cells.

13.-15. (canceled)

16. The method of claim 8, wherein each of the interposing oligonucleotide barcodes comprise a phosphorylated 5′ end.

17. The method of claim 8, wherein the method comprises phosphorylating the 5′ ends of the interposing oligonucleotide barcodes prior to step c.

18. The method of claim 8, further comprising hybridizing to the bridged polynucleotide a terminal adapter, wherein the terminal adapter comprises a first hybridization sequence complementary to a first sequence of the bridged polynucleotide, a barcode sequence, and a primer binding sequence.

19.-21. (canceled)

22. A method of forming an integrated strand complement of a bridged polynucleotide comprising a plurality of oligonucleotide probes, wherein the bridged polynucleotide comprises a complement of two independent single-stranded polynucleotides, the method comprising:
a. hybridizing a bridge oligonucleotide to a first independent polynucleotide and to a second independent polynucleotide, thereby forming a bridged polynucleotide complex;
b. amplifying said bridged polynucleotide complex, thereby generating a bridged polynucleotide comprising a sequence of the first independent polynucleotide linked to a sequence of the second independent polynucleotide, or a complement thereof;
c. hybridizing one or more interposing oligonucleotide probes to the bridged polynucleotide, hybridizing a first terminal oligonucleotide probe downstream of the one or more interposing oligonucleotide probes, and hybridizing a second terminal oligonucleotide probe upstream of the one or more interposing oligonucleotide probes,
wherein each of the interposing oligonucleotide probes comprises from 5′ to 3′: i. a first hybridization sequence complementary to a first sequence of said bridged polynucleotide; ii. a loop region comprising a primer binding sequence; and iii. a second hybridization sequence complementary to a second sequence of said bridged polynucleotide;
wherein the first terminal oligonucleotide probe comprises from 5′ to 3′: i. a hybridization sequence complementary to a third sequence of said bridged polynucleotide; and ii. a primer binding sequence; and
wherein the second terminal oligonucleotide probe comprises from 3′ to 5′: i. a hybridization sequence complementary to a fourth sequence of said bridged polynucleotide; and ii. a primer binding sequence;
d. extending the 3′ end of each second hybridization sequence of said interposing oligonucleotide probes and the 3′ end of the hybridization sequence of said second terminal oligonucleotide probe with one or more polymerases thereby forming an extension product of each of said oligonucleotide probes;
e. ligating the 3′ end of each of said extension products to the 5′ end of the adjacent extension products, and ligating the 5′ end of the first terminal oligonucleotide probe to the 3′ end of the adjacent extension product, each hybridized to the same bridged polynucleotide thereby making an integrated strand comprising a complement of the bridged polynucleotide comprising a plurality of the oligonucleotide probes; and
f. amplifying the integrated strand by an amplification reaction to produce a complement of the integrated strand thereby forming an integrated strand complement of the bridged polynucleotide comprising oligonucleotide probes, wherein the complement of the integrated strand comprises a complement of the plurality of oligonucleotide probes.

23. The method of claim 22, wherein the bridge oligonucleotide comprises, from 3′ to 5′, a first hybridization sequence complementary to a 3′ sequence of the first independent polynucleotide, and a second hybridization sequence complementary to a 5′ sequence of the second independent polynucleotide.

24. The method of claim 22, wherein prior to step (a), the method further comprises isolating a cell comprising a plurality of polynucleotides, wherein said plurality of polynucleotides comprises the first independent polynucleotide and the second independent polynucleotide.

25. The method of claim 22, further comprising sequencing the amplified product of step (f).

26. The method of claim 25, wherein the sequencing further comprises (a) producing a plurality of sequencing reads; (b) grouping sequencing reads based on co-occurrence of interposing oligonucleotide probe sequences; and (c) within each group, aligning the sequencing reads that belong to the same strand of an original bridged polynucleotide based on the sequences of the interposing oligonucleotide probe sequences.

27.-50. (canceled)


SEQUENCE LISTING (8 sequences)

SEQ ID NO: 1 - skipped

SEQ ID NO: 2 - DNA, 15 bp, synthetic construct
  misc_feature 1..15: note = synthetic polynucleotide
  misc_feature 1..15
  source 1..15: mol_type = other DNA
cctattacgataaca

SEQ ID NO: 3 - DNA, 11 bp, synthetic construct
  misc_feature 1..11: note = synthetic polynucleotide
  misc_feature 1..11
  modified_base 7: mod_base = OTHER; note = locked nucleic acid (LNA)
  source 1..11: mol_type = other DNA
accacggtcac

SEQ ID NO: 4-6 - skipped (3 entries)

SEQ ID NO: 7 - DNA, 11 bp, synthetic construct
  misc_feature 1..11: note = synthetic polynucleotide
  misc_feature 1..11
  modified_base 4: mod_base = OTHER; note = locked nucleic acid (LNA)
  source 1..11: mol_type = other DNA
cgtctcctcag

SEQ ID NO: 8 - DNA, 10 bp, synthetic construct
  misc_feature 1..10: note = synthetic polynucleotide
  misc_feature 1..10
  source 1..10: mol_type = other DNA
agcctgcctg